(19) United States
(12) Patent Application Publication
(10) Pub. No.: US 2025/0361530 A1
(43) Pub. Date: Nov. 27, 2025

(54) GENOME EDITING SYSTEMS FOR MULTIPLEXING POINT MUTATION INTRODUCTION IN LIVING CELLS

(71) Applicant: The Regents of the University of California, Oakland, CA (US)

(72) Inventors: Alexis C. Komor, La Jolla, CA (US); Quinn Cowan, La Jolla, CA (US)

(21) Appl. No.: 18/865,618

(22) PCT Filed: May 10, 2023
(86) PCT No.: PCT/US2023/066812

§ 371 Date: Nov. 13, 2024

Related U.S. Application Data
(60) Provisional application No. 63/342,071, filed on May 14, 2022.

(51) Int. Cl. C12N 15/90; C12N 9/22; C12N 9/78; C12N 15/11

(52) U.S. Cl. CPC C12N 15/907; C12N 9/226; C12N 9/78; C12N 15/11; C12Y 305/04001; C12Y 305/04005; C12N 2310/20; C12N 2310/531

(57) ABSTRACT

The base editor systems (“MOBE”) that are derived from the CRISPR/Cas9 protein that enable to simultaneously introduce C⋅G to T⋅A and A⋅T to G⋅C point mutations at distinct genomic loci in living cells, with high efficiency and precision. In the MOBE disclosed herein, a piece of RNA (“the gRNA”) of the CRISPR/Cas9 protein is fused to the deaminase enzymes via a coat protein-aptamer interaction. A reporter plasmid comprising the MOBE system that allows for enrichment of cells with co-occurring orthogonal edits and increased editing efficiency.

### CROSS-REFERENCE TO RELATED APPLICATION

This application claims the benefit of U.S. Provisional Application No. 63/342,071, filed on May 14, 2022, the entire content of which is incorporated herein by reference.

### STATEMENT REGARDING FEDERALLY SPONSORED RESEARCH OR DEVELOPMENT

This invention was made with government support under 1R35GM138317 awarded by the National Institute of Health (NIH)/National Institute of General Medical Science (NIGMS). The government has certain rights in the invention.

### BACKGROUND

Advances in next-generation sequencing (NGS) have made the detection of human genetic variants increasingly routine. However, the ability to interpret the functional consequences of these variants has lagged far behind.¹ Specifically, while 241 million genetic variants have been reported in the Genome Aggregation Database, less than 1% have a clinical interpretation in ClinVar. Furthermore, 99% of identified variants are rare or population-specific, causing the prediction of their functional impact using genome-wide association studies (GWAS) or computational methods to be particularly challenging.² Additionally, the co-occurrence of variants further convolutes their interpretation using existing computational methods. In particular, different background genetic variations (such as those found in patient-derived cell lines) can lead to misclassification of risk-association on ClinVar or conflicting interpretations (in fact, 17% of variants submitted by more than one lab have conflicting interpretations). In these settings, GWAS fail to accurately determine whether individual variants are pathogenic or simply co-inherited as passenger mutations.

This presents a pressing unmet need in the field of genome editing to facilitate modeling of combinations of genetic variants within otherwise isogenic backgrounds. Functional investigation of specific variant combinations would enable researchers to deconvolute the clinical contributions of individual variants observed in polygenic disorders, those inherited in haplotype blocks, combinations of variants of uncertain significance (VUS), or variants in modifier genes within the same biological pathway. As single nucleotide variants (SNVs) account for 96% of observed human genetic variation, the development of programmable tools that can efficiently multiplex the installation of point mutations would be transformative for modelling and functionally characterizing genetic variants.

Traditional genome editing methods utilize programmable DNA double-strand breaks (DSBs) to precisely introduce SNVs. These tools, such as the wild-type CRISPR-Cas9 system, additionally rely on the homology-directed repair (HDR) pathway to incorporate desired modifications into the genome using an exogenously supplied donor DNA template. However, end-joining repair pathways, which are more ubiquitous and efficient than HDR, compete to process DSBs, resulting in the introduction of insertions and deletions (indels) of bases at the site of the DSB. DSB-reliant genome editing methods therefore suffer from high rates of unwanted gene alterations (indels) and low efficiencies of the desired modification, particularly when installing point mutations.⁴ When attempting to multiplex point mutation introduction with Cas9, the low precision of DSB-reliant technologies is exacerbated, as success rates decrease exponentially with the number of desired edits. Additionally, the incidences of translocations (when multiplexing at distinct chromosomes), large-scale deletions (when multiplexing within the same chromosome), chromosomal aberrations, and/or p53-mediated apoptosis increase when introducing multiple DSBs. Overall, the enhanced cytotoxicity and high rates of undesired editing outcomes make the use of DSB-reliant tools impractical for multiplexing._{5,6}

“Nontraditional” precision genome editing tools are those that avoid the introduction of DSBs, and instead use alternative DNA damage products as intermediates.⁷⁻⁹ Specifically, base editors (BEs) are comprised of a catalytically impaired Cas9 nickase (nCas9) covalently tethered to a single-stranded DNA (ssDNA) modifying enzyme (FIG. 1A—prior art). The Cas9 enzyme complexes with a guide RNA (gRNA) molecule, which directs the fusion protein to the target site (called the protospacer) via base-pairing rules. The protospacer must be directly next to a protospacer adjacent motif (PAM) for the Cas9:gRNA complex to bind. The Cas9:gRNA:DNA ternary complex is an R-loop, in which one DNA strand is base-paired with the gRNA, and the other is single-stranded and lacks a complement.¹⁰ This in turn exposes a small (˜5 nucleotide) “window” of accessible ssDNA (on the strand not bound by the gRNA) where the ssDNA modifying enzyme directly chemically modifies target nucleotides within this window (FIG. 1A—prior art). Two major classes of base editors have been developed that use cytosine and adenine deamination chemistries to catalyze the conversion of C⋅G base pairs to T⋅A (CBEs), and A⋅T base pairs to G⋅C (ABEs), respectively.^{11,12} These two types of transition point mutations account for 61% of observed SNVs, allowing for functional investigation of the majority of genetic variants using current BEs. BEs are uniquely situated to enable multiplexed genome editing as they have high on-target efficiencies (up to 90% simultaneous edits at 3 endogenous loci has been reported) and low byproduct formation.¹³ However, multiplexed base editing can currently only be performed in a straight-forward manner when using only a single BE (either ABE or CBE). Multiplexing CBEs and ABEs together currently faces several challenges.

BEs that employ the Streptococcus pyogenes (Sp) Cas9 homolog are the most widely-used BE variants due to their high efficiencies, narrow editing windows (which reduces bystander editing), and relatively flexible PAM requirements (there are engineered SpCas9s with NG and NR/Y PAMs; this allows for facile positioning of the target base in the center of the editing window).^{14,15} Attempts to multiplex Sp-derived CBEs and ABEs via nucleic acid-mediated delivery would result in “gRNA crosstalk,” in which all SpCas9 components would complex with all Sp-gRNAs, causing both C⋅G to T⋅A and A⋅T to G⋅C editing at all targeted genomic loci (FIGS. 1B-1C—prior art).

This crosstalk can be mitigated by pre-complexing each base editor protein with its appropriate gRNA in vitro, followed by direct ribonucleoprotein (RNP) delivery into cells. However, the difficulty in expressing and purifying base editor protein combined with a lack of commercial sources of base editor protein make this strategy inaccessible to most laboratories. It is currently possible to orthogonally multiplex ABEs and CBEs by using BEs that employ Cas9 orthologs that utilize distinct gRNA backbones, such as SpCas9 and Staphylococcus aureus (Sa) Cas9.¹⁶ Unfortunately, BEs derived from SaCas9 have more restrictive PAM requirements (NNNRRT for the most relaxed KKH variant) and much wider editing windows (resulting in bystander editing), which severely limits their utility for disease modelling and therapeutic SNV correction.¹⁷

### SUMMARY

A strategy in which the ssDNA modifying enzymes are directly recruited to their respective gRNAs (which encode the loci to be edited) would offer a modular system for multiplexed orthogonal base editing in which both BEs use the advantageous SpCas9 homolog and that is compatible with nucleic acid-based delivery methods (see e.g., as illustrated in FIG. 1D). Therefore, the present disclosure provides four multiplexed orthogonal base editor (MOBE) systems in which RNA aptamers are utilized to engineer, and which enable the simultaneous introduction of C⋅G to T⋅A SNVs and A⋅T to G⋅C SNVs at distinct protospacer with minimal crosstalk. These systems can be delivered via nucleic acid-mediated methods (plasmid or mRNA and synthetic gRNA) and utilize only a single Cas enzyme. In certain embodiments, the MOBE system of the present disclosure comprises a combination of aptamer-based Cytidine-BE system and an aptamer-based Adenine-BE system. In certain embodiments, each aptamer-based BE system comprises aptamer-gRNA constructs that are combined with corresponding coat protein-deaminase fusions.

In certain embodiments, the present disclosure provides a multiplexed orthogonal base editor (MOBE) system that comprises one or more an aptamer-based base editor (BE) system. The aptamer-based EB system comprises an aptamer-gRNA construct in which a DNA modifier recruited directly to its gRNA of a CRISPR/Cas9 protein via an aptamer-binding interaction. The aptamer-gRNA construct is then combined with a corresponding coat protein-deaminase fusion of the CRISPR/Cas9 protein.

In certain embodiments, the present disclosure provides four MOBE systems, namely, MOBE1, MOBE2, MOBE3, and MOBE4, each of which is a Sp-nCas9 variant and comprises a combination of a Cytidine-BE system and an Adenine-BE system (see FIG. 5A). The construct of each MOBE is further illustrated in FIGS. 25A-25D with the sequence information of each MOBE being presented in the Table in Detailed Description below.

The MOBE systems of the present disclosure are on average 25-fold more orthogonal (as assessed by comparing on-target editing to crosstalk editing when multiplexing) than when multiplexing with current Cytidine-BE (CBE) and Adenine-BE (ABE) systems. Additionally, without any selection or enrichment strategies, the MOBE systems of the present disclosure achieve co-occurring orthogonal editing rates of up to 5.5% with crosstalk rates of only 1.3% when multiplexing with two protospacers. A fluorescence-based reporter plasmid was additionally developed that facilitates the enrichment of cells with high MOBE activity. When using the MOBE systems with this enrichment strategy, up to 25.3% co-occurring orthogonal edits were achieved with crosstalk rates of only 1.1%.

The way current base editors work is that the deaminase enzymes (which do the nucleobase chemistry) are directly fused to the Cas9 enzyme. A piece of RNA (called the gRNA) is then programmed to direct the Cas9 enzyme to particular genomic loci. If an ABE and a CBE are multiplexed and added to the cell with multiple gRNAs that encode for the various genomic loci where editing is desired, both the CBE and the ABE complex with all the gRNAs, resulting in both C⋅G to T⋅A and A⋅T to G⋅C editing at all the loci. However, in the MOBE systems of the present disclosure, each different gRNA was “tagged” with an aptamer (a particular RNA sequence—a separate aptamer for the CBE system and the ABE system). The aptamer then essentially marks each gRNA as a CBE or ABE gRNA. The cytidine deaminase (for the CBE), and the adenosine deaminase (for the ABE) is then tethered to the aptamer's “coat protein” partner (which binds with nM affinity to the partner aptamer). The individual aptamer-coat protein systems are orthogonal to each other, so the ABE deaminase does not complex with the CBE aptamer. These are combined with a universal Cas9 protein, which then takes each gRNA-aptamer-coat protein-deaminase complex to its proper genomic loci, and only C⋅G to T⋅A or A⋅T to G⋅C editing occurs at each site.

The present disclosure provides for the first-time base editors that create orthogonal point mutations at distinct genomic sites in human cells. The base editor MOBE systems provided by the present disclosure can be used for therapeutic correction of polygenic disorders, modeling of polygenic disorders, and other gene editing for treatment.

Other systems, methods, features, and advantages of the present disclosure can be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWING

Many aspects of the present disclosure can be better understood with reference to the following drawings (also “Figures” or “FIGs”). The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D. Schematic overview of multiplexed orthogonal base editing. FIG. 1A (prior art). Overview of base editing, with the cytosine base editor (CBE) shown as an example. Base editors are comprised of a ssDNA modifying enzyme (dark grey, a cytosine deaminase in CBEs) tethered to a catalytically impaired Cas9 (nCas9, light grey). Binding to genomic DNA is facilitated by base-pairing between the spacer region of the gRNA and a complementary protospacer sequence in the DNA. The protospacer must also be next to a PAM motif (the PAM sequence is NG for all experiments described herein). DNA binding by the Cas9:gRNA complex forms an R-loop, which exposes a ˜5 nucleotide window to the ssDNA cytosine deaminase. Any cytosines within this window are deaminated to uracils, and the opposite DNA strand is nicked by nCas9. The U⋅G intermediate is subsequently processed by the cell to produce an overall C⋅G to T⋅A base pair conversion. The adenine base editor (ABE) works analogously but facilitates A⋅T to G⋅C base pair conversions via inosine-containing intermediates. FIG. 1B (prior art). When multiplex a CBE and an ABE, gRNA crosstalk occurred. Delivery of the BEs and gRNAs via nucleic acid-based methods (plasmid-encoded or mRNA and synthetic gRNA) results in both the CBE and ABE complexing with both gRNAs in situ. This subsequently results in both C⋅G to T⋅A and A⋅T to G⋅C editing at both protospacers. FIG. 1C (prior art). Schematic representation of possible genotypes when multiplex C⋅G to T⋅A and A⋅T to G⋅C editing at two neighboring protospacers. FIG. 1D. Schematic representation of an aptamer-based system for multiplexed orthogonal base editing. A single nCas9 variant is used for both editors, and the cytidine and adenosine deaminase enzymes are recruited directly to their respective gRNAs via orthogonal aptamer-coat protein interactions. As a result, only C⋅G to T⋅A editing occurred at the desired CBE target protospacer, and only A⋅T to G⋅C editing occurred at the desired ABE target protospacer.

FIGS. 2A-2E. Engineering of an ABE aptamer system. FIGS. 2A, 2B, and 2D. Editing efficiencies at the HIRA and RNF2 loci of ABE aptamer systems employing the TadA8e (FIGS. 2A and 2D) or TadA8.20 (FIGS. 2B and 2D) deaminase and PP7, boxB, or com aptamer-coat protein systems. HEK293T cells were transfected with plasmids encoding nCas9-NG, TadA8-CP fusion, and gRNA-aptamer (for aptamer-treated cells), or plasmids encoding the parental ABE8e-NG or ABE8.20-NG editors and unmodified gRNA (for ABE8e and ABE8.20-treated cells). Cells were then lysed after 72 hours, genomic loci of interest were amplified, and analyzed by NGS. Plotted are the percent of total DNA sequencing reads with A⋅T edited to G⋅C at the target A's indicated. Bar graphs (FIGS. 2A and 2B) and dot plots (FIG. 2D) represent the average, and error bars represent the standard deviation for n=3 biological replicates (each replicate is marked individually in FIGS. 2A and 2B). Negative control (NC) samples are also shown, in which cells were not transfected with any plasmid. FIG. 2C. Schematics of the aptamer-embedded gRNA and deaminase-coat protein fusions tested in FIGS. 2A, 2B, and 2D. FIG. 2E. Schematics of the two most efficient aptamer-embedded gRNA and deaminase-coat protein fusions selected as final ABE aptamer systems.

FIGS. 3A-3E. Engineering of a CBE aptamer system. FIGS. 3A and 3B. Editing efficiencies at the HEK3 and RNF2 loci of CBE aptamer systems employing the evoAPOBEC1 deaminase and MS2 aptamer-coat protein system. Cells were treated as previously described in FIGS. 2A-2E. Plotted are the percent of total DNA sequencing reads with C⋅G edited to T⋅A at the target C's indicated. Bar graphs (FIG. 3A) and dot plots (FIG. 3B) represent the average, and error bars represent the standard deviation for n=3, 4, or 5 biological replicates (each replicate is marked individually in FIG. 3A). Negative control (NC) samples are also shown, in which cells were not transfected with any plasmid. In FIG. 3B, the top seven constructs are indicated with the grey box. FIG. 3C. Schematics of the aptamer-embedded gRNA and deaminase-coat protein fusions tested in FIGS. 3A and 3B. FIG. 3D. Editing efficiencies at the HEK3, EMX1, RNF2, HEK2, and HIRA loci of the top seven CBE aptamer systems employing the evoAOBEC1 deaminase and the MS2 aptamer-coat protein system from FIGS. 3A and 3B. Shown are the percent of total DNA sequencing reads with C⋅G edited to T⋅A at the target C's indicated. Values represent the average for n=3, 4, or 5 biological replicates. The two constructs that consistently displayed the highest editing efficiencies across all five sites are indicated with arrows. FIG. 3E. Schematics of the two most efficient aptamer-embedded gRNA and deaminase-coat protein fusions that were selected as final CBE aptamer systems.

FIGS. 4A-4D. Characterization of optimized CBE and ABE aptamer systems. FIGS. 4A and 4B. Editing efficiencies by all four optimized aptamer systems and their respective parental editors at 76 Cs and 116 As, measured from 15 different protospacers. Target Cs and As are organized by their position within the protospacer (x-axis). Cells were treated as previously described in FIGS. 2A-2E. Plotted are the percent of total DNA sequencing reads with C⋅G edited to T⋅A (FIG. 4A) or A⋅T edited to G⋅C (FIG. 4B). Bar graphs and error bars represent the average and standard deviation across all measured Cs or As at that position (n=3 biological replicates for each target C or A, and each replicate is marked individually). FIGS. 4C and 4D. Editing efficiencies by all four optimized aptamer systems normalized to their respective parental editor. FIG. 4C. For each CBE aptamer system, the highest edited C within each of the 15 protospacers was normalized to the parental editor by dividing the percent of total DNA sequencing reads with the target C⋅G edited to T⋅A for the aptamer system by that of the parental editor. FIG. 4D. For each ABE aptamer system, the highest edited A within each of the 15 protospacers was normalized to the parental editor by dividing the percent of total DNA sequencing reads with the target A⋅T edited to G⋅C for the aptamer system by that of the parental editor. Values on the whisker plots represent the lowest observation, lower quartile, median, upper quartile and the highest observation across the 15 different sites.

FIGS. 5A-5D. On-target and crosstalk editing efficiencies of MOBEs and parental BEs when multiplexing CBE and ABE. FIG. 5A. The four aptamer CBE-ABE combinations that make up each of the four multiplexed orthogonal base editor (MOBE) systems are specified. The construct schematics of the aptamer CBE and ABE systems are listed in FIGS. 3A-3E and 2A-2E, respectively. FIGS. 5B and 5C. Editing efficiencies by the MOBE1-4 systems and the evoBE4-NG/ABE8e-NG and evoBE4-NG/ABE8.20-NG combinations at the HIRA.0/HEK3.0 (FIG. 5B) and HEK3.2/HEK3.0 (FIG. 5C) loci. HEK293T cells were transfected with plasmids encoding nCas9-NG-P2A-mCherry, tandem CP-deaminase fusions, and both gRNA-aptamers (for the MOBE systems), or plasmids encoding the parental evoBE4-NG, ABE8e-NG or ABE8.20-NG, and both unmodified gRNAs (for the evoBE4-NG/ABE8e-NG and evoBE4-NG/ABE8.20-NG combinations). Cells were then lysed after 72 hours, genomic loci of interest were amplified, and analyzed by NGS. Plotted are the percent of total DNA sequencing reads with A⋅T edited to G⋅C at the ABE target (y-axis, solid circles), C⋅G edited to T⋅A at the ABE target (y-axis, circles with X inset), C⋅G edited to T⋅A at the CBE target (x-axis, solid circles), and A⋅T edited to G⋅C at the CBE target (x-axis, circles with X inset). Dot plots and error bars represent the average and standard deviation for n=3 biological replicates. Negative control (NC) samples are also shown, in which cells were not transfected with any plasmid. FIG. 5D. Orthogonality scores for the MOBE1-4 systems and the evoBE4-NG/ABE8e-NG and evoBE4-NG/ABE8.20-NG combinations at the two protospacer combinations from (FIG. 5B) and (FIG. 5C). Plotted are the “CBE orthogonality scores”, which were defined for a given protospacer combination as the log 2 of the percent of total DNA sequencing reads with C⋅G edited to T⋅A at the CBE target divided by the percent of total DNA sequencing reads with C⋅G edited to T⋅A at the ABE target. The “ABE orthogonality scores” are the A⋅T to G⋅C editing efficiency equivalent. Dot plots and error bars represent the average and standard deviation for n=3 biological replicates.

FIGS. 6A-6D. On-target and crosstalk editing efficiencies of MOBEs when performing multiplexed editing followed by FACS enrichment for episomal edits. (A) Schematic diagram of the 2×-dead-GFP reporter. GFP fluorescence is only observed following successful multiplexed, orthogonal base editing within the GFP gene. GFP+ cells can then be sorted to enrich for cells with highly active base editors, which enhances editing at endogenous genomic loci. FIGS. 6B and 6C. Editing efficiencies by the MOBE1-4 systems at the HIRA.0/HEK3.0 (FIG. 6B) and HEK3.2/HEK3.0 (FIG. 6C) loci. HEK293T cells were transfected with plasmids encoding nCas9-NG-P2A-mCherry, tandem CP-deaminase fusions, both gRNA-aptamers targeting endogenous loci, and the 2×-dead-GFP reporter. After 96 hours, populations of both “unenriched” cells and GFP+/mCherry+“enriched” cells were collected by FACS. Genomic loci of interest were then amplified and analyzed by NGS. Plotted are the percent of total DNA sequencing reads with A⋅T edited to G⋅C at the ABE target (y-axis, solid circles), C⋅G edited to T⋅A at the ABE target (y-axis, circles with X inset), C⋅G edited to T⋅A at the CBE target (x-axis, solid circles), and A⋅T edited to G⋅C at the CBE target (x-axis, circles with X inset). Dot plots and error bars represent the average and standard deviation for n=3 biological replicates. Negative control (NC) samples are also shown, in which cells were not transfected with any plasmid. FIG. 6D. Plotted are the percent of total DNA sequencing reads with A⋅T edited to G⋅C at the ABE target and C⋅G edited to T⋅A at the CBE target, with no crosstalk edits, when all multiplexing systems are targeted to two protospacers within the HEK3 locus. The parental systems and the “bulk” samples were treated as previously described in FIGS. 5A-5D. Bar graphs and error bars represent the average and sem for n=3 biological replicates (each replicate is marked individually).

FIGS. 7A-7C. Initial testing of ABE aptamer systems with the wtTadA-TadA7.10 deaminase. FIG. 7A. Editing efficiencies at the HEK2, HIRA, and PSMB2 loci of ABE aptamer systems employing the wtTadA-TadA7.10 deaminase and the PP7 aptamer-coat protein system (top), with schematics of the aptamer-imbedded gRNAs and deaminase-coat protein fusions shown (bottom). FIG. 7B. Editing efficiencies at the HIRA and RNF2 loci of ABE aptamer systems employing the wtTadA-TadA7.10 deaminase and PP7, boxB, or com aptamer-coat protein systems (top), with schematics of the aptamer-imbedded gRNAs and deaminase-coat protein fusions shown (bottom). HEK293T cells were transfected with plasmids encoding nCas9-NG, wtTad-TadA7.10-CP fusion, and gRNA-aptamer (for aptamer-treated cells), or plasmids encoding the parental ABE7.10max-NG editor and unmodified gRNA (for ABE7.10max-NG-treated cells). Cells were then lysed after 72 hours, genomic loci of interest were amplified, and analyzed by next generation sequencing (NGS). Plotted are the percent of total DNA sequencing reads with A⋅T edited to G⋅C at the target A's indicated. Bar graphs and error bars represent the average and standard error of the mean (sem) for n=2 or 3 biological replicates (each replicate is marked individually). Negative control (NC) samples are also shown, in which cells were not transfected with any plasmid. FIG. 7C. Crystal structure (PDB ID 5Y36) of the Cas:gRNA:DNA R-loop complex, with the target single-stranded DNA indicated, and the various locations of the gRNA that protrude from the Cas9 protein (the tetraloop, stem-loop 2, stem-loop 3, and 3′end) indicated.

FIGS. 8A-8D. Optimization of ABE aptamer systems employing TadA8 deaminases. FIG. 8A. Editing efficiencies at the HIRA and RNF2 loci of ABE aptamer systems employing the TadA8e and TadA8.20 deaminases normalized to the parental ABE8e-NG or ABE8.20-NG constructs. Schematics of the aptamer-embedded gRNAs and deaminase-coat protein fusions are shown in FIG. 2C. Cells were treated as previously described in FIGS. 7A-7C. Plotted are the percent of total DNA sequencing reads with A⋅T edited to G⋅C at the target A's indicated for the aptamer-treated cells divided by that of the cells treated with the respective parental ABE8 construct. Bar graphs and error bars represent the average and propagation of uncertainty for n=3 biological replicates. FIGS. 8B and 8C. Editing efficiencies at the HIRA and RNF2loci of ABE aptamer systems with the com aptamer embedded at the 3′ end and TadA8e- or TadA8.20-Com fusion proteins. Cells were treated as previously described in FIGS. 7A-7C. Plotted are the percent of total DNA sequencing reads with A⋅T edited to G⋅C at the target A's indicated. Bar graphs (FIG. 8B) and dot plots (FIG. 8C) represent the average, and error bars represent the sem for n=3 or 4 biological replicates (each replicate is marked individually in FIG. 8C). Negative control (NC) samples are also shown, in which cells were not transfected with any plasmid. FIG. 8D. Schematics of the aptamer-embedded gRNA and deaminase-coat protein fusions tested in FIGS. 8B and 8C.

FIGS. 9A-9D. Initial testing of CBE aptamer systems with the ancAPOBEC, evoAOBEC1, and RrA3F deaminases. FIGS. 9A, 9B, and 9C. Editing efficiencies at the HEK3 (FIG. 9A), HIRA (FIG. 9B), and RNF2 (FIG. 9C) loci of CBE aptamer systems employing the ancAPOBEC, evoAOBEC1, or RrA3F deaminases and the MS2 aptamer-coat protein system. Cells were treated as previously described in FIGS. 7A-7C. Plotted are the percent of total DNA sequencing reads with C⋅G edited to T⋅A at the target C's indicated. Bar graphs and error bars represent the average and sem for n=3 or 5 biological replicates (each replicate is marked individually). Negative control (NC) samples are also shown, in which cells were not transfected with any plasmid. FIG. 9D. Schematics of the aptamer-embedded gRNAs and deaminase-coat protein fusions tested in FIGS. 9A, 9B, and 9C.

FIGS. 10A-10C. Architecture optimization of CBE aptamer systems employing the evoAPOBECi deaminase. FIG. 10A. The same graph from FIG. 3B, with all constructs labelled. FIGS. 10B and 10C. Editing efficiencies at the HEK2, EMX1, and HIRA loci of the top seven CBE aptamer systems employing the evoAOBEC1 deaminase and the MS2 aptamer-coat protein system. Schematics of the aptamer-embedded gRNA and deaminase-coat protein fusions are shown in FIG. 3C. Cells were treated as previously described in FIGS. 7A-7D. Plotted are the percent of total DNA sequencing reads with C⋅G edited to T⋅A at the target C's indicated. Bar graphs (FIG. 10B) and dot plots (FIG. 10C) represent the average, and error bars represent the sem for n=3 or 4 biological replicates (each replicate is marked individually in FIG. 10B). Negative control (NC) samples are also shown, in which cells were not transfected with any plasmid.

FIGS. 11A-11B. Characterization of optimized CBE and ABE aptamer systems. FIGS. 11A and 11B. Editing efficiencies, organized by position within the protospacer, of 38 Cs and 33 As by all four aptamer systems normalized to the respective parental editor. Cells were treated as previously described in FIGS. 7A-7C. FIG. 11A. Plotted are the average percent of total DNA sequencing reads with C⋅G edited to T⋅A at each target C by each CBE aptamer system, divided by that of the parental editor. Dot plots represent the average for n=3 biological replicates. FIG. 11B. Plotted are the average percent of total DNA sequencing reads with A⋅T edited to G⋅C at each target A by each ABE aptamer system, divided by that of the parental editor. Dot plots represent the average for n=3 biological replicates. Only target As and Cs that displayed average editing efficiencies >1% by the parental editor of are shown.

FIGS. 12A-12G. On-target and crosstalk editing efficiencies of MOBEs and parental BEs when targeted to two protospacers at distinct chromosomes. FIGS. 12A-12F. Editing efficiencies by the MOBE1-4 systems and the evoBE4-NG/ABE8e-NG and evoBE4-NG/ABE8.20-NG combinations at the HIRA.0/HEK3.0 (FIGS. 12A and 12D), HEK2.0/RNF2.0 (FIGS. 12B and 12E), and HIRA.3/RNF2.0 (FIGS. 12C and 12F) loci. HEK293T cells were transfected with plasmids encoding nCas9-NG-P2A-mCherry, tandem CP-deaminase fusions, and both gRNA-aptamers (for the MOBE systems), or plasmids encoding the parental evoBE4-NG, ABE8e-NG or ABE8.20-NG, and both unmodified gRNAs (for the evoBE4-NG/ABE8e-NG and evoBE4-NG/ABE8.20-NG combinations). Cells were then lysed after 72 hours, genomic loci of interest were amplified, and analyzed by NGS. FIGS. 12A-12C. Plotted are the percent of total DNA sequencing reads with A⋅T edited to G⋅C at the ABE target (y-axis, solid circles), C⋅G edited to T⋅A at the ABE target (y-axis, circles with X inset), C⋅G edited to T⋅A at the CBE target (x-axis, solid circles), and A⋅T edited to G⋅C at the CBE target (x-axis, circles with X inset). Dot plots and error bars represent the average and sem for n=3 biological replicates. FIGS. 12D-12F. Plotted are the percent of total DNA sequencing reads with A⋅T edited to G⋅C at the ABE target and C⋅G edited to T⋅A at the CBE target (on-target efficiencies only). Bar graphs and error bars represent the average and sem for n=3 biological replicates (each replicate is marked individually). Negative control (NC) samples are also shown, in which cells were not transfected with any plasmid. FIG. 12G. Orthogonality scores for the MOBE1-4 systems and the evoBE4-NG/ABE8e-NG and evoBE4-NG/ABE8.20-NG combinations at the three protospacer combinations from (FIG. 12A) through (FIG. 12C). Plotted are the “CBE orthogonality scores”, which were defined for a given protospacer combination as the log 2 of the percent of total DNA sequencing reads with C⋅G edited to T⋅A at the CBE target divided by the percent of total DNA sequencing reads with C⋅G edited to T⋅A at the ABE target. The “ABE orthogonality scores” are the A⋅T to G⋅C editing efficiency equivalent. Dot plots and error bars represent the average and standard deviation for n=3 biological replicates.

FIGS. 13A-13G. On-target and crosstalk editing efficiencies of MOBEs and parental BEs when targeted to two protospacers within the same locus. FIGS. 13A-13F. Editing efficiencies by the MOBE1-4 systems and the evoBE4-NG/ABE8e-NG and evoBE4-NG/ABE8.20-NG combinations at the HEK3 (FIGS. 13A and 13D), EMX1 (FIGS. 13B and 13E), and RNF2 (FIGS. 13C and 13F) loci. Cells were treated as previously described in FIGS. 12A-12G. FIGS. 13A-13C. Plotted are the percent of total DNA sequencing reads with A⋅T edited to G⋅C at the ABE target (y-axis, solid circles), C⋅G edited to T⋅A at the ABE target (y-axis, circles with X inset), C⋅G edited to T⋅A at the CBE target (x-axis, solid circles), and A⋅T edited to G⋅C at the CBE target (x-axis, circles with X inset). Dot plots and error bars represent the average and sem for n=3 biological replicates. FIGS. 13D-13F. Plotted are the percent of total DNA sequencing reads with A⋅T edited to G⋅C at the ABE target and C⋅G edited to T⋅A at the CBE target (on-target efficiencies only). Bar graphs and error bars represent the average and sem for n=3 biological replicates (each replicate is marked individually). Negative control (NC) samples are also shown, in which cells were not transfected with any plasmid. FIG. 13G. Orthogonality scores for the MOBE1-4 systems and the evoBE4-NG/ABE8e-NG and evoBE4-NG/ABE8.20-NG combinations at the three protospacer combinations from (FIG. 13A) through (FIG. 13C). Plotted are the “CBE orthogonality scores”, which were defined for a given protospacer combination as the log 2 of the percent of total DNA sequencing reads with C⋅G edited to T⋅A at the CBE target divided by the percent of total DNA sequencing reads with C⋅G edited to T⋅A at the ABE target. The “ABE orthogonality scores” are the A⋅T to G⋅C editing efficiency equivalent. Dot plots and error bars represent the average and standard deviation for n=3 biological replicates.

FIGS. 14A-14C. Quantification of co-occurring orthogonal edits. FIGS. 14A and 14B. Cells were treated as previously described in FIGS. 12A-12G (parental systems and “bulk” samples), or HEK293T cells were transfected with plasmids encoding nCas9-NG-P2A-mCherry, tandem CP-deaminase fusions, both gRNA-aptamers targeting endogenous loci, and the 2×-dead-GFP reporter. After 96 hours, populations of GFP+/mCherry+“enriched” cells were collected by fluorescence activated cell sorting (FACS). Genomic loci of interest were then amplified and analyzed by NGS (“enriched” samples). Plotted are the percent of total DNA sequencing reads with A⋅T edited to G⋅C at the ABE target and C⋅G edited to T⋅A at the CBE target, with no crosstalk edits when all multiplexing systems are targeted to two protospacers within the HEK3 (FIG. 14A), EMX1 (FIG. 14B), or RNF2 (FIG. 14C) locus. Bar graphs and error bars represent the average and sem for n=3 biological replicates (each replicate is marked individually).

FIG. 15. Genotype analysis of multiplexed base editing at the HEK3 locus. Cells were treated as previously described in FIGS. 14A-14C. Plotted are the percent of total DNA sequencing reads with the genotypes illustrated on the left when all indicated multiplexing systems are targeted to two protospacers within the HEK3 locus. Numerical values represent the average for n=3 biological replicates.

FIG. 16. Genotype analysis of multiplexed base editing at the EMX1 locus. Cells were treated as previously described in FIGS. 14A-14C. Plotted are the percent of total DNA sequencing reads with the genotypes illustrated on the left when all indicated multiplexing systems are targeted to two protospacers within the EMX1 locus. Numerical values represent the average for n=3 biological replicates.

FIG. 17. Genotype analysis of multiplexed base editing at the RNF2 locus. Cells were treated as previously described in FIGS. 14A-14C. Plotted are the percent of total DNA sequencing reads with the genotypes illustrated on the left when all indicated multiplexing systems are targeted to two protospacers within the RNF2locus. Numerical values represent the average for n=3 biological replicates.

FIGS. 18A-18F. On-target and crosstalk editing efficiencies of MOBEs when targeted to two protospacers at distinct chromosomes followed by FACS enrichment for episomal edits. FIGS. 18A-18F. Editing efficiencies by the MOBE1-4 systems at the HIRA.0/HEK3.0 (FIGS. 18A and 18D), HEK2.0/RNF2.0 (FIGS. 18B and 18E), and HIRA.3/RNF2.0 (FIGS. 18C and 18F) loci. HEK293T cells were transfected with plasmids encoding nCas9-NG-P2A-mCherry, tandem CP-deaminase fusions, both gRNA-aptamers targeting endogenous loci, and the 2×-dead-GFP reporter. After 96 hours, populations of both “unenriched” cells and GFP+/mCherry+“enriched” cells were collected by fluorescence activated cell sorting (FACS). Genomic loci of interest were then amplified and analyzed by NGS. FIGS. 18A-18C. Plotted are the percent of total DNA sequencing reads with A⋅T edited to G⋅C at the ABE target (y-axis, solid circles), C⋅G edited to T⋅A at the ABE target (y-axis, circles with X inset), C⋅G edited to T⋅A at the CBE target (x-axis, solid circles), and A⋅T edited to G⋅C at the CBE target (x-axis, circles with X inset). Dot plots and error bars represent the average and sem for n=3 biological replicates. FIGS. 18D-18F. Plotted are the percent of total DNA sequencing reads with A⋅T edited to G⋅C at the ABE target and C⋅G edited to T⋅A at the CBE target (on-target efficiencies only). Bar graphs and error bars represent the average and sem for n=3 biological replicates (each replicate is marked individually). Negative control (NC) samples are also shown, in which cells were not transfected with any plasmid.

FIGS. 19A-19F. On-target and crosstalk editing efficiencies of MOBEs when targeted to two protospacers within the same locus followed by FACS enrichment for episomal edits. FIGS. 19A-19F. Editing efficiencies by the MOBE1-4 systems at the HEK3 (FIGS. 19A and 19D), EMX1 (FIGS. 19B and 19E), and RNF2 (FIGS. 19C and 19F) loci. Cells were treated as previously described in FIGS. 18A-18F. FIGS. 19A-19C.

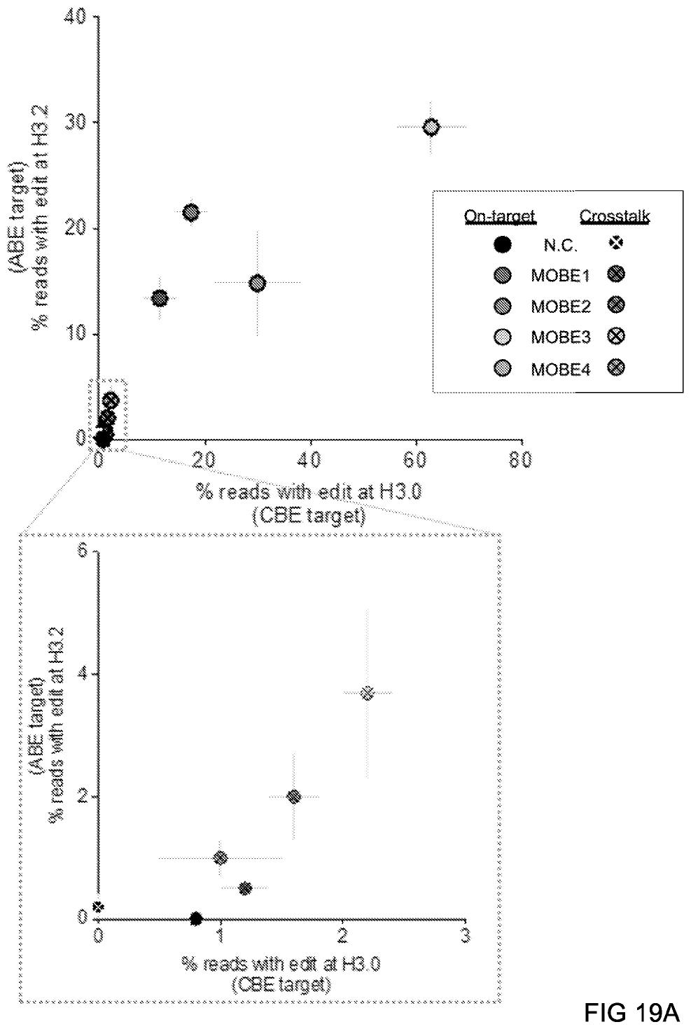

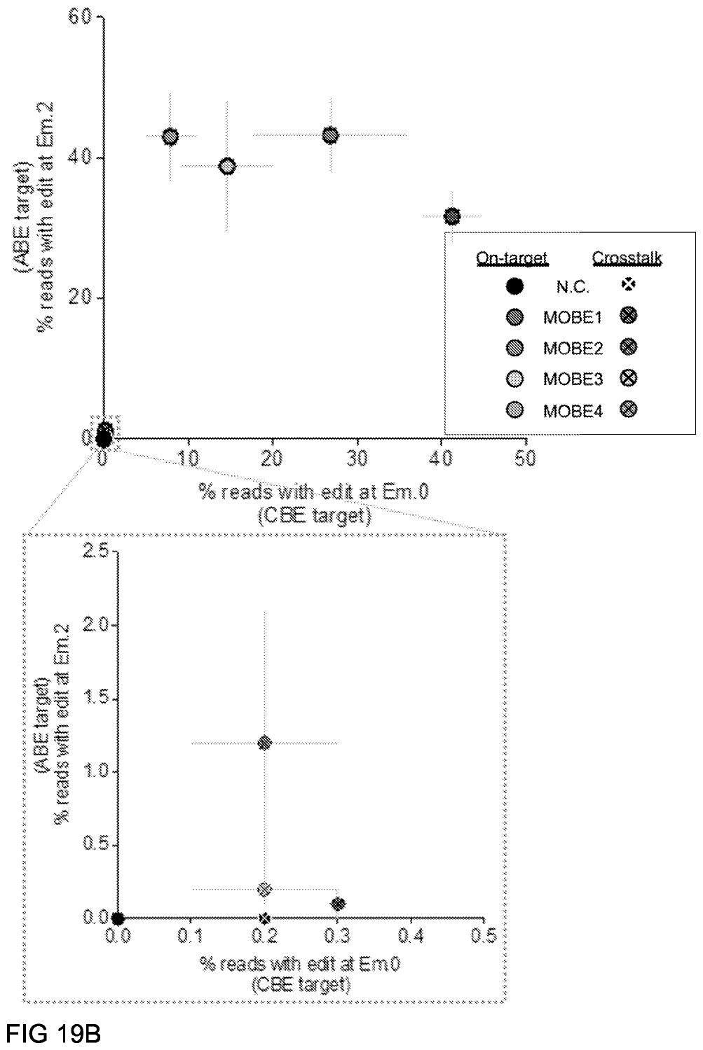

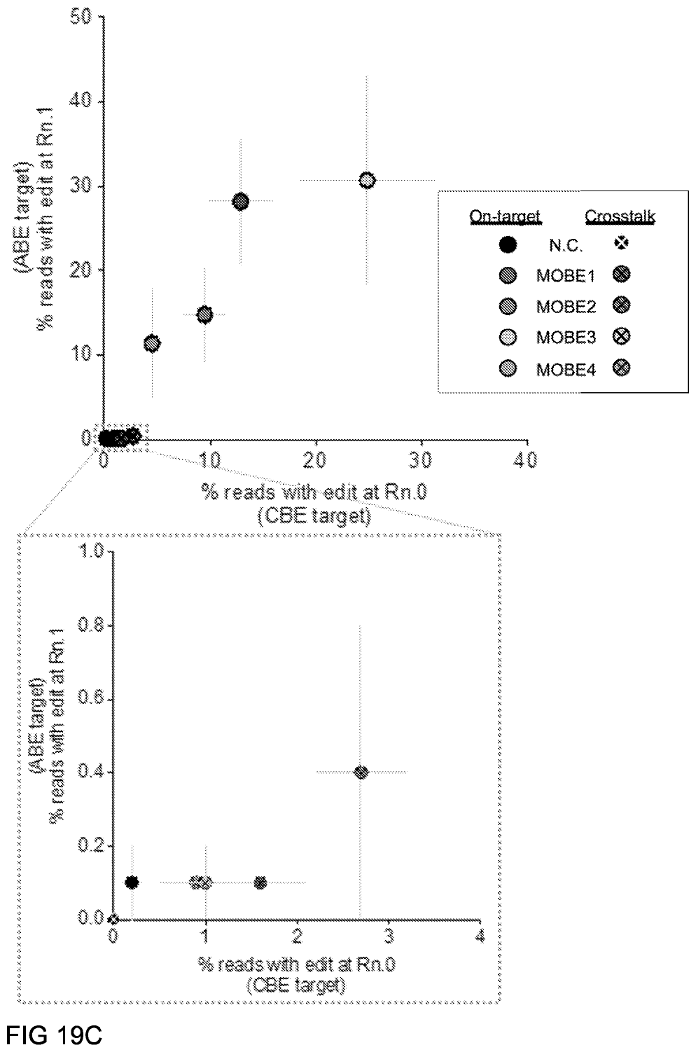

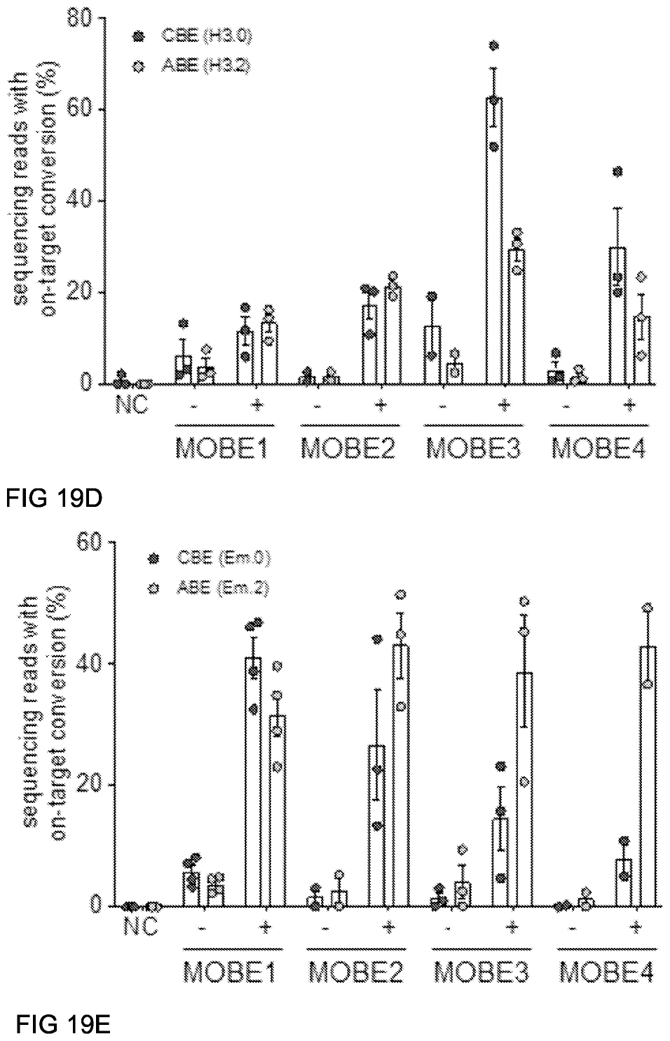

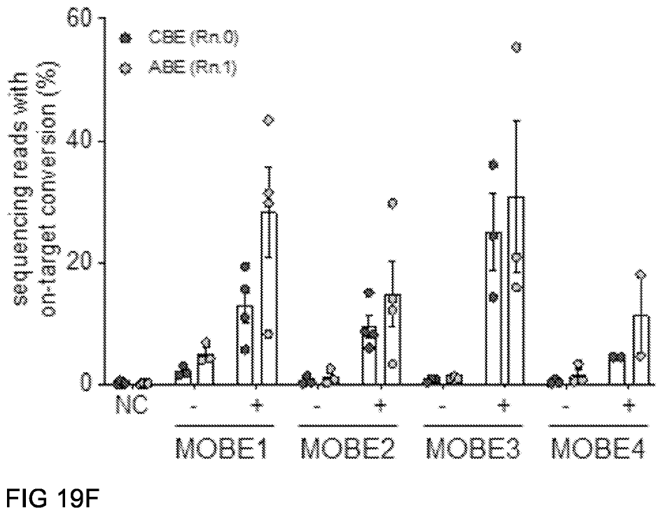

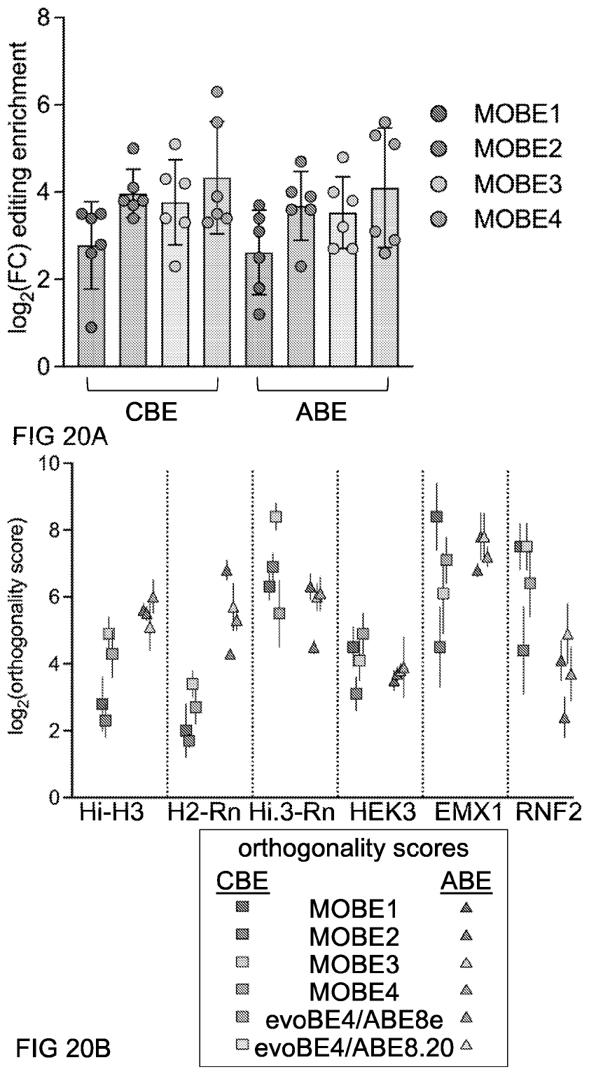

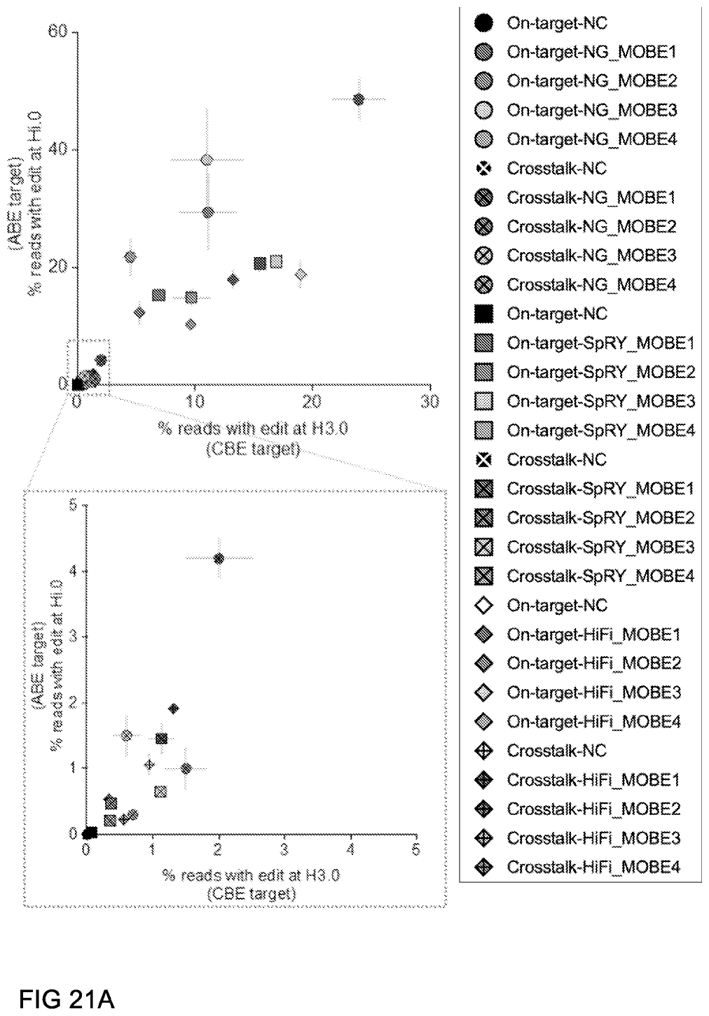

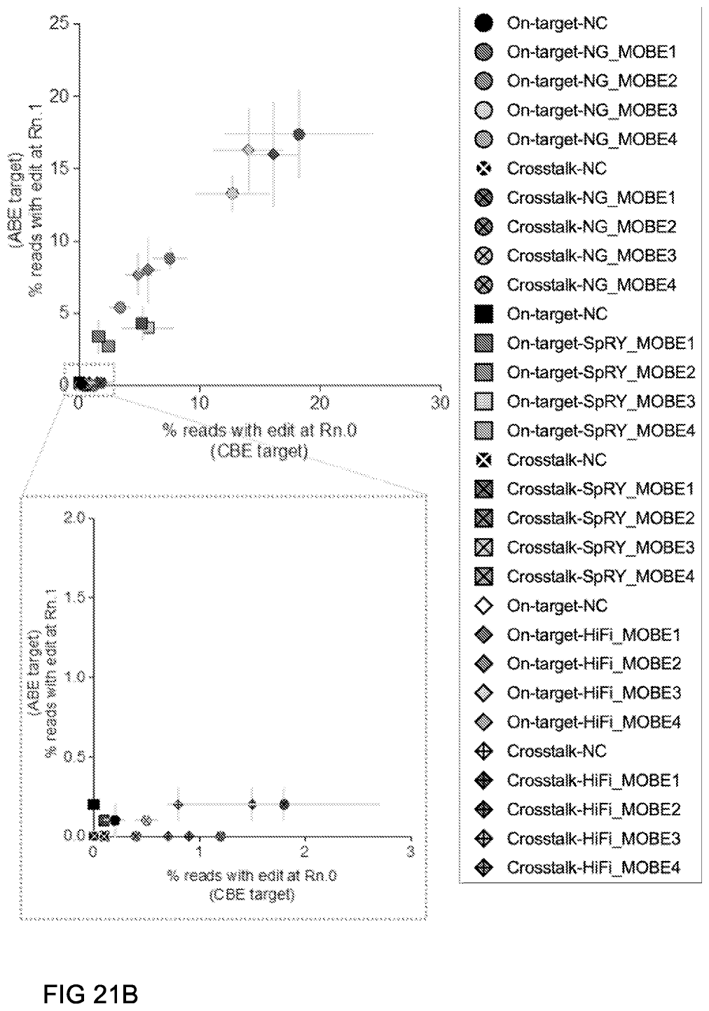

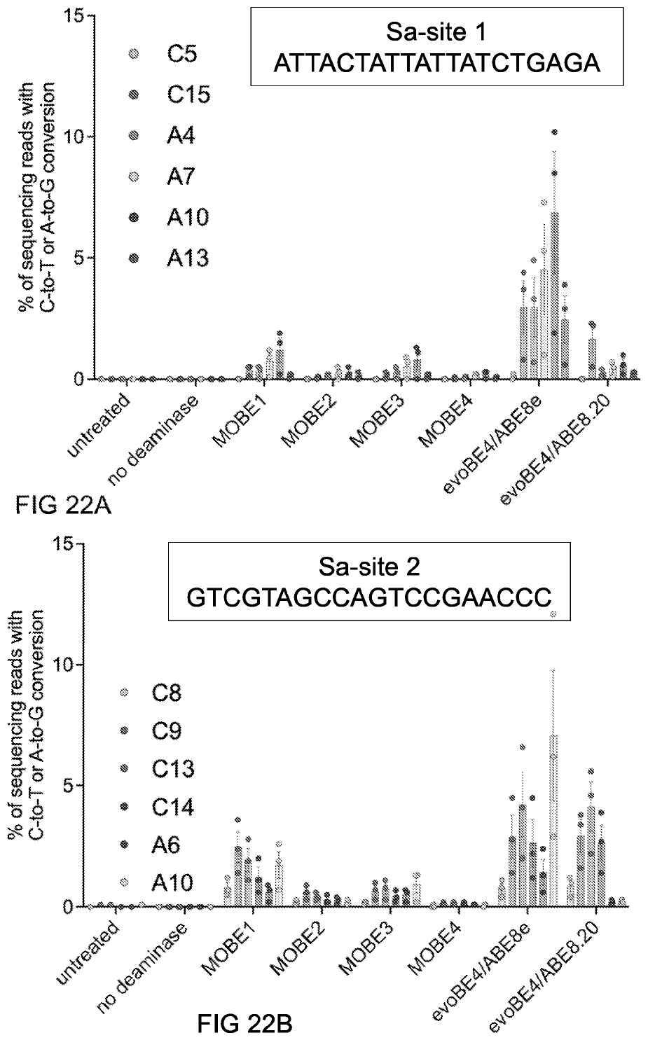

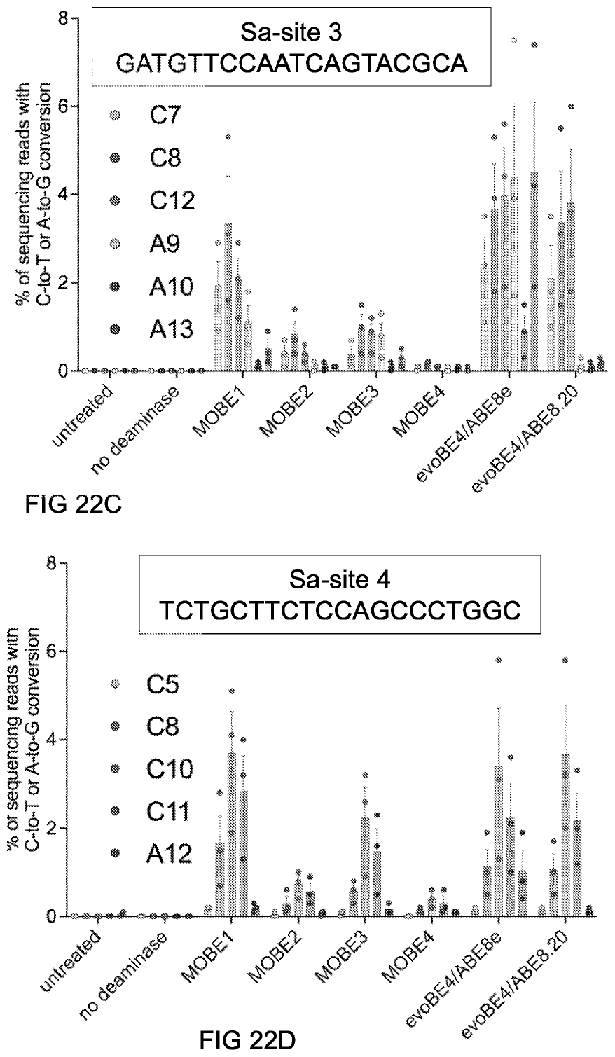

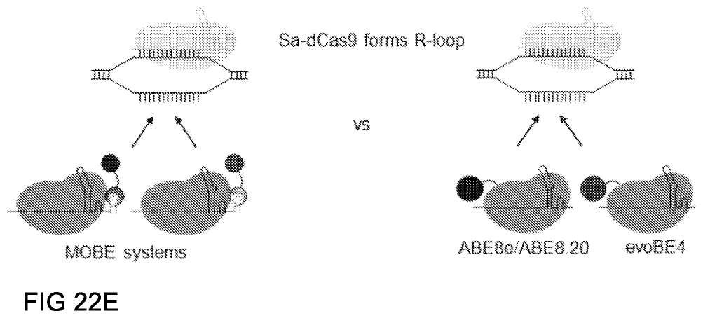

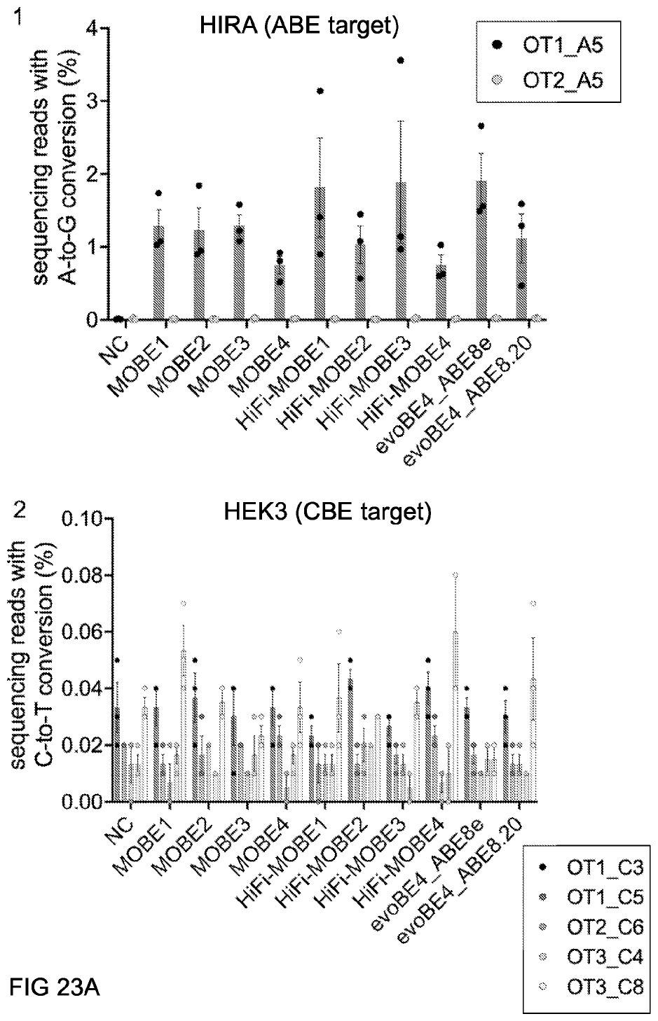

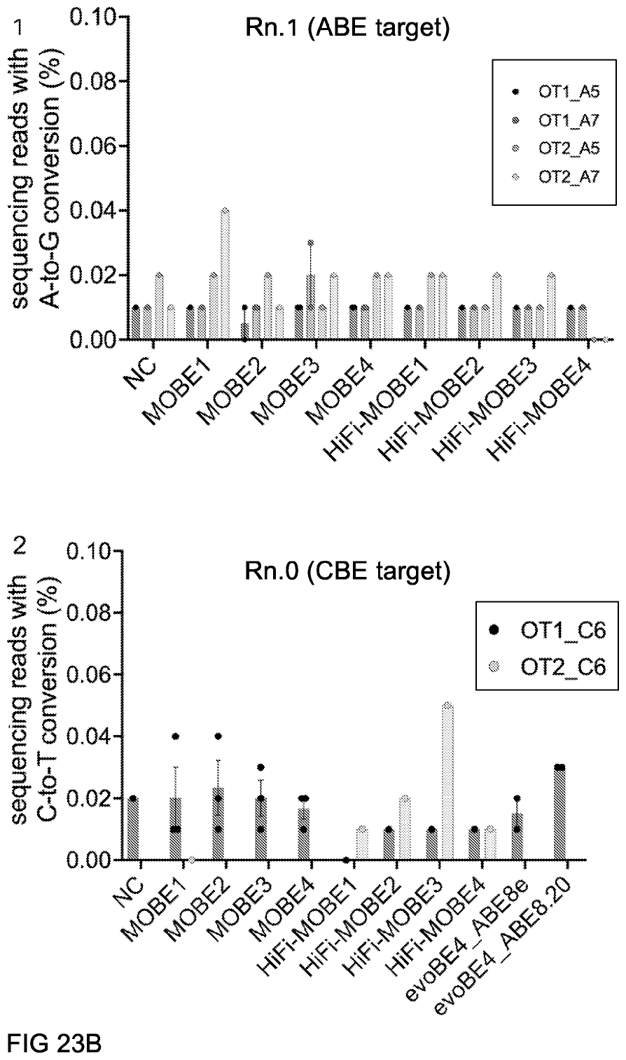

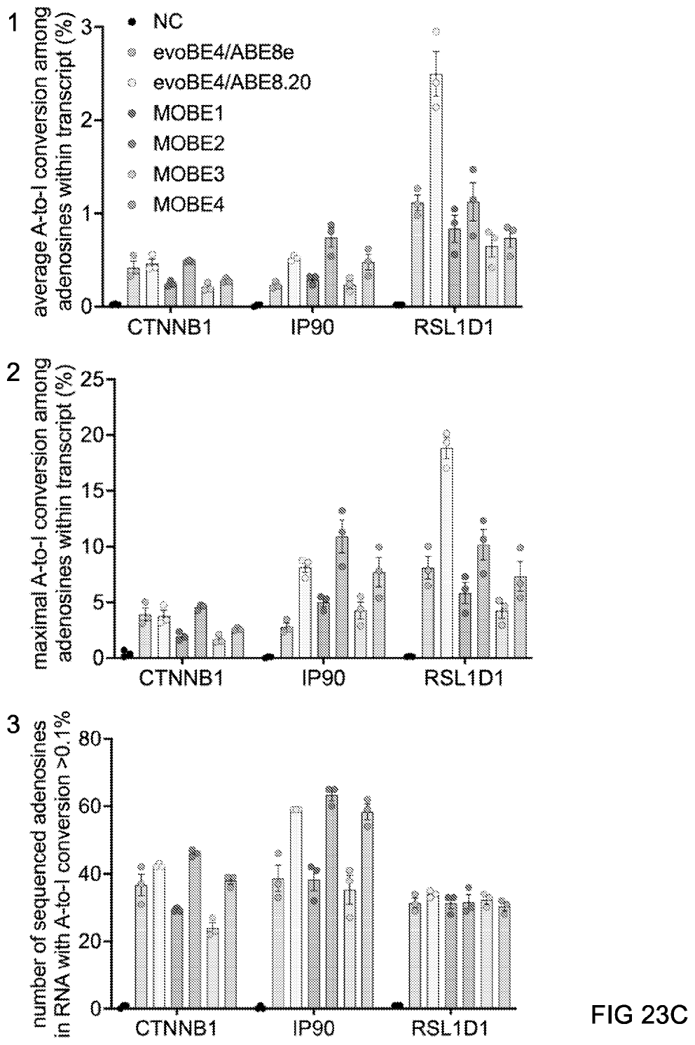

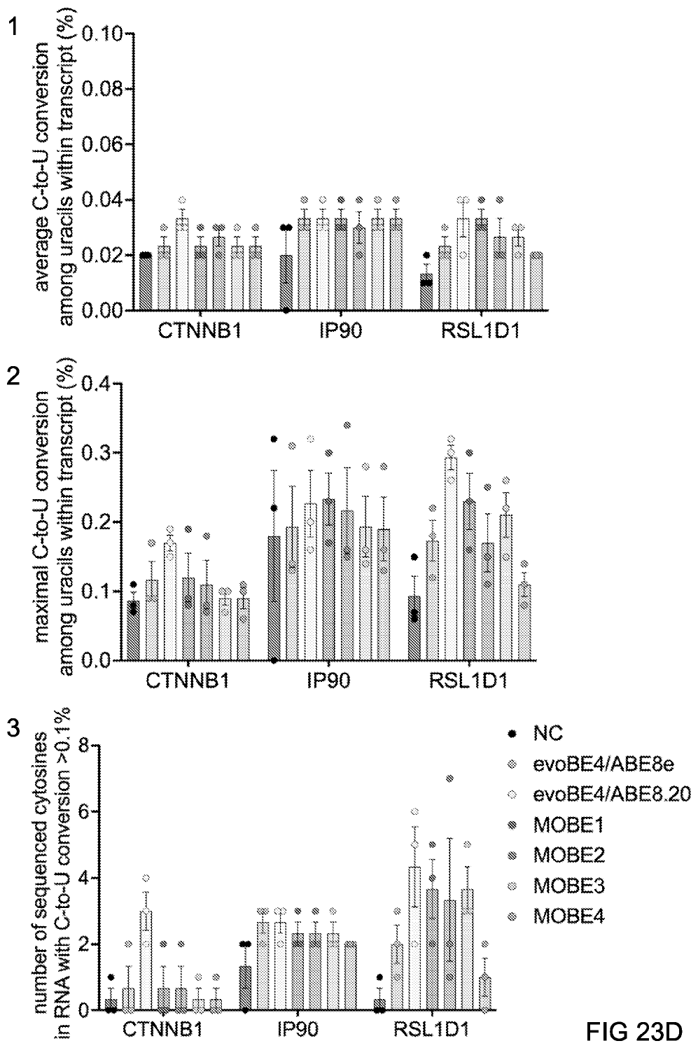

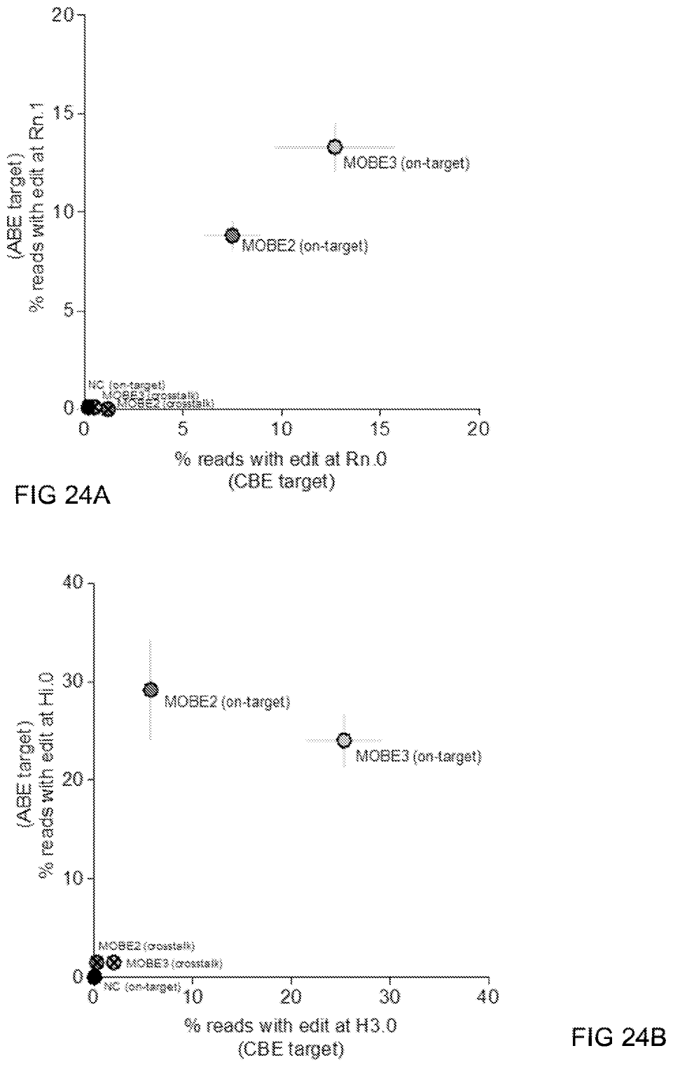

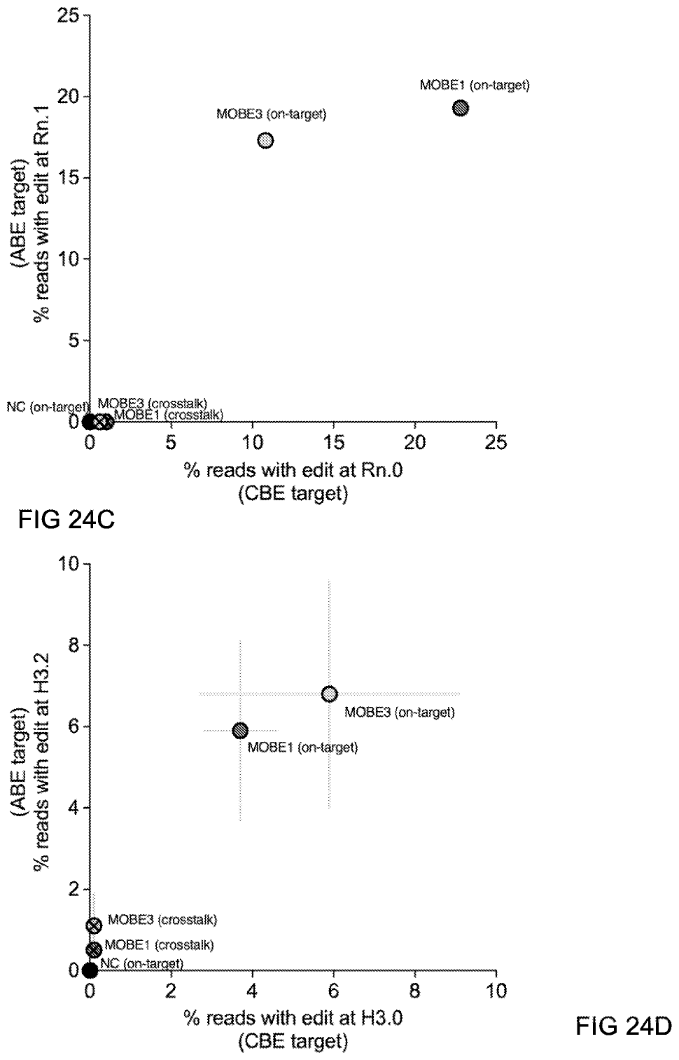

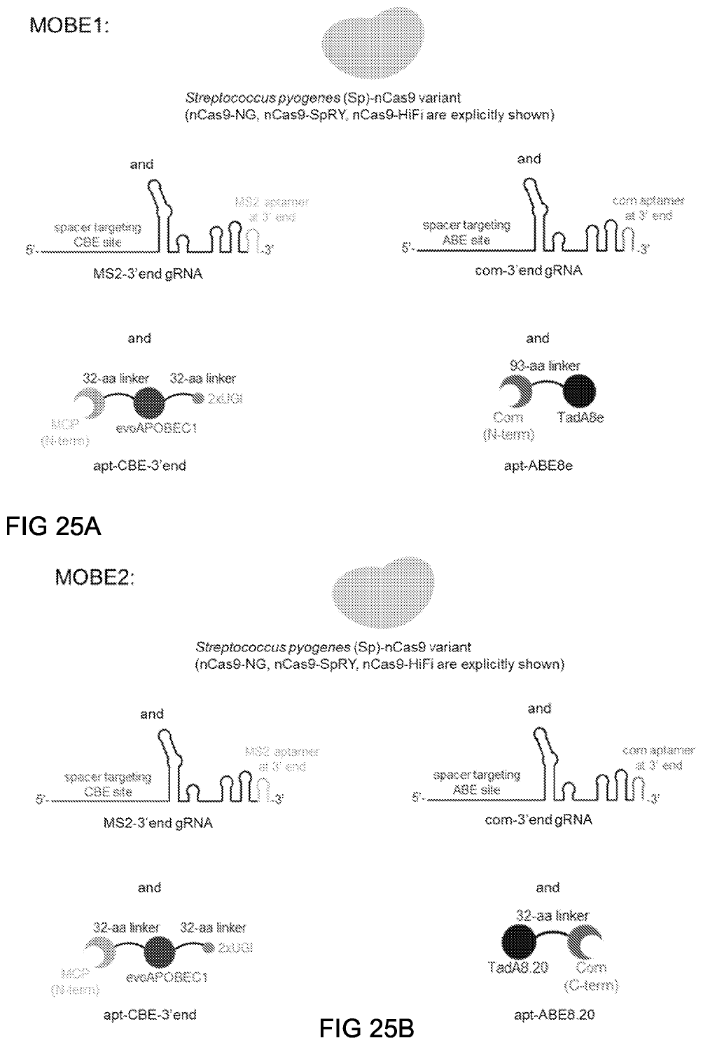

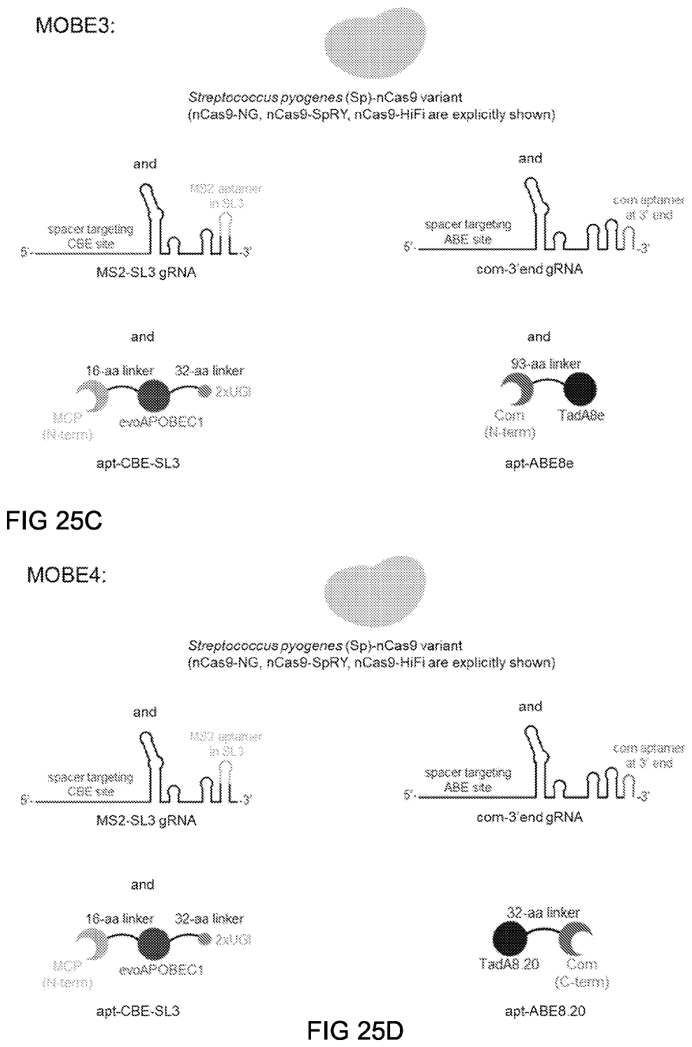

Plotted are the percent of total DNA sequencing reads with A⋅T edited to G⋅C at the ABE target (y-axis, solid circles), C⋅G edited to T⋅A at the ABE target (y-axis, circles with X inset), C⋅G edited to T⋅A at the CBE target (x-axis, solid circles), and A⋅T edited to G⋅C at the CBE target (x-axis, circles with X inset). Dot plots and error bars represent the average and sem for n=3 biological replicates. FIGS. 19D-19F. Plotted are the percent of total DNA sequencing reads with A⋅T edited to G⋅C at the ABE target and C⋅G edited to T⋅A at the CBE target (on-target efficiencies only). Bar graphs and error bars represent the average and sem for n=3 biological replicates (each replicate is marked individually). Negative control (NC) samples are also shown, in which cells were not transfected with any plasmid.

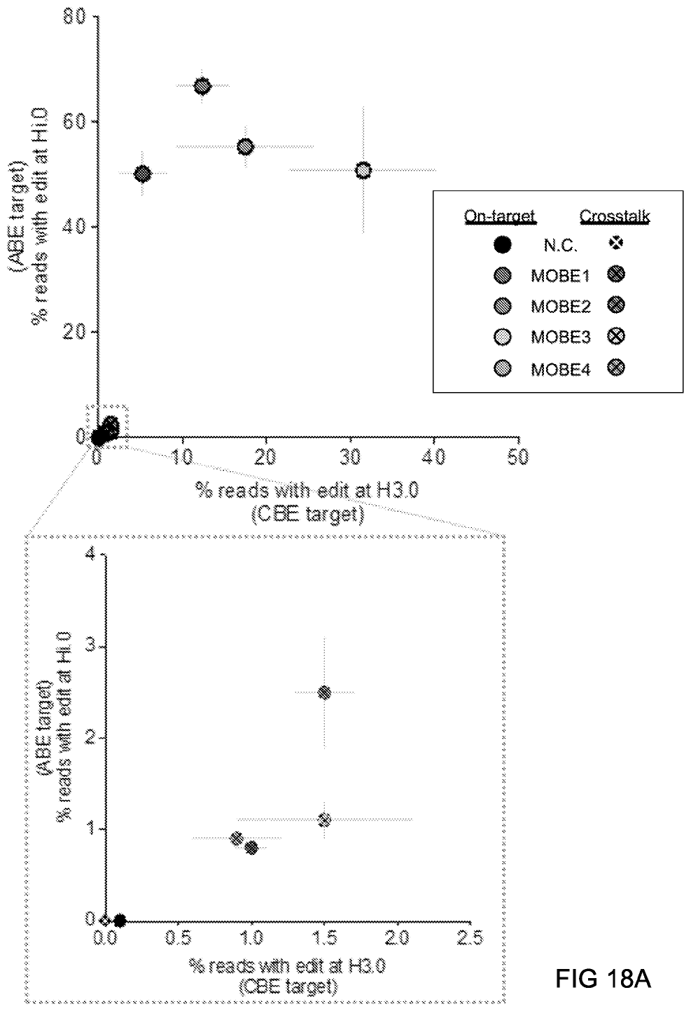

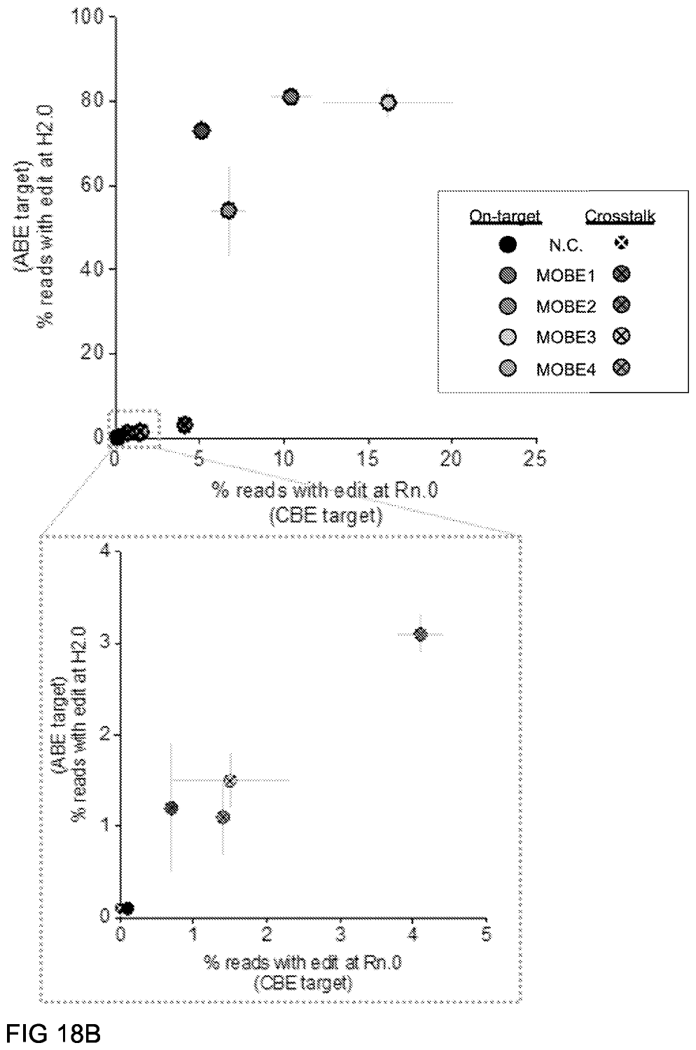

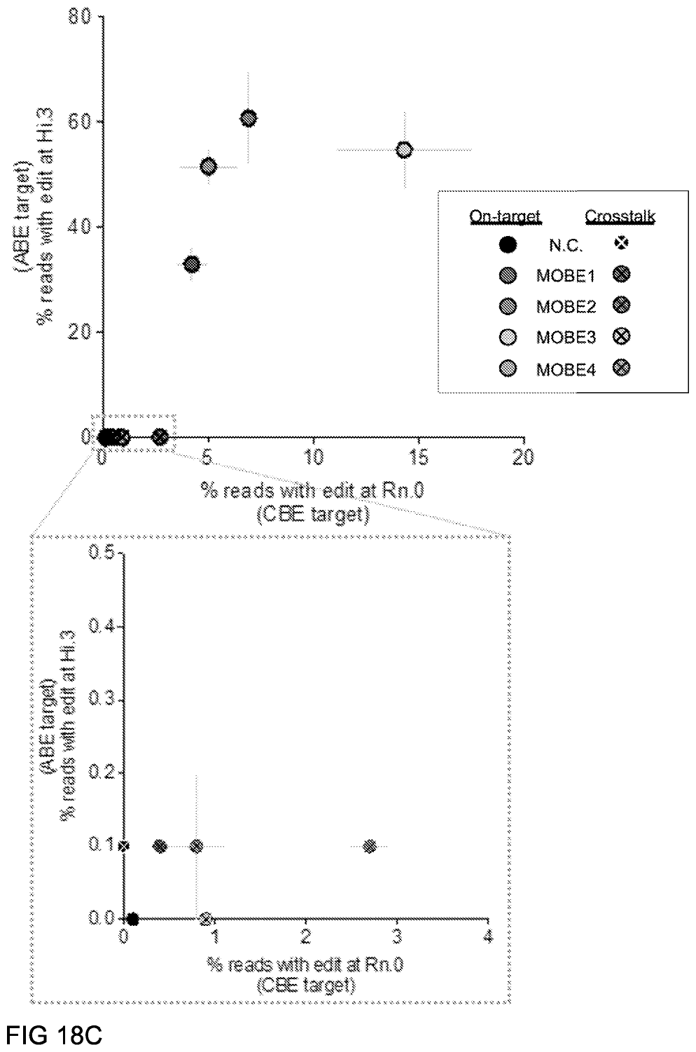

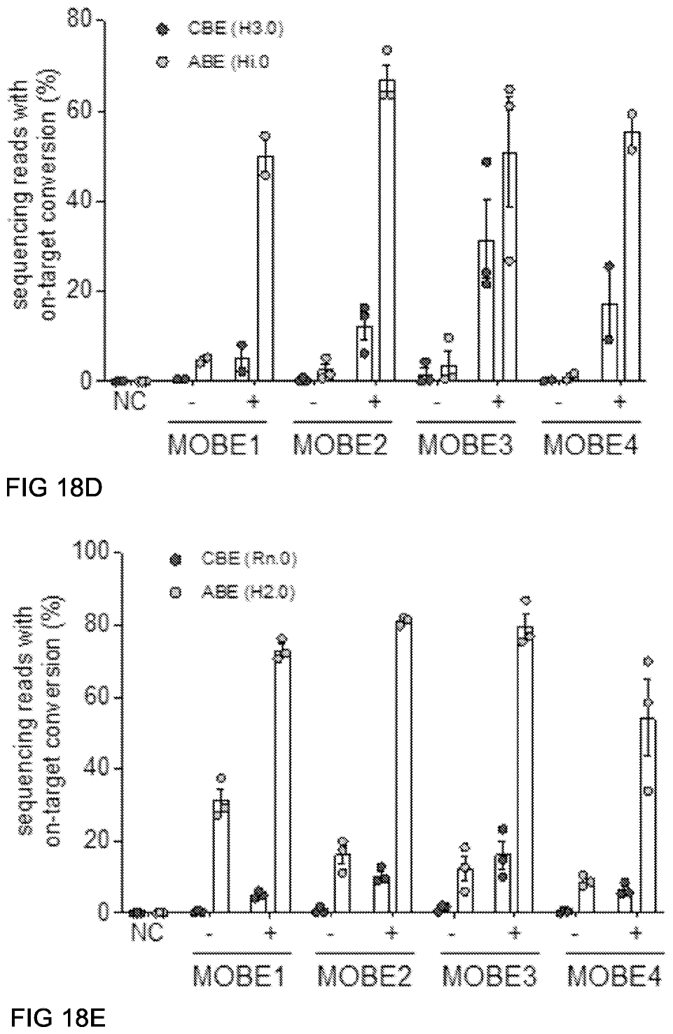

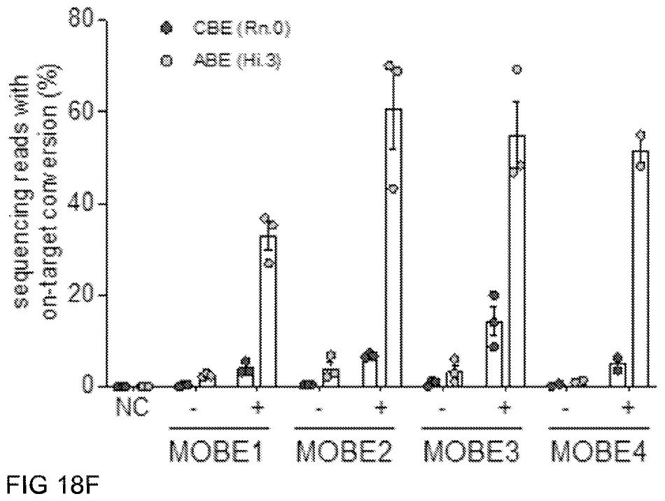

FIGS. 20A-20B. Orthogonality scores and increases in on-target editing efficiencies after enrichment with the fluorescent reporter. FIG. 20A. Cells were treated as previously described in FIGS. 18A-18F. Plotted are the on-target enrichment values, which are the log 2 of the percent of total DNA sequencing reads with C⋅G edited to T⋅A at the CBE target of the “enriched” cells divided by that of the “unenriched” cells, or the A⋅T to G⋅C editing efficiency equivalent. Each dot represents the average of n=3 biological replicates for a given protospacer (each MOBE has twelve individual enrich values). Bar graphs and error bars represent the average and standard deviation of the twelve dot plots for a given MOBE system. FIG. 20B. Orthogonality scores for the MOBE1-4 systems at all six protospacer combinations tested after enrichment using the fluorescent reporter. Plotted are the log 2 of the “CBE orthogonality scores”, which were defined for a given protospacer combination as the percent of total DNA sequencing reads with C⋅G edited to T⋅A at the CBE target divided by the percent of total DNA sequencing reads with C⋅G edited to T⋅A at the ABE target. The “ABE orthogonality scores” are the A⋅T to G⋅C editing efficiency equivalent. Dot plots and error bars represent the average and standard deviation for n=3 biological replicates.

FIGS. 21A-21B. Compatibility of MOBE systems with additional Sp-nCas9 variants. HEK293T cells were transfected with plasmids encoding tandem CP-deaminase fusions, both gRNA-aptamers, and either nCas9-NG-P2A-mCherry, HiFi-nCas9-P2A-mCherry, or SpRY-nCas9-P2A-mCherry. Cells were then lysed after 72 hours, genomic loci of interest were amplified, and analyzed by NGS. Plotted are the percent of total DNA sequencing reads with A⋅T edited to G⋅C at the ABE target (y-axis, filled circles, squares, and diamonds), C⋅G edited to T⋅A at the ABE target (y-axis, circles, squares, and diamonds with X inset), C⋅G edited to T⋅A at the CBE target (x-axis, filled circles, squares, and diamonds), and A⋅T edited to G⋅C at the CBE target (x-axis, circles, squares, and diamonds with X inset). Dot plots and error bars represent the average and SEM for n=3 to 4 biological replicates. Negative control (NC) samples are also shown, in which cells were treated identically but transfected with a non-targeting gRNA. Editing was evaluated at the HIRA.0/HEK3.0 protospacer combination (FIG. 21A) and the RNF2 single amplicon protospacer combination (FIG. 21B).

FIGS. 22A-22E. Evaluation of gRNA-independent off-target DNA editing by MOBEs compared to parental systems. FIGS. 22A-22D. HEK293T cells were transfected with plasmids encoding nCas9-NG-P2A-mCherry, tandem CP-deaminase fusions, and both gRNA-aptamers (for the MOBE systems), or plasmids encoding the parental evoBE4-NG, ABE8e-NG or ABE8.20-NG, and both unmodified gRNAs (for the evoBE4-NG/ABE8e-NG and evoBE4-NG/ABE8.20-NG combinations), as well as Sa-dCas9 and Sa-gRNA targeted to the four protospacer sequences shown. Cells were then lysed after 72 hours, genomic loci of interest were amplified, and analyzed by NGS. Plotted are the percent of total DNA sequencing reads with C⋅G edited to T⋅A at the target Cs indicates, and A⋅T edited to G⋅C at the target As indicated. Bar graphs represent the average, and error bars represent the SEM for n=3 biological replicates, with each replicate marked individually. Untreated samples are also shown, in which cells were non-transfected. Negative control (no deaminase) samples are also shown, in which cells were treated identically as the MOBE systems, but with omission of the tandem CP-deaminase fusion plasmid. FIG. 22E. Schematic of the orthogonal R-loop assay used to evaluate gRNA-independent off-target editing. The Sa-dCas9:gRNA complex binds to a genomic locus of interest and exposes a stretch of ssDNA via the formation of an R-loop. The deaminase components of additional BE complexes within the cell (either the MOBE systems or the parental ABE/CBE complexes) can access the exposed ssDNA and edit Cs or As within this region, which is quantified with NGS. Figure discloses SEQ ID NOS: 25-28, respectively, in order of appearance.

FIGS. 23A-23D. Evaluation of gRNA-dependent off-target DNA editing and RNA off-target editing by MOBEs compared to parental systems. FIGS. 23A-23B. HEK293T cells were transfected with plasmids encoding nCas9-NG-P2A-mCherry, tandem CP-deaminase fusions, and both gRNA-aptamers (for the MOBE systems), or plasmids encoding the parental evoBE4-NG, ABE8e-NG or ABE8.20-NG, and both unmodified gRNAs (for the evoBE4-NG/ABE8e-NG and evoBE4-NG/ABE8.20-NG combinations), targeted to the HIRA.0/HEK3.0 protospacer combination (FIG. 23A) or the RNF2 single amplicon protospacer combination (FIG. 23B). Cells were then lysed after 72 hours, genomic loci of interest were amplified, and analyzed by NGS. Plotted are the percent of total DNA sequencing reads with A⋅T edited to G⋅C at the top two putative off-target sites for the ABE protospacers, and C⋅G edited to T⋅A at the top two putative off-target sites for the CBE protospacers. Bar graphs represent the average, and error bars represent the SEM for n=3 biological replicates, with each replicate marked individually. Negative control (NC) samples are also shown, in which cells were treated identically but transfected with a non-targeting gRNA. FIGS. 23C-23D. HEK293T cells were transfected with plasmids encoding nCas9-NG-P2A-mCherry, tandem CP-deaminase fusions, and both gRNA-aptamers (for the MOBE systems), or plasmids encoding the parental evoBE4-NG, ABE8e-NG or ABE8.20-NG, and both unmodified gRNAs (for the evoBE4-NG/ABE8e-NG and evoBE4-NG/ABE8.20-NG combinations). After 48 hours, the total RNA was extracted, and the mRNA was reverse-transcribed into cDNA. The three transcriptomic sites of interest (CTNNB1, IP90, and RSL1D1) were sequenced with NGS. Plotted are the average A to I (FIG. 23C) or C to U (FIG. 23D) conversion among all As or Cs within the transcript (left graphs), the maximal A to I (FIG. 23C) or C to U (FIG. 23D) conversion among all As or Cs within the transcript (middle graphs), and the number of As or Cs within the transcripts with A to I (FIG. 23C) or C to U (FIG. 23D) conversions greater than 0.1% (right graphs). Bar graphs represent the average, and error bars represent the SEM for n=3 biological replicates, with each replicate marked individually. Negative control (NC) samples are also shown, in which cells were treated identically but transfected with a non-targeting gRNA.

FIGS. 24A-24D. MOBEs are compatible with additional cell types. FIGS. 24A-24B. HeLa cells were transfected with plasmids encoding nCas9-NG-P2A-mCherry, tandem CP-deaminase fusions, both gRNA-aptamers targeting endogenous loci [the RNF2 single amplicon protospacer combination (FIG. 24A), and the HIRA.0/HEK3.0 protospacer combination (FIG. 24B)], and the 2×-dead-GFP reporter. After 72 hours, the population of GFP+/mCherry+“enriched” cells were collected by FACS. Genomic loci of interest were then amplified and analyzed by NGS. Plotted are the percent of total DNA sequencing reads with A⋅T edited to G⋅C at the ABE target (y-axis, filled circles), C⋅G edited to T⋅A at the ABE target (y-axis, circles with X inset), C⋅G edited to T⋅A at the CBE target (x-axis, filled circles), and A⋅T edited to G⋅C at the CBE target (x-axis, circles with X inset). Dot plots and error bars represent the average and standard deviation for n=3 biological replicates. Negative control (NC) samples are also shown, in which cells were treated identically but transfected with a non-targeting gRNA. FIGS. 24C-24D. SH-Sy5Y cells were treated identically, but with gRNA-aptamers targeting the RNF2 (FIG. 24C) and HEK3 (FIG. 24D) single amplicon protospacer combinations.

FIGS. 25A-25D illustrate each MOBE construct. FIG. 25A illustrates MOBE1, Streptococcus pyogenes (Sp)-nCas9 variant, of which nCas9-NG, nCas9-SpRY, and nCas9-HiFi are explicitly shown. FIG. 25B illustrates MOBE2, Streptococcus pyogenes (Sp)-nCas9 variant, of which nCas9-NG, nCas9-SpRY, and nCas9-HiFi are explicitly shown. FIG. 25C illustrates MOBE3, Streptococcus pyogenes (Sp)-nCas9 variant, of which nCas9-NG, nCas9-SpRY, and nCas9-HiFi are explicitly shown. FIG. 25D illustrates MOBE4, Streptococcus pyogenes (Sp)-nCas9 variant, of which nCas9-NG, nCas9-SpRY, and nCas9-HiFi are explicitly shown.

Additional advantages of the present disclosure are set forth in part in the description which follows, and in part could be obvious from the description, or can be learned by practice of the disclosure. The advantages of the disclosure could be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present disclosure provides four multiplexed orthogonal base editor (MOBE) systems derived from the CRISPR/Cas9 protein. The MOBE systems described herein enable the simultaneous introduction of C⋅G to T⋅A and A⋅T to G⋅C point mutations at distinct genomic loci in living cells, with high efficiency and precision and with minimal crosstalk. These systems are derived from “base editor” technologies, in which Cas9 is catalytically impaired and fused to an enzyme that performs DNA nucleobase chemistry. There are currently two major classes of base editors that use cytosine and adenine deamination chemistries to catalyze the conversion of C⋅G base pairs to T⋅A (CBEs), and A⋅T base pairs to G⋅C (ABEs), respectively. The two current systems cannot be used together, as there is no way to independently program a CBE to one genomic loci and an ABE to another genomic loci.

However, the MOBE systems described herein allow this by tethering the deaminase enzymes to the gRNA of the CRISPR/Cas9 system. In certain embodiments, the MOBE system of the present disclosure comprises a combination of aptamer-based Cytidine-BE system and an aptamer-based Adenosine-BE system. In certain embodiments, each aptamer-based BE system comprises aptamer-gRNA constructs that are combined with corresponding coat protein-deaminase fusions.

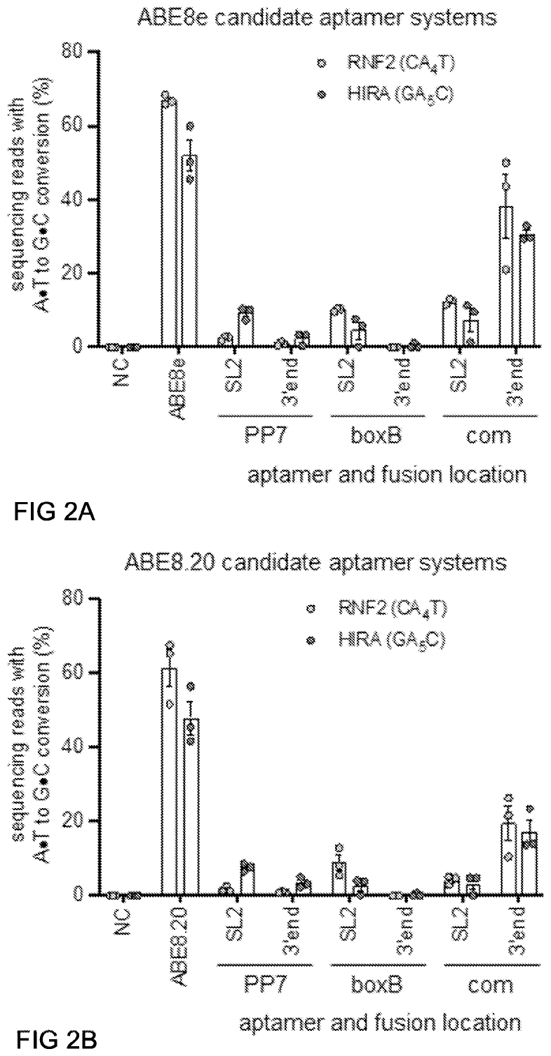

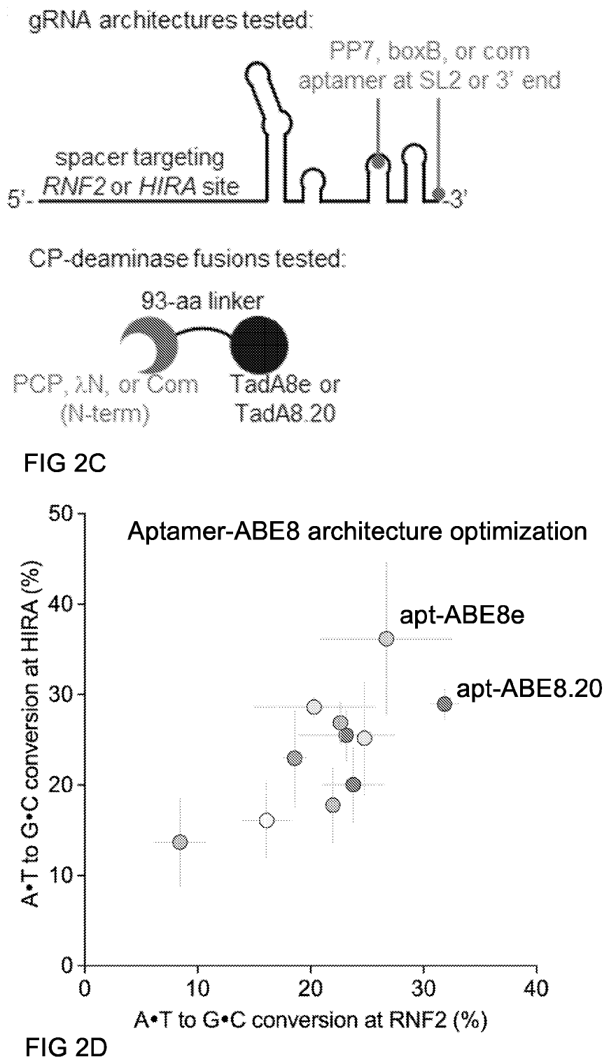

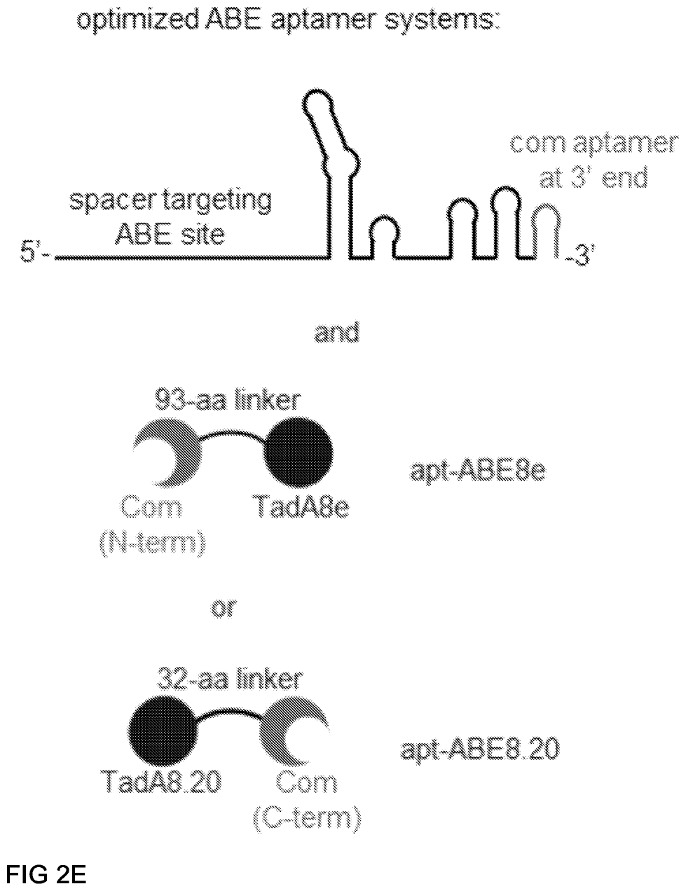

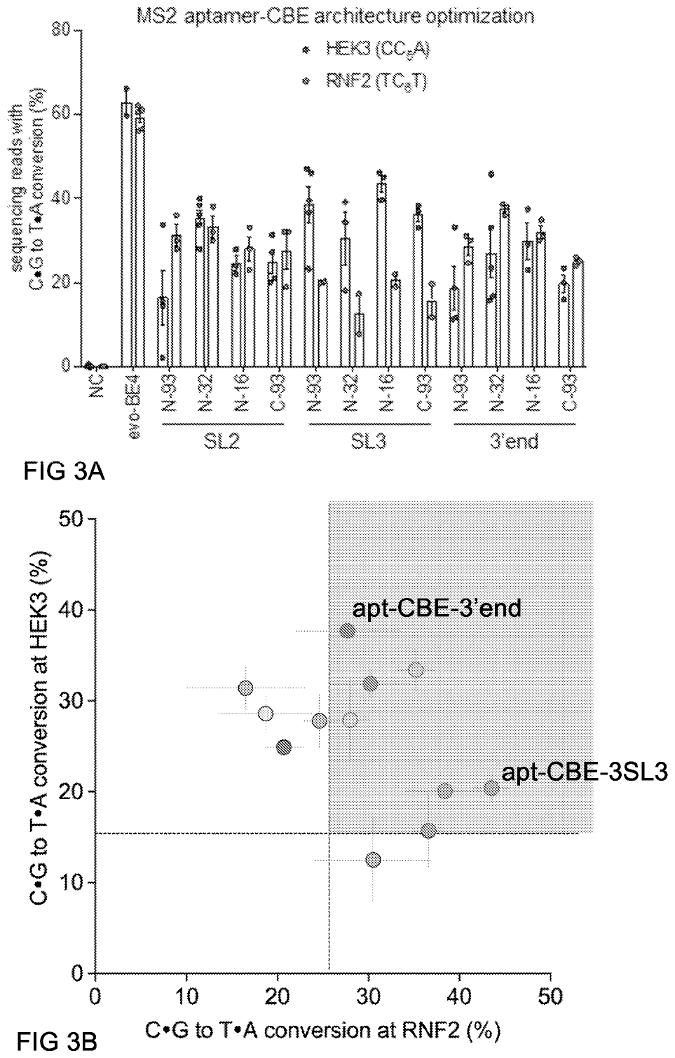

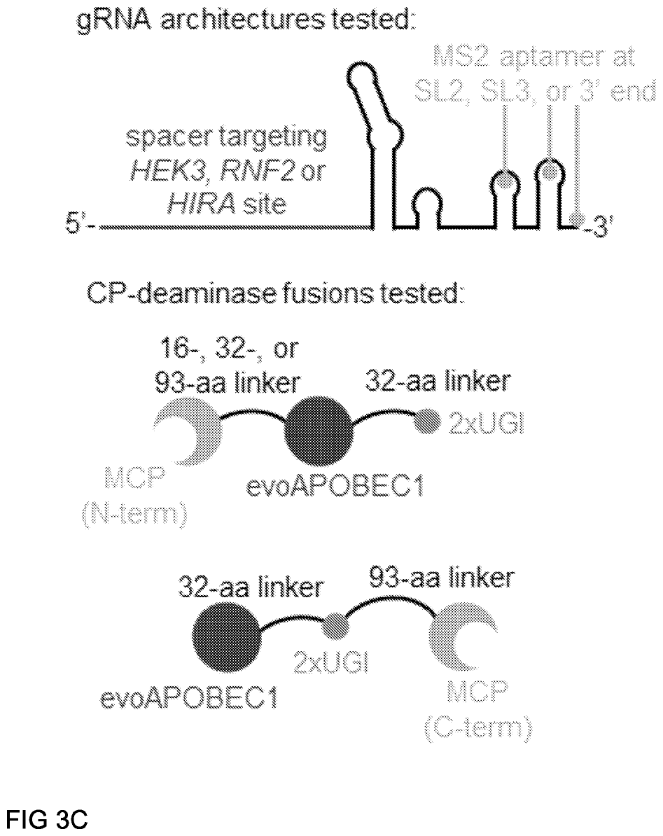

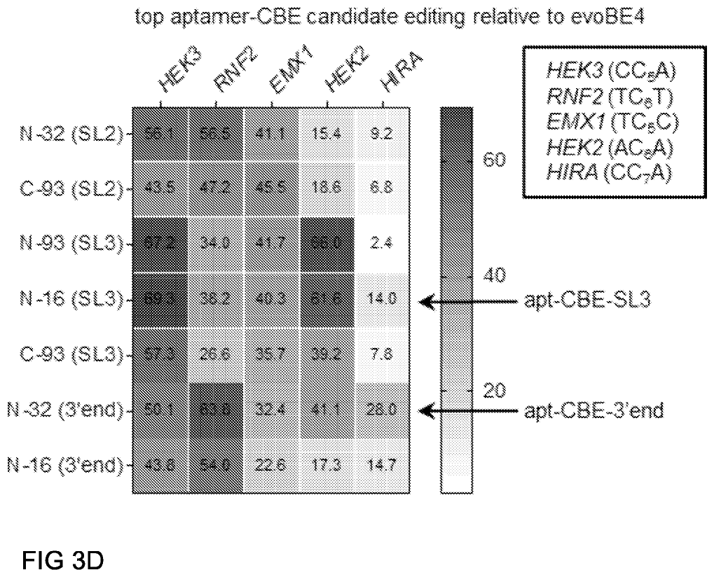

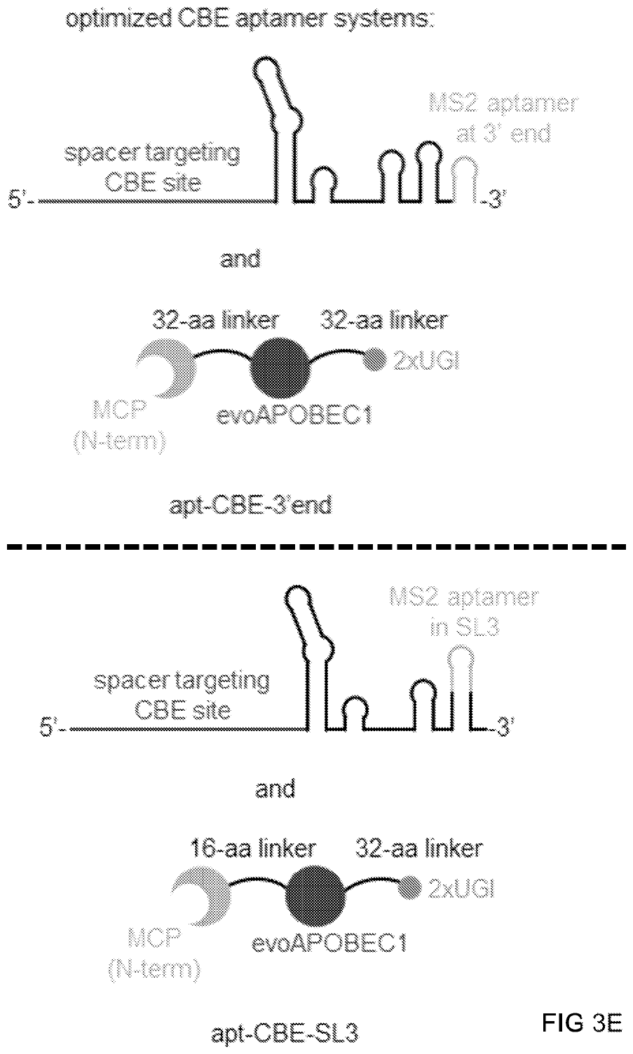

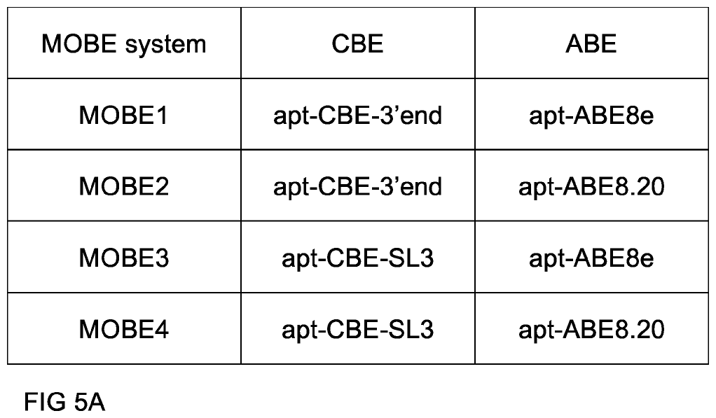

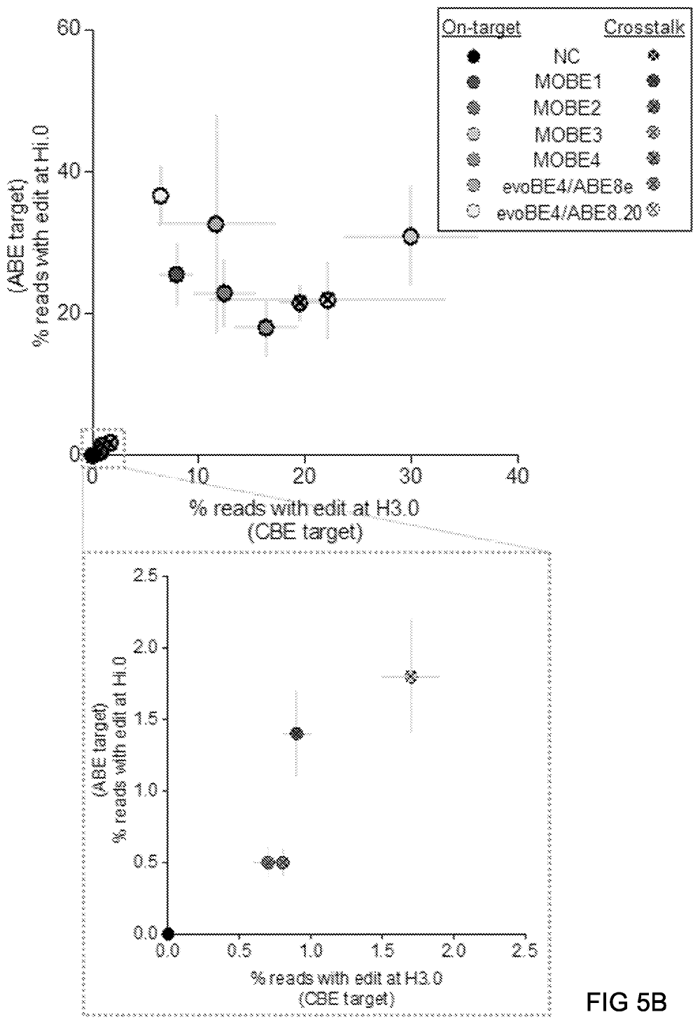

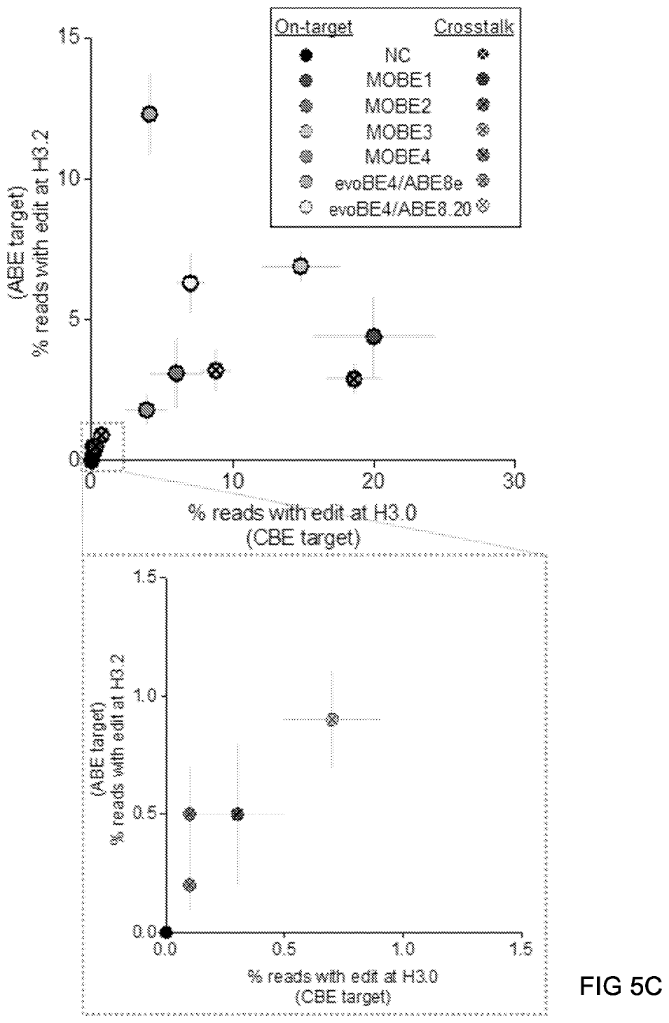

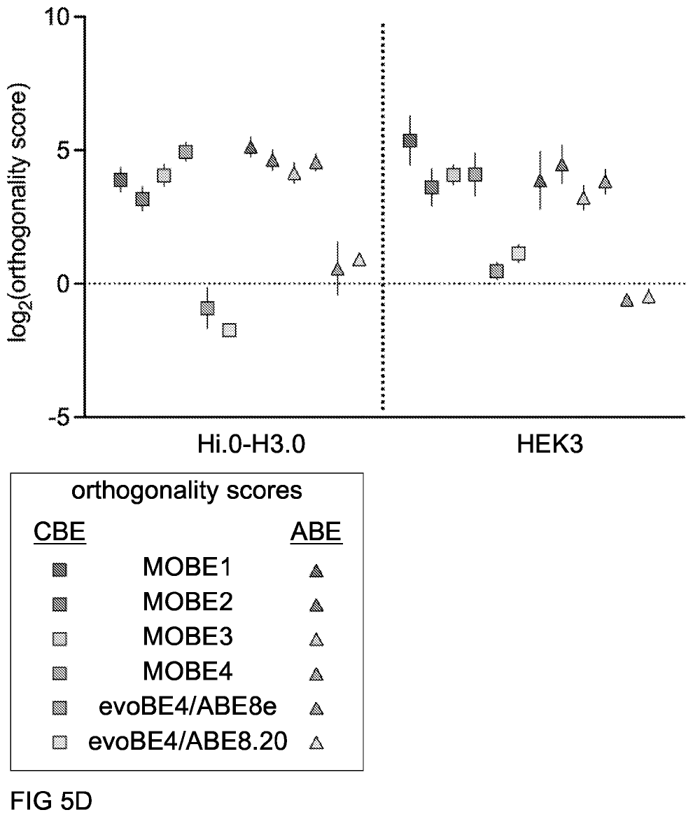

In certain embodiments, the present disclosure provides four MOBE systems, namely, MOBE1, MOBE2, MOBE3, and MOBE4. For instance, MOBE1: Sp-nCas9 variant with apt-CBE-3′end is shown in FIG. 3E (left) and with apt-ABE8e is shown in FIG. 2E (left, top); MOBE2: Sp-nCas9 variant with apt-CBE-3′end is shown in FIG. 3E, (left), and with apt-ABE8.20 is shown in FIG. 2E (left, bottom); MOBE3: Sp-nCas9 variant with apt-CBE-SL3 is shown in FIG. 3E (right) and with apt-ABE8e is shown in FIG. 2E (left, top); and MOBE4: Sp-nCas9 variant with apt-CBE-SL3 is shown in FIG. 3E (right) and with apt-ABE8.20 is shown in FIG. 2E (left, bottom). Moreover, FIG. 5A summarizes the CBE and ABE of each MOBE, and each construct of each MOBE is presented in FIGS. 25A-25D. The sequence information of each MOBE, as well as each component of each MOBE, is provided in the following Table.

Table-MOBE sequences

| aptamer- | |
|---|---|
| gRNA fusion | sequence |
| Spy-gRNA | [[N20]guuuuagagcuagaaauagcaaguuaaaauaaggcuaguccguuaucaacuugaaaaaguggcaccgagucggugc |
| backbone | (SEQ ID NO: 1) |
| PP7-TL | [[N20]]guuuuagagcuaCCUAAGGAGUUUAUAUGGAAACCCUUAGGuagcaaguuaaaauaaggcuaguccguuaucaacuug |
| | aaaaaguggcaccgagucggugc (SEQ ID NO: 2) |
| PP7-SL2 | [[N20]]guuuuagagcuagaaauagcaaguuaaaauaaggcuaguccguuaucaacuuCCUAAGGAGUUUAUAUGGAAACCCUUA |
| | GGaaguggcaccgagueggugc (SEQ ID NO: 3) |
| PP7-3′end | [[N20]]guuuuagagcuagaaauagcaaguuaaaauaaggcuaguccguuaucaacuugaaaaaguggcaccgagucggugcAAC |
| | AUAAGGAGUUUAUAUGGAAACCCUUAUG (SEQ ID NO: 4) |
| boxB-SL2 | [[N20]]guuuuagagcuagaaauagcaaguuaaaauaaggcuaguccguuaucaacuuGGGCCCUGAAGAAGGGCCCaaguggca |
| | ccgagucggugc (SEQ ID NO: 5) |
| boxB-3'end | [[N20]]guuuuagagcuagaaauagcaaguuaaaauaaggcuaguccguuaucaacuugaaaaaguggcaccgagucggugcGCG |
| | GGCCCUGAAGAAGGGCCC (SEQ ID NO: 6) |
| com-SL2 | [[N20]]guuuuagagcuagaaauagcaaguuaaaauaaggcuaguccguuaucaacuuCUGAAUGCCUGCGAGCAUCaaguggca |
| | ccgaguegguge (SEQ ID NO: 7) |
| com-3′end | [[N20]]guuuuagagcuagaaauagcaaguuaaaauaaggcuaguccguuaucaacuugaaaaaguggcaccgagucggugcCUG |
| (used in all | AAUGCCUGCGAGCAUC (SEQ ID NO: 8) |
| MOBEs) | |
| MS2-TL | [[N20]]guuuuagagcuaGGCCAACAUGAGGAUCACCCAUGUCUGCAGGGCCuagcaaguuaaaauaaggcuaguccguuau |
| | caacuugaaaaaguggcaccgagueggugc (SEQ ID NO: 9) |
| MS2-SL2 | [[N20]]guuuuagagcuagaaauagcaaguuaaaauaaggcuaguccguuaucaacuuCCAACAUGAGGAUCACCCAUGUCUGC |
| | AGGGaaguggcaccgagucggugc (SEQ ID NO: 10) |
| MS2-SL3 | [[N20]]guuuuagagcuagaaauagcaaguuaaaauaaggcuaguccguuaucaacuugaaaaaguggcaccgGCACAUGAGGAU |
| (used in | CACCCAUGUGCcggugc (SEQ ID NO: 11) |
| MOBE3 and | |
| MOBE4) | |
| MS2-3′end | [[N20]]guuuuagagcuagaaauagcaaguuaaaauaaggcuaguccguuaucaacuugaaaaaguggcaccgagucggugcGCG |
| (used in | CACAUGAGGAUCACCCAUGUGC (SEQ ID NO: 12) |
| MOBE1 and | |
| MOBE2) | |

CAPS = inserted sequence; underlined=core aptamer

| | | | Plasmid | |
|---|---|---|---|---|
| | Nickname | Description | # | Sequence |
| [symbol] | nCas9-NG | nCas9(D10A)- | QTC24 | MKRTADGSEFESPKKKRKVDKKYSIGLAIGTNSVGWAVITDEYKVPSKKFKVL |
| deam- | | NG_P2A- | | GNTDRHSIKKNLIGALLFDSGETAEATRLKRTARRRYTRRKNRICYLQEIFSNE |
| inases: | | mCherry | | MAKVDDSFFHRLEESFLVEEDKKHERHPIFGNIVDEVAYHEKYPTIYHLRKKL |
| | | | | VDSTDKADLRLIYLALAHMIKFRGHFLIEGDLNPDNSDVDKLFIQLVQTYNQLF |
| | | | | EENPINASGVDAKAILSARLSKSRRLENLIAQLPGEKKNGLFGNLIALSLGLTP |
| | | | | NFKSNFDLAEDAKLQLSKDTYDDDLDNLLAQIGDQYADLFLAAKNLSDAILLSD |
| | | | | ILRVNTEITKAPLSASMIKRYDEHHQDLTLLKALVRQQLPEKYKEIFFDQSKNG |
| | | | | YAGYIDGGASQEEFYKFIKPILEKMDGTEELLVKLNREDLLRKQRTFDNGSIPH |
| | | | | QIHLGELHAILRRQEDFYPFLKDNREKIEKILTFRIPYYVGPLARGNSRFAWMT |
| | | | | RKSEETITPWNFEEVVDKGASAQSFIERMTNFDKNLPNEKVLPKHSLLYEYFT |
| | | | | VYNELTKVKYVTEGMRKPAFLSGEQKKAIVDLLFKTNRKVTVKQLKEDYFKKI |
| | | | | ECFDSVEISGVEDRFNASLGTYHDLLKIIKDKDFLDNEENEDILEDIVLTLTLFE |
| | | | | DREMIEERLKTYAHLFDDKVMKQLKRRRYTGWGRLSRKLINGIRDKQSGKTIL |
| | | | | DFLKSDGFANRNFMQLIHDDSLTFKEDIQKAQVSGQGDSLHEHIANLAGSPAI |
| | | | | KKGILQTVKVVDELVKVMGRHKPENIVIEMARENQTTQKGQKNSRERMKRIE |
| | | | | EGIKELGSQILKEHPVENTQLQNEKLYLYYLQNGRDMYVDQELDINRLSDYDV |
| | | | | DHIVPQSFLKDDSIDNKVLTRSDKNRGKSDNVPSEEVVKKMKNYWRQLLNAK |
| | | | | LITQRKFDNLTKAERGGLSELDKAGFIKRQLVETRQITKHVAQILDSRMNTKYD |
| | | | | ENDKLIREVKVITLKSKLVSDFRKDFQFYKVREINNYHHAHDAYLNAVVGTALI |
| | | | | KKYPKLESEFVYGDYKVYDVRKMIAKSEQEIGKATAKYFFYSNIMNFFKTEITL |
| | | | | ANGEIRKRPLIETNGETGEIVWDKGRDFATVRKVLSMPQVNIVKKTEVQTGGF |
| | | | | SKESIRPKRNSDKLIARKKDWDPKKYGGFVSPTVAYSVLVVAKVEKGKSKKL |
| | | | | KSVKELLGITIMERSSFEKNPIDFLEAKGYKEVKKDLIIKLPKYSLFELENGRKR |
| | | | | MLASARFLQKGNELALPSKYVNFLYLASHYEKLKGSPEDNEQKQLFVEQHKH |
| | | | | YLDEIIEQISEFSKRVILADANLDKVLSAYNKHRDKPIREQAENIIHLFTLTNLG |
| | | | | APRAFKYFDTTIDRKVYRSTKEVLDATLIHQSITGLYETRIDLSQLGGDSGGSKR |
| | | | | TADGSEFEPKKKRKVGSGATNFSLLKQAGDVEENPGPMVSKGEEDNMAIIKE |
| | | | | FMRFKVHMEGSVNGHEFEIEGEGEGRPYEGTQTAKLKVTKGGPLPFAWDIL |
| | | | | SPQFMYGSKAYVKHPADIPDYLKLSFPEGFKWERVMNFEDGGVVTVTQDSS |
| | | | | LQDGEFIYKVKLRGTNFPSDGPVMQKKTMGWEASSERMYPEDGALKGEIKQ |
| | | | | RLKLKDGGHYDAEVKTTYKAKKPVQLPGAYNVNIKLDITSHNEDYTIVEQYER |
| | | | | AEGRHSTGGMDELYKSGGSPKKKRKV (SEQ ID NO: 13) |
| | nCas9- | nCas9(D10A)- | QTC290 | MKRTADGSEFESPKKKRKVDKKYSIGLAIGTNSVGWAVITDEYKVPSKKFKVL |
| | SpRY | SpRY_P2A- | | GNTDRHSIKKNLIGALLFDSGETAERTRLKRTARRRYTRRKNRICYLQEIFSNE |
| | | mCherry | | MAKVDDSFFHRLEESFLVEEDKKHERHPIFGNIVDEVAYHEKYPTIYHLRKKL |
| | | | | VDSTDKADLRLIYLALAHMIKFRGHFLIEGDLNPDNSDVDKLFIQLVQTYNQLF |
| | | | | EENPINASGYDAKAILSARLSKSRRLENLIAQLPGEKKNGLFGNLIALSLGLTP |
| | | | | NFKSNFDLAEDAKLQLSKDTYDDDLDNLLAQIGDQYADLFLAAKNLSDAILLSD |
| | | | | ILRVNTEITKAPLSASMIKRYDEHHQDLTLLKALVRQQLPEKYKEIFFDQSKNG |
| | | | | YAGYIDGGASQEEFYKFIKPILEKMDGTEELLVKLNREDLLRKQRTFDNGSIPH |
| | | | | QIHLGELHAILRRQEDFYPFLKDNREKIEKILTFRIPYYYGPLARGNSRFAWMT |
| | | | | RKSEETITPWNFEEVVDKGASAQSFIERMTNFDKNLPNEKVLPKHSLLYEYFT |
| | | | | VYNELTKVKYVTEGMRKPAFLSGEQKKAIVDLLFKTNRKVTVKQLKEDYFKKI |
| | | | | ECFDSVEISGVEDRFNASLGTYHDLLKIIKDKDFLDNEENEDILEDIVLTLTLFE |
| | | | | DREMIEERLKTYAHLFDDKVMKQLKRRRYTGWGRLSRKLINGIRDKQSGKTIL |
| | | | | DFLKSDGFANRNFMQLIHDDSLTFKEDIQKAQVSGQGDSLHEHIANLAGSPAI |
| | | | | KKGILQTVKVVDELVKVMGRHKPENIVIEMARENQTTQKGQKNSRERMKRIE |
| | | | | EGIKELGSQILKEHPVENTQLQNEKLYLYYLQNGRDMYVDQELDINRLSDYDV |
| | | | | DHIVPQSFLKDDSIDNKVLTRSDKNRGKSDNVPSEEVVKKMKNYWRQLLNAK |
| | | | | LITQRKFDNLTKAERGGLSELDKAGFIKRQLVETRQITKHVAQILDSRMNTKYD |
| | | | | ENDKLIREVKVITLKSKLVSDFRKDFQFYKVREINNYHHAHDAYLNAVVGTALI |
| | | | | KKYPKLESEFVYGDYKVYDVRKMIAKSEQEIGKATAKYFFYSNIMNFFKTEITL |
| | | | | ANGEIRKRPLIETNGETGEIVWDKGRDFATVRKVLSMPQVNIVKKTEVQTGGF |
| | | | | SKESIRPKRNSDKLIARKKDWDPKKYGGFLWPTVAYSVLVVAKVEKGKSKKL |
| | | | | KSVKELLGITIMERSSFEKNPIDFLEAKGYKEVKKDLIIKLPKYSLFELENGRKR |
| | | | | MLASAKQLQKGNELALPSKYVNFLYLASHYEKLKGSPEDNEQKQLFVEQHKH |
| | | | | YLDEIIEQISEFSKRVILADANLDKVLSAYNKHRDKPIREQAENIIHLFTLTRLG |
| | | | | APRAFKYFDTTIDPKQYRSTKEVLDATLIHQSITGLYETRIDLSQLGGDSGGSKR |
| | | | | TADGSEFEPKKKRKVGSGATNFSLLKQAGDVEENPGPMVSKGEEDNMAIIKE |
| | | | | FMRFKVHMEGSVNGHEFEIEGEGEGRPYEGTQTAKLKVTKGGPLPFAWDIL |
| | | | | SPQFMYGSKAYVKHPADIPDYLKLSFPEGFKWERVMNFEDGGVVTVTQDSS |
| | | | | LQDGEFIYKVKLRGTNFPSDGPVMQKKTMGWEASSERMYPEDGALKGEIKQ |
| | | | | RLKLKDGGHYDAEVKTTYKAKKPVQLPGAYNVNIKLDITSHNEDYTIVEQYER |
| | | | | AEGRHSTGGMDELYKSGGSPKKKRKV (SEQ ID NO: 14) |
| | nCas9- | HiFi- | QTC399 | MKRTADGSEFESPKKKRKVDKKYSIGLAIGTNSVGWAVITDEYKVPSKKFKVL |
| | HiFi | nCas9(D10A)- | | GNTDRHSIKKNLIGALLFDSGETAEATRLKRTARRRYTRRKNRICYLQEIFSNE |
| | | NG_P2A- | | MAKVDDSFFHRLEESFLVEEDKKHERHPIFGNIVDEVAYHEKYPTIYHLRKKL |
| | | mCherry | | VDSTDKADLRLIYLALAHMIKFRGHFLIEGDLNPDNSDVDKLFIQLVQTYNQLF |
| | | | | EENPINASGVDAKAILSARLSKSRRLENLIAQLPGEKKNGLFGNLIALSLGLTP |
| | | | | NFKSNFDLAEDAKLQLSKDTYDDDLDNLLAQIGDQYADLFLAAKNLSDAILLSD |
| | | | | ILRVNTEITKAPLSASMIKRYDEHHQDLTLLKALVRQQLPEKYKEIFFDQSKNG |
| | | | | YAGYIDGGASQEEFYKFIKPILEKMDGTEELLVKLNREDLLRKQRTFDNGSIPH |
| | | | | QIHLGELHAILRRQEDFYPFLKDNREKIEKILTFRIPYYVGPLARGNSRFAWMT |
| | | | | RKSEETITPWNFEEVVDKGASAQSFIERMTNFDKNLPNEKVLPKHSLLYEYFT |
| | | | | VYNELTKVKYVTEGMRKPAFLSGEQKKAIVDLLFKTNRKVTVKQLKEDYFKKI |
| | | | | ECFDSVEISGVEDRFNASLGTYHDLLKIIKDKDFLDNEENEDILEDIVLTLTLFE |
| | | | | DREMIEERLKTYAHLFDDKVMKQLKRRRYTGWGRLSRKLINGIRDKQSGKTIL |
| | | | | DFLKSDGFANANFMQLIHDDSLTFKEDIQKAQVSGQGDSLHEHIANLAGSPAI |
| | | | | KKGILQTVKVVDELVKVMGRHKPENIVIEMARENQTTQKGQKNSRERMKRIE |
| | | | | EGIKELGSQILKEHPVENTQLQNEKLYLYYLQNGRDMYVDQELDINRLSDYDV |
| | | | | DHIVPQSFLKDDSIDNKVLTRSDKNRGKSDNVPSEEVVKKMKNYWRQLLNAK |
| | | | | LITQRKFDNLTKAERGGLSELDKAGFIKRQLVETRQITKHVAQILDSRMNTKYD |
| | | | | ENDKLIREVKVITLKSKLVSDFRKDFQFYKVREINNYHHAHDAYLNAVVGTALI |
| | | | | KKYPKLESEFVYGDYKVYDVRKMIAKSEQEIGKATAKYFFYSNIMNFFKTEITL |
| | | | | ANGEIRKRPLIETNGETGEIVWDKGRDFATVRKVLSMPQVNIVKKTEVQTGGF |
| | | | | SKESIRPKRNSDKLIARKKDWDPKKYGGFVSPTVAYSVLVVAKVEKGKSKKL |
| | | | | KSVKELLGITIMERSSFEKNPIDFLEAKGYKEVKKDLIIKLPKYSLFELENGRKR |
| | | | | MLASARFLQKGNELALPSKYVNFLYLASHYEKLKGSPEDNEQKQLFVEQHKH |
| | | | | YLDEIIEQISEFSKRVILADANLDKYLSAYNKHRDKPIREQAENIIHLFTLTNLG |
| | | | | APRAFKYFDTTIDRKVYRSTKEVLDATLIHQSITGLYETRIDLSQLGGDSGGSKR |
| | | | | TADGSEFEPKKKRKVGSGATNFSLLKQAGDVEENPGPMVSKGEEDNMAIIKE |
| | | | | FMRFKVHMEGSVNGHEFEIEGEGEGRPYEGTQTAKLKVTKGGPLPFAWDIL |
| | | | | SPQFMYGSKAYVKHPADIPDYLKLSFPEGFKWERVMNFEDGGVVTVTQDSS |
| | | | | LQDGEFIYKVKLRGTNFPSDGPVMQKKTMGWEASSERMYPEDGALKGEIKQ |
| | | | | RLKLKDGGHYDAEVKTTYKAKKPVQLPGAYNVNIKLDITSHNEDYTIVEQYER |
| | | | | AEGRHSTGGMDELYKSGGSPKKKRKY (SEQ ID NO: 15) |
| CP- | apt-ABE8e | Com_93- | QTC126 | MKRTADGSEFESPKKKRKVKSIRCKNQNKLLFKADSFDHIEIRCPRCKRHIIML |
| deam- | | aa_TadA8e_ | | NACEHPTEKHCGKREKITHSDETVRYGGGGTGGGGSAEYVRALFDFNGNDE |
| inases: | | P2A-EGFP | | EDLPFKKGDILRIRDKPEEQWWNAEDSEGKRGMILVPYVEKYSGDYKDHDG |
| | | | | DYKDHDIDYKDDDDKSGSEVEFSHEYWMRHALTLAKRARDEREVPVGAVLV |
| | | | | LNNRVIGEGWNRAIGLHDPTAHAEIMALRQGGLVMQNYRLIDATLYVTFEPCV |
| | | | | MCAGAMIHSRIGRVVFGVRNSKRGAAGSLMNVLNYPGMNHRVEITEGILADE |
| | | | | CAALLCDFYRMPRQVFNAQKKAQSSINSKRTADGSEFEPKKKRKVGSGATN |
| | | | | FSLLKQAGDVEENPGPMVSKGEELFTGVVPILVELDGDVNGHKFSVSGEGE |
| | | | | GDATYGKLTLKFICTTGKLPVPWPTLVTTLTYGVQCFSRYPDHMKQHDFFKS |
| | | | | AMPEGYVQERTIFFKDDGNYKTRAEVKFEGDTLVNRIELKGIDFKEDGNILGH |
| | | | | KLEYNYNSHNVYIMADKQKNGIKYNFKIRHNIEDGSVQLADHYQQNTPIGDGP |
| | | | | VLLPDNHYLSTQSALSKDPNEKRDHMVLLEFVTAAGITLGMDELYKSGGSPK |
| | | | | KKRKV (SEQ ID NO: 16) |
| | apt- | TadA8.20_32- | QTC135 | MKRTADGSEFESPKKKRKVSSEVEFSHEYWMRHALTLAKRARDEREVPVGA |
| | ABE8.20 | aa_Com | | VLVLNNRVIGEGWNRAIGLHDPTAHAEIMALRQGGLVMQNYRLYDATLYSTF |
| | | | | EPCVMCAGAMIHSRIGRVVFGVRNAKTGAAGSLMDVLHHPGMNHRVEITEGI |
| | | | | LADEGAALLCRFFRMPRRVFNAQKKAQSSTDSGGSSGGSSGSETPGTSESA |
| | | | | TPESSGGSSGGSKSIRCKNCNKLLFKADSFDHIEIRCPRCKRHIIMLNACEHPT |
| | | | | EKHCGKREKITHSDETVRYGSKRTADGSEFEPKKKRKV (SEQ ID NO: 17) |
| | apt-CBE- | MCP_32- | QTC161 | MASNFTQFVLVDNGGTGDVTVAPSNFANGVAEWISSNSRSQAYKVTCSVRQ |
| | 3′end | aa_evo- | | SSAQKRKYTIKVEVPKVATQTVGGVELPVAAWRSYLNMELTIPIFATNSDCELI |
| | | rA1_32- | | VKAMQGLLKDGNPIPSAIAANSGIYSSGGSSGGSSGSETPGTSESATPESSG |
| | | aa_2xUGI_ | | GSSGGSSSKTGPVAVDPTLRRRIEPHEFEVFFDPRELRKETCLLYEINWGGR |
| | | P2A-EGFP | | HSIWRHTSQNTNKHVEVNFIEKFTTERYFCPNTRCSITWFLSWSPCGECSRAI |
| | | | | TEFLSRYPNVTLFIYIARLYHLANPRNRQGLRDLISSGVTIQIMTEQESGYCWH |
| | | | | NFVNYSPSNESHWPRYPHLWVRLYVLELYCIILGLPPCLNILRRKQSQLTSFTI |
| | | | | ALQSCHYQRLPPHILWATGLKSGGSSGGSSGSETPGTSESATPESSGGSSG |
| | | | | GSTNLSDIIEKETGKQLVIQESILMLPEEVEEVIGNKPESDILVHTAYDESTDEN |
| | | | | VMLLTSDAPEYKPWALVIQDSNGENKIKMLSGGSGGSGGSTNLSDIIEKETGK |
| | | | | QLVIQESILMLPEEVEEVIGNKPESDILVHTAYDESTDENVMLLTSDAPEYKPW |
| | | | | ALVIQDSNGENKIKMLSGGSKRTADGSEFEPKKKRKVGSGATNFSLLKQAGD |
| | | | | VEENPGPMVSKGEELFTGVVPILVELDGDVNGHKFSVSGEGEGDATYGKLTP |
| | | | | KFICTTGKLPVPWPTLVTTLTYGVQCFSRYPDHMKQHDFFKSAMPEGYVQE |
| | | | | RTIFFKDDGNYKTRAEVKFEGDTLVNRIELKGIDFKEDGNILGHKLEYNYNSHN |
| | | | | VYIMADKQKNGIKVNFKIRHNIEDGSVQLADHYQQNTPIGDGPVLLPDNHYLS |
| | | | | TQSALSKDPNEKRDHMVLLEFVTAAGITLGMDELYKSGGSPKKKRKV (SEQ |
| | | | | ID NO: 18) |
| | apt-CBE- | MCP_16- | QTC162 | MASNFTQFVLVDNGGTGDVTVAPSNFANGVAEWISSNSRSQAYKVTCSVRQ |
| | SL3 | aa_evo- | | SSAQKRKYTIKVEVPKVATQTVGGVELPVAAWRSYLNMELTIPIFATNSDCELI |
| | | rA1_32- | | VKAMQGLLKDGNPIPSAIAANSGIYSSGGSSGGSSGGSSGGSSSKTGPVAVD |
| | | aa_2xUGI_ | | PTLRRRIEPHEFEVFFDPRELRKETCLLYEINWGGRHSIWRHTSQNTNKHVE |
| | | P2A-EGFP | | VNFIEKFTTERYFCPNTRCSITWFLSWSPCGECSRAITEFLSRYPNVTLFIYIAR |
| | | | | LYHLANPRNRQGLRDLISSGVTIQIMTEQESGYCWHNFVNYSPSNESHWPRY |
| | | | | PHLWVRLYVLELYCIILGLPPCLNILRRKQSQLTSFTIALQSCHYQRLPPHILWA |
| | | | | TGLKSGGSGGSGGSTNLSDIIEKETGKQLVIQESILMLPEEVEEVIGNKPESDI |
| | | | | LVHTAYDESTDENVMLLTSDAPEYKPWALVIQDSNGENKIKMLSGGSGGSG |
| | | | | GSTNLSDIIEKETGKQLVIQESILMLPEEVEEVIGNKPESDILVHTAYDESTDEN |
| | | | | VMLLTSDAPEYKPWALVIQDSNGENKIKMLSGGSKRTADGSEFEPKKKRKVG |
| | | | | SGATNFSLLKQAGDVEENPGPMVSKGEELFTGVVPILVELDGDVNGHKFSVS |
| | | | | GEGEGDATYGKLTPKFICTTGKLPVPWPTLVTTLTYGVQCFSRYPDHMKQHD |
| | | | | FFKSAMPEGYVQERTIFFKDDGNYKTRAEVKFEGDTLVNRIELKGIDFKEDGN |
| | | | | ILGHKLEYNYNSHNVYIMADKQKNGIKVNFKIRHNIEDGSVQLADHYQQNTPI |
| | | | | GDGPALLPDNHYLSTQSALSKDPNEKRDHMVLLEFVTAAGITLGMDELYKSG |
| | | | | GSPKKKRKV (SEQ ID NO: 19) |
| Tandem | MOBE1 | MCP_32- | QTC281 | MASNFTQFVLVDNGGTGDVTVAPSNFANGVAEWISSNSRSQAYKVTCSVRQ |
| CP- | | aa_evo- | | SSAQKRKYTIKVEVPKVATQTVGGVELPVAAWRSYLNMELTIPIFATNSDCELI |
| deam- | | rA1_32- | | VKAMQGLLKDGNPIPSAIAANSGIYSSGGSSGGSSGSETPGTSESATPESSG |
| inases: | | aa_2xUGI_ | | GSSGGSSSKTGPVAVDPTLRRRIEPHEFEVFFDPRELRKETCLLYEINWGGR |
| | | PT2A_Com_93- | | HSIWRHTSQNTNKHVEVNFIEKFTTERYFCPNTRCSITWFLSWSPCGECSRAI |
| | | aa_TadA8e | | TEFLSRYPNYTLFIYIARLHHLANPRNRQGLRDLISSGVTIQIMTEQESGYCWH |
| | | | | NFVNYSPSNESHWPRYPHLWVRLYVLELYCIILGLPPCLNILRRKQSQLTSFTI |
| | | | | ALQSCHYQRLPPHILWATGLKSGGSSGGSSGSETPGTSESATPESSGGSSG |
| | | | | GSTNLSDIIEKETGKQLVIQESILMLPEEVEEVIGNKPESDILVHTAYDESTDEN |
| | | | | VMLLTSDAPEYKPWALVIQDSNGENKIKMLSGGSGGSGGSTNLSDIIEKETGK |
| | | | | QLVIQESILMLPEEVEEVIGNKPESDILVHTAYDESTDENVMLLTSDAPEYKPW |
| | | | | ALVIQDSNGENKIKMLSGGSKRTADGSEFEPKKKRKVGSGATNFSLLKQAGD |
| | | | | VEENPGPGSGEGRGSLLTCGDVEENPGPMKRTADGSEFESPKKKRKVKSIR |
| | | | | CKNCNKLLFKADSFDHIEIRCPRCKRHIIMLNACEHPTEKHCGKREKITHSDET |
| | | | | VRYGGGGTGGGGSAEYVRALFDFNGNDEEDLPFKKGDILRIRDKPEEQWW |
| | | | | NAEDSEGKRGMILVPYVEKYSGDYKDHDGDYKDHDIDYKDDDDKSGSEVEF |
| | | | | SHEYWMRHALTLAKRARDEREVPVGAVLVLNNRVIGEGWNRAIGLHDPTAH |
| | | | | AEIMALRQGGLVMQNYRLIDATLYYTFEPCVMCAGAMIHSRIGRVVFGVRNS |
| | | | | KRGAAGSLMNVLNYPGMNHRVEITEGILADECAALLCDFYRMPRQVFNAQKK |
| | | | | AQSSINSKRTADGSEFEPKKKRKY (SEQ ID NO: 20) |
| | MOBE2 | MCP_32- | QTC282 | MASNFTQFVLVDNGGTGDVTVAPSNFANGVAEWISSNSRSQAYKVTCSVRQ |
| | | aa_evo- | | SSAQKRKYTIKVEVPKVATQTVGGVELPVAAWRSYLNMELTIPIFATNSDCELI |
| | | rA1_32- | | VKAMQGLLKDGNPIPSAIAANSGIYSSGGSSGGSSGSETPGTSESATPESSG |
| | | aa_2xUGI_ | | GSSGGSSSKTGPVAVDPTLRRRIEPHEFEVFFDPRELRKETCLLYEINWGGR |
| | | PT2A_ | | HSIWRHTSQNTNKHVEVNFIEKFTTERYFCPNTRCSITWFLSWSPCGECSRAI |
| | | TadA8.20_32- | | TEFLSRYPNYTLFIYIARLYHLANPRNRQGLRDLISSGVTIQIMTEQESGYCWH |
| | | aa_Com | | NFVNYSPSNESHWPRYPHLWVRLYVLELYCIILGLPPCLNILRRKQSQLTSFTI |
| | | | | ALQSCHYQRLPPHILWATGLKSGGSSGGSSGSETPGTSESATPESSGGSSG |
| | | | | GSTNLSDIIEKETGKQLVIQESILMLPEEVEEVIGNKPESDILVHTAYDESTDEN |
| | | | | VMLLTSDAPEYKPWALVIQDSNGENKIKMLSGGSGGSGGSTNLSDIIEKETGK |
| | | | | QLVIQESILMLPEEVEEVIGNKPESDILVHTAYDESTDENVMLLTSDAPEYKPW |
| | | | | ALVIQDSNGENKIKMLSGGSKRTADGSEFEPKKKRKVGSGATNFSLLKQAGD |
| | | | | VEENPGPGSGEGRGSLLTCGDVEENPGPMKRTADGSEFESPKKKRKVSSE |
| | | | | VEFSHEYWMRHALTLAKRARDEREVPVGAVLVLNNRVIGEGWNRAIGLHDP |
| | | | | TAHAEIMALRQGGLVMQNYRLYDATLYSTFEPCVMCAGAMIHSRIGRVVFGV |
| | | | | RNAKTGAAGSLMDVLHHPGMNHRVEITEGILADECAALLCRFFRMPRRVFNA |
| | | | | QKKAQSSTDSGGSSGGSSGSETPGTSESATPESSGGSSGGSKSIRCKNQNK |
| | | | | LLFKADSFQHIEIRCPRCKRHIIMLNACEHPTEKHCGKREKITHSDETVRYGSK |
| | | | | RTADGSEFEPKKKRKV (SEQ ID NO: 21) |
| | MOBE3 | MCP_16- | QTC283 | MASNFTQFVLVDNGGTGDVTVAPSNFANGVAEWISSNSRSQAYKVTCSVRQ |
| | | aa_evo- | | SSAQKRKYTIKVEVPKVATQTVGGVELPVAAWRSYLNMELTIPIFATNSDCELI |
| | | rA1_32- | | VKAMQGLLKDGNPIPSAIAANSGIYSSGGSSGGSSGGSSGGSSSKTGPVAVD |
| | | aa_2xUGI_ | | PTLRRRIEPHEFEVFFDPRELRKETCLLYEINWGGRHSIWRHTSQNTNKHVE |
| | | PT2A_Com_93- | | VNFIEKFTTERYFCPNTRCSITWFLSWSPCGECSRAITEFLSRYPNVTLFIYIAR |
| | | aa_TadA8e | | LYHLANPRNRQGLRDLISSGVTIQIMTEQESGYCWHNFVNYSPSNESHWPRY |
| | | | | PHLWVRLYVLELYCIILGLPPCLNILRRKQSQLTSFTIALQSCHYQRLPPHILWA |
| | | | | TGLKSGGSGGSGGSTNLSDIIEKETGKQLVIQESILMLPEEVEEVIGNKPESDI |
| | | | | LVHTAYDESTDENVMLLTSDAPEYKPWALVIQDSNGENKIKMLSGGSGGSG |
| | | | | GSTNLSDIIEKETGKQLVIQESILMLPEEVEEVIGNKPESDILVHTAYDESTDEN |
| | | | | VMLLTSDAPEYKPWALVIQDSNGENKIKMLSGGSKRTADGSEFEPKKKRKVG |
| | | | | SGATNFSLLKQAGDVEENPGPGSGEGRGSLLTCGDVEENPGPMKRTADGS |
| | | | | EFESPKKKRKVKSIRCKNCNKLLFKADSFDHIEIRCPRCKRHIIMLNACEHPTE |
| | | | | KHCGKREKITHSDETVRYGGGGTGGGGSAEYVRALFDFNGNDEEDLPFKKG |
| | | | | DILRIRDKPEEQWWNAEDSEGKRGMILVPYVEKYSGDYKDHDGDYKDHDIDY |
| | | | | KDDDDKSGSEVEFSHEYWMRHALTLAKRARDEREVPVGAVLVLNNRVIGEG |
| | | | | WNRAIGLHDPTAHAEIMALRQGGLVMQNYRLIDATLYVTFEPCVMCAGAMIH |
| | | | | SRIGRVVFGYRNSKRGAAGSLMNYLNYPGMNHRVEITEGILADECAALLCDF |
| | | | | YRMPRQVFNAQKKAQSSINSKRTADGSEFEPKKKRKV (SEQ ID NO: 22) |
| | MOBE4 | MCP_16- | QTC284 | MASNFTQFVLVDNGGTGDVTVAPSNFANGVAEWISSNSRSQAYKVTCSVRQ |
| | | aa_evo- | | SSAQKRKYTIKVEVPKVATQTVGGVELPVAAWRSYLNMELTIPIFATNSDCELI |
| | | rA1_32- | | VKAMQGLLKDGNPIPSAIAANSGIYSSGGSSGGSSGGSSGGSSSKTGPVAVD |
| | | aa_2xUGI_ | | PTLRRRIEPHEFEVFFDPRELRKETCLLYEINWGGRHSIWRHTSQNTNKHVE |
| | | PT2A_ | | VNFIEKFTTERYFCPNTRCSITWFLSWSPCGECSRAITEFLSRYPNVTLFIYIAR |
| | | TadA8.20_32- | | LYHLANPRNRQGLRDLISSGVTIQIMTEQESGYCWHNFVNYSPSNESHWPRY |
| | | aa_Com | | PHLWVRLYVLELYCIILGLPPCLNILRRKQSQLTSFTIALQSCHYQRLPPHILWA |
| | | | | TGLKSGGSGGSGGSTNLSDIIEKETGKQLVIQESILMLPEEVEEVIGNKPESDI |
| | | | | LVHTAYDESTDENVMLLTSDAPEYKPWALVIQDSNGENKIKMLSGGSGGSG |
| | | | | GSTNLSDIIEKETGKQLVIQESILMLPEEVEEVIGNKPESDILVHTAYDESTDEN |
| | | | | VMLLTSDAPEYKPWALVIQDSNGENKIKMLSGGSKRTADGSEFEPKKKRKVG |
| | | | | SGATNFSLLKQAGDVEENPGPGSGEGRGSLLTCGDVEENPGPMKRTADGS |
| | | | | EFESPKKKRKVSSEVEFSHEYWMRHALTLAKRARDEREVPVGAVLVLNNRVI |
| | | | | GEGWNRAIGLHDPTAHAEIMALRQGGLVMQNYRLYDATLYSTFEPCVMCAG |
| | | | | AMIHSRIGRVVFGVRNAKTGAAGSLMDVLHHPGMINHRVEITEGILADECAALL |
| | | | | CRFFRMPRRVFNAQKKAQSSTDSGGSSGGSSGSETPGTSESATPESSGGS |
| | | | | SGGSKSIRCKNCNKLLFKADSFDHIEIRCPRCKRHIIMLNACEHPTEKHCGKR |
| | | | | EKITHSDETVRYGSKRTADGSEFEPKKKRKV (SEQ ID NO: 23) |
| Fluor- | MOBE1-2 | GFP(A110V/ | QTC311 | MVSKGEELFTGVVPILVELDGDVNGHKFSVSGEGEGDATYGKLTLKFICTTGK |
| escent | reporter | L202S); A110V | | LPVPWPTLVTTLTYGVQCFSRYPDHMKQHDFFKSAMPEGYVQERTIFFKDD |
| reporter | | gRNA + com- | | GNYKTRVEVKFEGDTLVNRIELKGIDFKEDGNILGHKLEYNYNSHNVYIMADK |
| | | 3′end; L202S | | QKNGIKVNFKIRHNIEDGSVQLADHYQQNTPIGDGPVLLPDNHYSSTQSALSK |
| | | gRNA + MS2- | | DPNEKRDHMVLLEFVTAAGITLGMDELYK (SEQ ID NO: 24) |
| | | 3′end | | |
| plasmids: | MOBE3-4 | GFP(A110V/ | QTC312 | MVSKGEELFTGVVPILVELDGDVNGHKFSVSGEGEGDATYGKLTLKFICTTGK |
| | reporter | L202S); A110V | | LPVPWPTLVTTLTYGVQCFSRYPDHMKQHDFFKSAMPEGYVQERTIFFKDD |
| | | gRNA + com- | | GNYKTRVEVKFEGDTLVNRIELKGIDFKEDGNILGHKLEYNYNSHNVYIMADK |
| | | 3′end; L202S | | QKNGIKVNFKIRHNIEDGSVQLADHYQQNTPIGDGPVLLPDNHYSSTQSALSK |
| | | gRNA + MS2- | | DPNEKRDHMVLLEFVTAAGITLGMDELYK (SEQ ID NO: 24) |
| | | SL3 | | |

[symbol]

indicates data missing or illegible when filed

In certain embodiments, a simple fluorescence-based strategy was also developed which allows for enrichment of cells with co-occurring orthogonal edits, which enabled up to a 35-fold increase in editing efficiency. With this enrichment strategy, it enabled up to 25% of cells to have co-occurring orthogonal edits, with only 1.1% of cells having undesired, off-target edits.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited.

All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Reference to “a/an” chemical compound, therapeutic agent, and pharmaceutical composition each refers to one or more molecules of the chemical compound, therapeutic agent, and pharmaceutical composition rather than being limited to a chemical compound, therapeutic agent, and pharmaceutical composition, the one or more molecules may or may not be identical, so long as they fall under the category of the chemical compound, therapeutic agent, and pharmaceutical composition. Thus, for example, “a” therapeutic agent is interpreted to include one or more molecules of the therapeutic agent, where the therapeutic agent molecules may or may not be identical (e.g., comprising different isotope abundances and/or different degrees of hydration or in equilibrium with different conjugate base or conjugate acid forms).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of inflammation associated with any disease in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts.

In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

It is understood that unless otherwise specified, temperatures referred to herein are based on atmospheric pressure (i.e., one atmosphere).

Now having described the aspects of the present disclosure, in general, the following provides details of the present disclosure. While the present disclosure is described in connection with the following details and the corresponding text and figures, there is no intent to limit the present disclosure to the descriptions. On the contrary, the intent is to cover all alternatives, modifications, and equivalents included within the spirit and scope of the present disclosure.

The following descriptions are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The present disclosure provides the genome editing system using wild-type CRISPR/Cas9 or CRISPR/Cas12 enzymes and the limitations of the existing methods to perform multiplexed editing were compared as to the genome editing system disclosed herein. The existing genome editing tools function by cutting the DNA backbone as the first step to installing mutations (they use double-strand DNA breaks, or DSBs). These tools inherently suffer from low precision, as random insertion/deletion (indel) products get introduced at the cut site, in addition to the desired point mutation. Overall, the efficiency of the process is quite low as well. The low precision and efficiency of DSB-reliant technologies is exacerbated, when attempting to multiplex point mutation introduction, as success rates decrease exponentially with the number of desired edits.

Introducing two or more DSBs throughout the genome results in translocations (when multiplexing at distinct chromosomes), large-scale deletions (when multiplexing within the same chromosome), chromosomal aberrations, and/or p53-mediated apoptosis. Because the system described herein uses alternative intermediates (e.g., uracils and inosines, instead of DSBs, these issues do not apply to the system of the present disclosure.

Using current CBE and ABE technologies: The way current base editors work is that the deaminase enzymes (which do the nucleobase chemistry) are directly fused to the Cas9 enzyme. A piece of RNA (called the gRNA) is then programmed to direct the Cas9 enzyme to particular genomic loci. In the system of the present disclosure, the deaminases are tethered to the gRNA instead of the Cas9 enzyme via a coat protein-aptamer interaction.

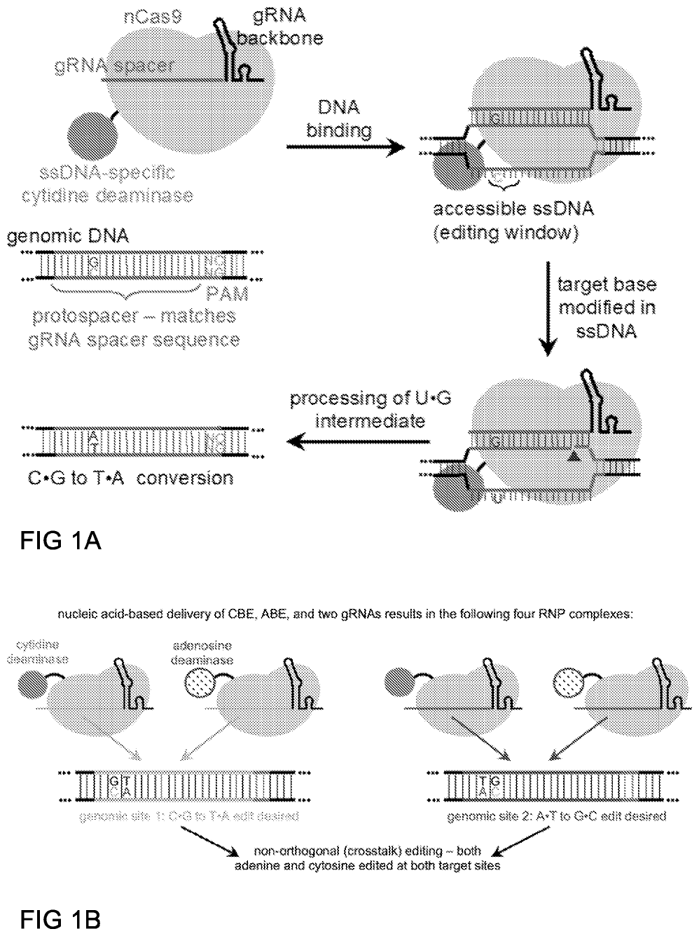

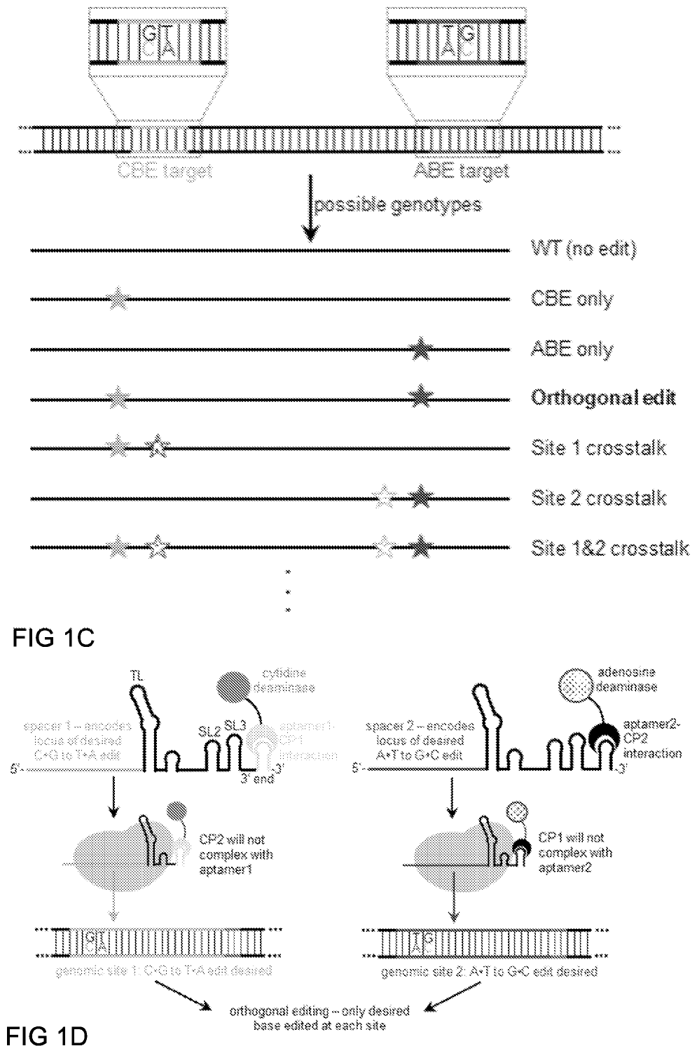

The lack of precise genome editing tools to orthogonally multiplex SNV introduction has hindered the investigation and treatment of co-occurring variants. BEs are ideal genome editing tools to enable multiplexed point mutation introduction, as they introduce SNVs with high efficiency and precision, and their intermediates are less toxic than DSBs. However, current BEs can only be multiplexed when using a single BE variant to perform editing at various loci. When multiplexing two or more BE variants, gRNA crosstalk occurs, resulting in all BEs performing editing at all loci (FIGS. 1B-1C). This can be prevented if the BE:gRNA complexes are delivered as purified ribonucleoprotein (RNP) complexes, but a lack of commercial sources that produce high-quality BE protein makes this strategy inaccessible to most laboratories. Furthermore, BE variants that utilize orthogonal Cas proteins (such as SpCas9, SaCas9, and Cas12 variants) could be cleanly multiplexed. However, the high efficiency and significantly less restrictive PAM requirements of the SpCas9-derived BEs cause this alternative to suffer from a variety of shortcomings.

The genome editing approach disclosed herein utilized RNA aptamer-coat protein technologies to develop and characterize four multiplexed orthogonal base editor (MOBE) systems that enable the introduction of combinations of C⋅G to T⋅A and A⋅T to G⋅C point mutations simultaneously throughout the genome with high efficiency and precision. In certain embodiments, a reporter plasmid was also generated that allows for the enrichment of cells with orthogonal multiplexed edits and enabled an X-fold increase in editing efficiency. This simple enrichment strategy can aid researchers in generating cell-based models of SNV combinations relevant to genetic disease.

In certain embodiments, aach MOBE is comprised of a unique combination of an aptamer-based CBE system and an aptamer-based ABE system. To develop each aptamer-based BE system, a variety of constructs were screened through in which one of four different aptamers was imbedded within the TL, SL2, SL3, or 3′-end of the gRNA. These aptamer-gRNA constructs were then combined with corresponding coat protein-deaminase fusions, in which the architectures (N- and C-terminal fusions), deaminases, and linkers were varied. It was found that for the ABE aptamer systems, the use of eighth-generation evolved TadA deaminases was the single most important factor to facilitate consistently high editing activity across all tested sites, followed by the aptamer-coat protein system (the com system) and the location of the aptamer (3′-end embedded). The CBE aptamer systems were generally less stringent in their design, but again the use of a highly evolved deaminase (the evoAPOBECi cytidine deaminase) provided the largest boost in editing efficiency out of all the modifications tested.

Out of the four systems, MOBE3 typically facilitates the highest editing efficiency without selection, while MOBE1 typically has the highest orthogonality scores. However, both of these metrics are site-dependent, and screening all four systems for a given protospacer combination is suggested. If low editing efficiencies are observed, the MOBE fluorescent reporter is recommended. Again, while MOBE2 typically facilitated the highest rates of co-occurring orthogonal edits, this was observed to be site-dependent.

The MOBE system can be easily modified to implement additional Sp-nCas9 variants. Specifically, the targeting scope can be expanded by using the near PAM-less SpRY SpCas9 variant, which will be especially useful with the relatively narrow editing window.¹⁵ Additionally, if specificity is a high priority, then high-fidelity nCas9 variants (such as HF1 or SuperFi)^{36,37} can be implemented to mitigate gRNA-dependent off-target effects. In addition to the fluorescent reporter, on-target editing activity may be improved through the addition of a protective pseudoknot to the 3′-end of the aptamer-gRNAs; this strategy was shown to increase prime editing efficiency by preventing endogenous exonucleases from degrading 3′ extensions on pegRNAs.³⁸ Truncated pegRNAs (due to degradation of the 3′ extension) can sequester prime editor protein and are still able to engage to the target site for nicking, but lack the ability to install the prime edit. This proposed mechanism of inhibition would also apply to the aptamer-BE systems, especially those with 3′-end aptamers. Degradation of the 3′-end could cause unproductive nCas9 activity at the target site without the ability to recruit a deaminase enzyme.

The MOBE systems disclosed herein are powerful tools for disease modelling. They can be used to analyze epistatic effects of modifier SNVs observed in individuals with altered disease penetrance or common variants found in the afflicted population(s). MOBEs can probe SNVs in non-coding regions such as cis-regulatory elements that may modulate expressivity of a coding variant. They can also be applied to exploratory studies of the contributions of VUS in polygenic diseases, such as cancer, diabetes, and schizophrenia. The MOBE systems are well-positioned to model or therapeutically correct digenic diseases, which are variant combinations that are known to cause a disease (examples include Long QT syndrome and Brugada syndrome).^{12,39} Currently, there are 421 digenic variant combinations reported in the oligogenic diseases database (OLIDA), and 114 can be modelled by multiplexing a CBE, ¹¹ can be modelled by multiplexing an ABE, and 54 would require a MOBE system to be modelled.⁴⁰

The MOBE systems can be leveraged for complex genome engineering with other effector modalities that use the SpCas9 ortholog. Multiplexed orthogonal base editing could be compatible with aptamer-recruited CRISPRi, CRISPRa, or targeted integration.^{18,41} Possible applications include base editor screens to probe epistasis/synthetic lethal gene interactions, metabolic reprogramming, genetic logic circuits, and event recording.^{42,43}

Now having described the aspects of the present disclosure, in general, the following Examples describe some additional and/or more detailed aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Methods and Materials

General methods and cloning. All primers were ordered through Integrated DNA technologies (IDT). All PCR reactions were performed with Phusion DNA Green High-Fidelity Polymerase (F534L, Thermo Fisher) or Phusion U (F556L, Thermo Fisher) where appropriate. gRNA plasmids were cloned by site directed mutagenesis using a 5′ tail in the primer to replace the 20 nt spacer region, as described in Vasquez et al. Basic Protocol 1.44 Similarly, aptamers were inserted into the S. pyogenes gRNA backbone using primers with 5′ tails. Golden gate acceptor plasmids were also created for each of the final aptamer-gRNA fusions (MS2-SL3, MS2-3′end, and Com-3′end) that are available on Addgene. Codon optimized (GenScript) coat proteins were ordered as a gBlock gene fragment (IDT) and subcloned into CP-deaminase fusions with USER cloning. Linkers and deaminases were repurposed from established base editors ancBE4-max, ABE7.10-max, and target-AID (93-aa linker). Evolved base editor plasmids were obtained from Addgene: pBT281 (evo-APOBEC1-BE4), NG-ABE8e (Addgene plasmid #122611 and 138491), ABE8.20-m (Addgene plasmid #136300), and pCMV-BE4-RrA3F (Addgene plasmid #138340).^{29,30,32,45} The deaminase domains were subcloned into nCas9-NG backbones for parental BE controls and into CP-containing plasmids for aptamer-BE systems. The “all-in-one” reporter plasmid was cloned via USER cloning to insert the 2×-dead-GFP and aptamer-containing gRNAs. Unique golden gate sites were designed for each gRNA spacer (based on pDG461, Addgene plasmid #100902) and the final plasmid was assembled using the established protocol.⁵² Endotoxin-free plasmids were prepared with ZymoPURE II Midiprep Kit (D4201, Zymo Research).

General human tissue culture. HEK293T cells (CRL-3216, ATCC) were cultured at 37° C. with 5% CO2 in DMEM+GlutaMAX (10566-024, Thermo Fisher) supplemented with 10% (v/v) fetal bovine serum (10437-028, Thermo Fisher).

Transfections for base editing experiments. HEK293T cells were seeded at 50,000 cells/well in a 48-well plate and transfected after 16 hours at ˜70% confluency. DNA mixes were created in a total volume of 12.5 μL with Opti-MEM reduced-serum medium (31985-070, Thermo Fisher), and combined with 1.5 μL of Lipofectamine 2000 regent in 11 μL of Opti-MEM, according to the manufacturer's instructions. Cells were incubated for 72 hr, washed with 150 μL PBS, and lysed for genomic DNA with freshly prepared buffer containing 100 mM Tris-HCl (pH 7.0), 0.05% SDS, and 25 μg ml⁻¹ Proteinase K (P8107S, New England Biolabs). Samples were incubated on a thermocycler at 37° C. for 1 hr, 80° C. for 30 min, and then held at 4° C. For individual aptamer-BE experiments (optimization and characterization), 250 ng gRNA plasmid was combined with 850 ng of nCas9-NG and 650 ng CP-deaminase (or 1000 ng parental base editor). For multiplexed orthogonal base editing experiments, 500 ng of Cas9n-NG and 500 ng of MOBE (or 500 ng of each parental base editor) were combined with 125 ng of each gRNA plasmid. For fluorescent enrichment experiments, an additional 250 ng of reporter plasmid was added.

Next-generation sequencing. Samples were prepared for targeted amplicon sequencing as described in Vasquez et al. Alternative Protocol 1. Briefly, 1 μL of genomic DNA was added in a 25 μL PCR reaction with 0.2 μM primers (listed in Supplementary Data 1) for round 1 and amplified for 24-27 cycles (minimal amount to avoid PCR bias). After confirmation on a 2% agarose gel, round 2 PCR was performed to barcode samples with 8-12 cycles. Samples were pooled, purified by gel extraction, and then quantified by Qubit with the dsDNA HS assay kit (032854, Thermo Fisher). Sequencing was performed on an Illumina MiniSeq (2×151 paired end reads) per the manufacturer's instructions.

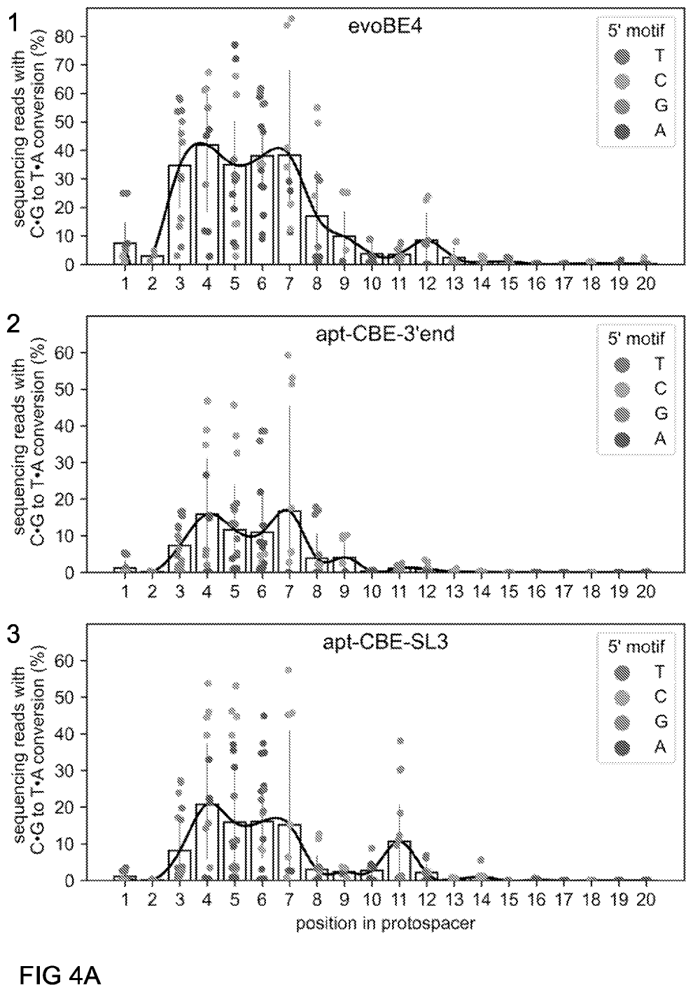

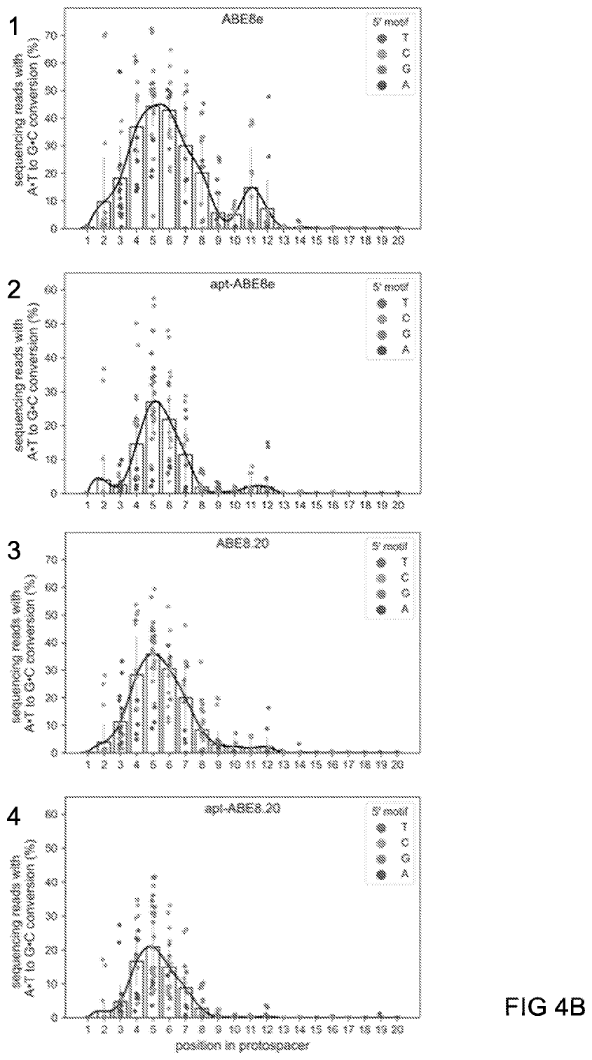

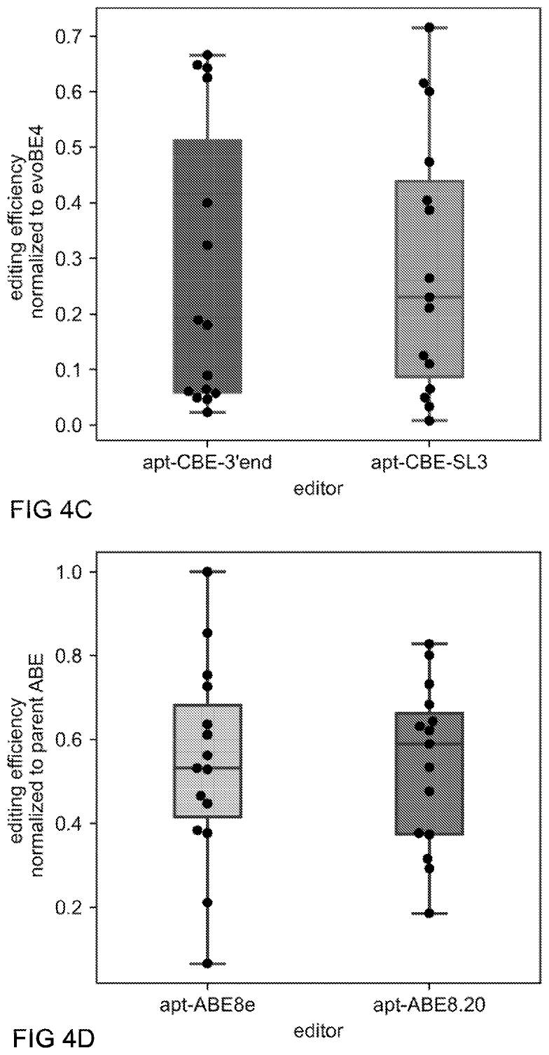

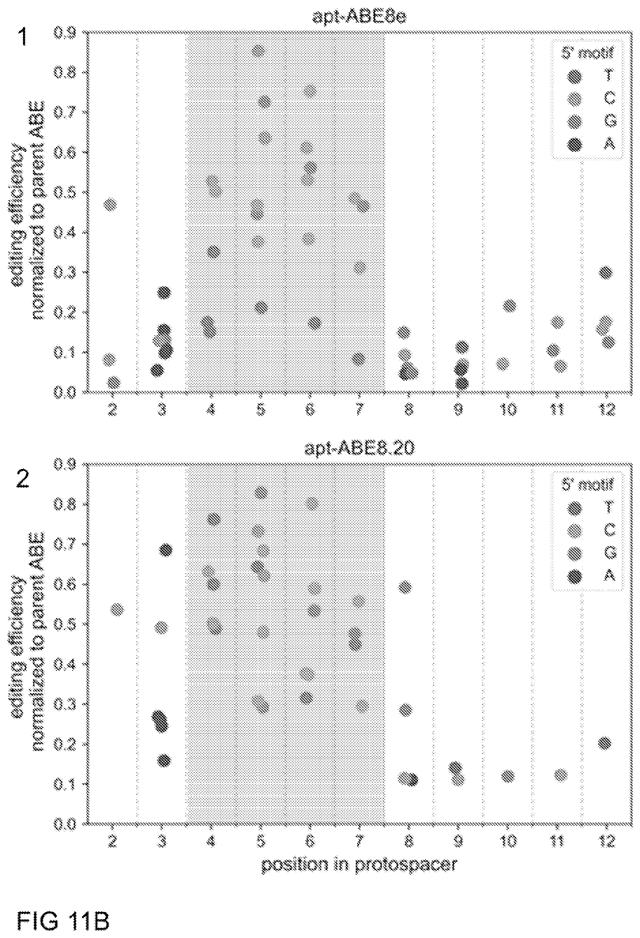

Data Analysis and Statistics. Next-generation sequencing data were demultiplexed and trimmed with Illumina Local Run Manager Generate FASTQ analysis module v2.0. The FASTQ files were analyzed with CRISPResso2 (version 2.0.20b) on batch mode (parameters: --base_edit -wc -10 -w 10 -q 30) to assess genomic base editing efficiencies.⁵³ All base editing efficiencies values are reported as a percent of total DNA sequencing reads with editing (from the CRISPResso2 nucleotide percent summary output) and as averages of independent biological replicates. Aptamer base editor characterization data (FIGS. 4A-4D and FIGS. 11A-11B) were analyzed on pandas (v1.4.2) and plotted on seaborn (v0.11.2). Fold-change values compared to different types of editors were computed as the average editing efficiency of test editor divided by the average editing efficiency of control editors ±propagation of uncertainty of the SEM. Orthogonality scores were calculated as the percent of total DNA sequencing reads with on-target conversion divided by the percent of total DNA sequencing reads with crosstalk of the same conversion (e.g. average C⋅G to T⋅A editing at the CBE target divided by average C⋅G to T⋅A editing at the ABE target) ±propagation of uncertainty of the SEM. Fold-change enrichment values for the MOBE fluorescent reporter represent average editing efficiencies of FACS sorted GFP+cells divided by average editing efficiencies of FACS sorted single cells regardless of color ±propagation of uncertainty of the standard deviation.

Co-occurring edit analysis. Sequencing data in fastq format were aligned to the Homo sapiens Hg38 reference genome using the Burrows-Wheeler Aligner.⁵⁴ A quality control filter was applied using SAMtools,⁵⁵ and only reads with quality scores greater than or equal to 2 were considered in the analysis. Each protospacer containing the desired on-target as well as potential off-target edits was mapped onto the respective paired-end reads. The Levenshtein edit distance between the protospacers (CBE and ABE), and the sequencing reads were calculated to quantify all insertions, deletions, and substitutions in the protospacer region of the aligned reads. In addition to the on-target and crosstalk edits, sequences that differed from the protospacer in more than three insertions, deletions, and/or substitutions were excluded from the analysis due to poor sequencing/alignment quality. The genotypes at each on-target edit site as well as potential crosstalk sites were extracted as a haplotype to quantify co-occurring editing efficiency. Haplotypes called from the paired-end sequencing reads were then categorized as 1) wild-type (no-edits), 2) orthogonal edit, 3) CBE on-target edit, 4) ABE on-target edit, 5) dual edits only, 6) crosstalk only, 7) CBE or ABE only, and 8) all other genotypes. This software is termed MOBEnto: Quantification of Haplotypes from Multiplexed Genome Editing Using Next-Generation Sequencing Data.

Fluorescence-activated Cell Sorting (FACS). Cells were washed with 150 μL of PBS and detached with 30 μL of Accumax (STEMCELL Technologies) at room temperature. After 1 min, cells were resuspended with 170 μL of cold PBS, passed through a 35 μm cell strainer into a test tube, and kept on ice. Samples were run on a S3e Cell Sorter (BioRad), using cells expressing either mCherry or GFP to set scatter and fluorescence gates. To enrich for base-edited cells, 5,000-50,000 GFP and mCherry double-positive single cells were collected per sample. For unenriched samples, 50,000 singlets were collected, regardless of color. For all samples, cells were sorted using the S3e machine's ProSort software (v1.6, BioRad) on “Enrich” mode directly into 1.5 mL tubes containing 500 μL of cold PBS. Cells were pelleted at 300×g for 10 min and the supernatant was gently pipetted off. Pellets were resuspended in lysis buffer (100 mM Tris-HCl [pH 7.0], 0.05% SDS, and 25 μg ml⁻¹ Proteinase K [P8107S, New England Biolabs]) at a final concentration of 2,000 cells/μL to be used as template for targeted amplicon sequencing.

Data availability. Next-generation sequencing data are available on the NCBI Sequencing Read Archive database under project number PRJNA836633. Plasmids from are available at Addgene.

### Example 2

### Design of Orthogonal Aptamer-Base Editor Systems

To engineer a system that enables orthogonal, multiplexed C⋅G to T⋅A and A⋅T to G⋅C point mutation introduction at distinct genomic loci using only the SpCas9 homolog, RNA aptamer technologies were utilized (FIG. 1D). Recruitment of the DNA modifier (the cytosine or adenosine deaminase) directly to its gRNA (which encodes the genomic locus to be edited) via an aptamer-binding protein interaction provides a modular system that avoids crosstalk. Furthermore, it theoretically allows for as many simultaneous orthogonal nucleotide conversions as there are orthogonal RNA aptamer-binding protein systems (currently, there are at least four well-characterized systems with more possible through directed evolution).¹⁸⁻²¹ Finally, RNA aptamer-mediated effector recruitment has been used successfully with Cas9 systems previously; this includes orthogonal fluorescent labelling,^{19,20} transcriptional reprogramming,^{18,22} lncRNA recruitment,²³ and random mutagenesis with a hyperactive cytosine deaminase.²⁴

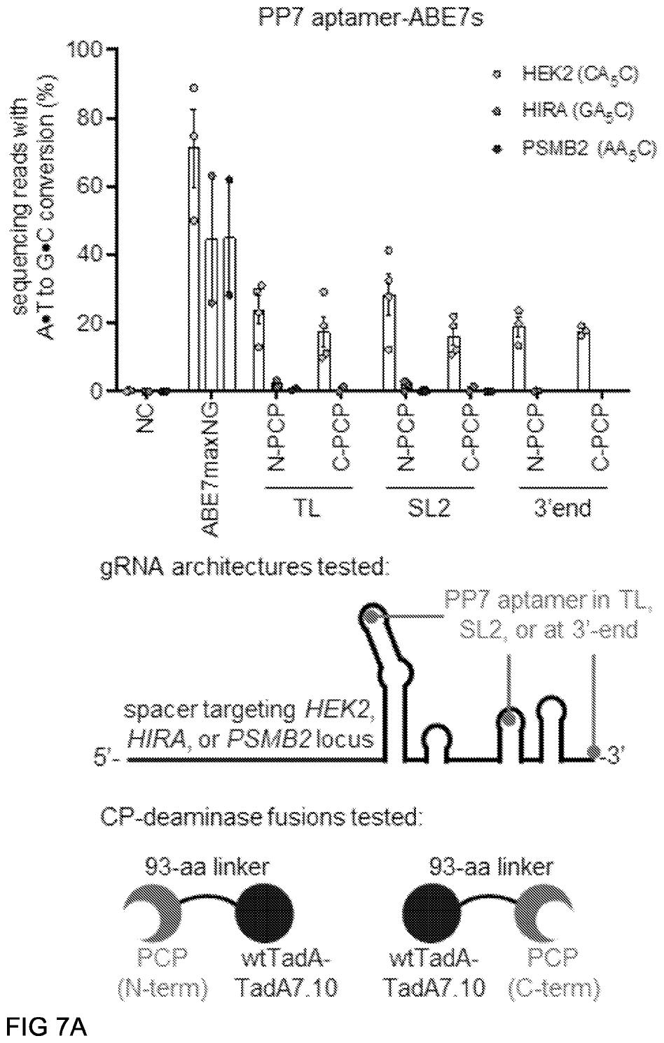

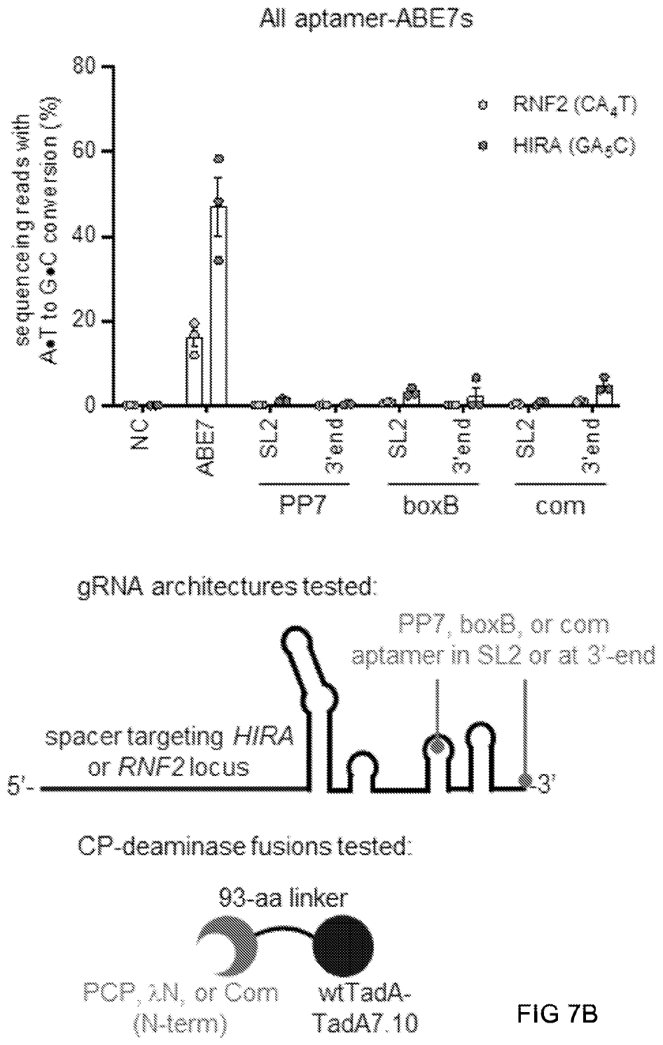

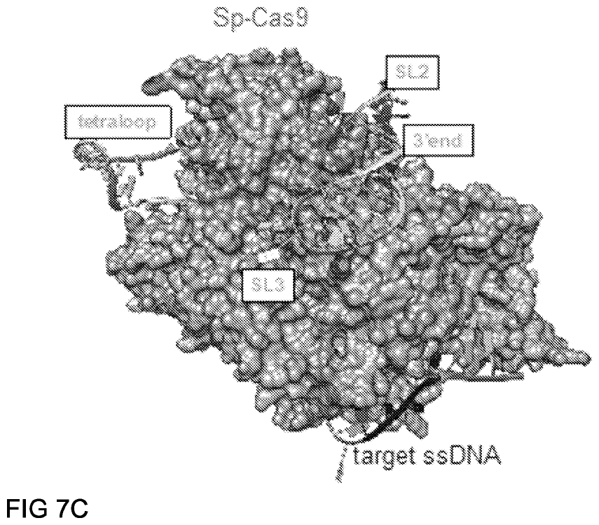

Structures of the Cas9:gRNA complex show that the tetraloop, stem-loop 2 (SL2), and 3′-end of the gRNA physically protrude from the complex and do not contact any Cas9 residues (FIG. 7C). As such, RNA sequence additions are tolerated in these regions without effecting gRNA stability, Cas9:gRNA complex formation, or Cas9:gRNA-DNA binding.²³ Therefore, orthogonal recruitment of each nucleobase-specific modifying enzyme to its desired target site can be enabled by embedding RNA aptamers within the gRNA at these locations, and fusing the complimentary coat protein (CP) to either the cytidine or adenosine ssDNA modifier (FIG. 1D). Initially, the most commonly used orthogonal aptamer systems: the MS2 bacteriophage system and the Pseudomonas phage PP7 system, were focused on. The MS2 coat protein (MCP) and PP7 coat protein (PCP) are small proteins (129- and 127-amino acids) that bind as dimers to their respective RNA stem-loops^{25,26} and do not interact with non-cognate binding proteins.^{18,19} As cytidine deaminase-derived editing systems have been previously developed using the MS2 aptamer,^{24,27} the engineering efforts were focused on the development of an ABE-aptamer system.

### Example 3

Initial Aptamer-ABE Constructs Introduce Point Mutations, but with Low, Variable Efficiencies

six ABE aptamer systems were generated in which PCP was fused to either the N- or C-terminus of wild type (wt)Tad-TadA7.10 (the heterodimeric seventh-generation deoxyadenosine deaminase construct)⁸ via a 93-amino acid (aa) flexible linker (FIG. 7A), and the PP7 aptamer embedded in the TL, SL2, or 3′-end of the Sp-gRNA (FIG. 7A). All combinations of gRNA-aptamer and wtTad-TadA7.10-PCP fusion were tested by assessing editing efficiencies at 3 well-characterized genomic sites (HEK2, HIRA, and PSMB2). In all experiments, HEK293T cells were transfected with plasmids encoding nCas9-NG (with bicistronic mCherry expression to allow for evaluation of transfection efficiency), wtTad-TadA7.10-PCP fusion, and gRNA-aptamer. Cells were then lysed after 72 hours, genomic loci of interest were amplified, and A⋅T to G⋅C editing efficiencies were quantified by next generation sequencing (NGS). Initial experiments showed that the ABE systems could install targeted point mutations, but with drastically lower editing efficiencies compared to the parental ABE7.10max-NG construct. Furthermore, base editing efficiencies by the aptamer complexes varied greatly by genomic site. Specifically, it was observed similar A⋅T to G⋅C editing efficiencies at the HEK2 locus by the six systems (average A⋅T to G⋅C editing of 20.5±2.0% by the six systems, see FIG. 7A and statistical analysis details below), which represented editing efficiencies ranging from 22.6±3.8% to 40.0±8.5% of the parental ABE7.10max-NG editing. However, A⋅T to G⋅C editing efficiencies at the HIRA and PSMB2 sites never surpassed 2.4±1.0% by any of the six systems (average A⋅T to G⋅C editing efficiencies by the parental ABE7.10max-NG construct at these two sites were 44.6±18.6% and 45.2±16.9%, respectively, FIG. 1A).

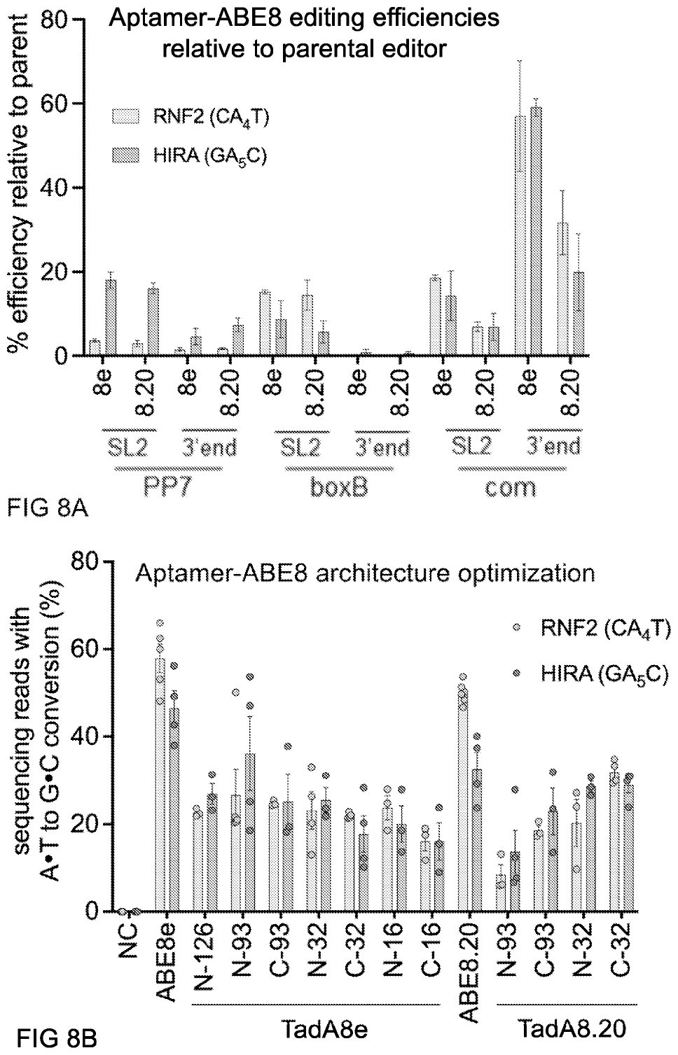

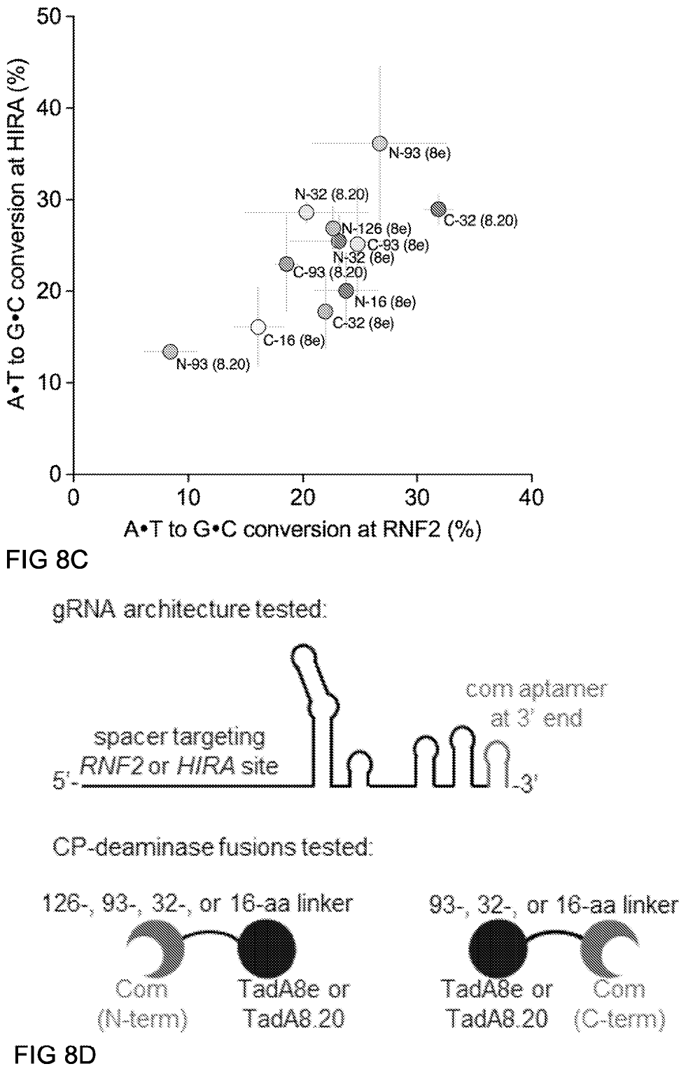

It has been shown that homodimers of later-generation mutant TadA enzymes can decrease DNA editing efficiency compared to their wtTadA-mutant TadA heterodimer counterparts. The dimeric binding mode of PCP to the PP7 aptamer could be causing in trans homodimerization of the mutated TadA7.10 portion of the wtTad-TadA7.10 heterodimers. Therefore, the aptamer systems were expanded to include the boxB and com aptamers, which have smaller CPs (λN, which is 22-aa, and Com, which is 61-aa, respectively) that bind as monomers. These aptamers were embedded into the SL2 or 3′-end of the gRNA (which are closest in 3D space to the target nucleotides) and two poorly edited genomic sites (HIRA and RNF2) were selected on which the engineered constructs were tested. The architecture of the CP-wtTad-TadA-7.10 fusion (N-terminal CP) and linker length (93-aa) constant (FIG. 7B) were held. Overall, it was still observed drastically decreased A⋅T to G⋅C editing efficiencies at both sites compared to the parental ABE7.10max-NG construct (efficiencies ranged from 0.2±0.2% to 10.1±2.1% of the parental construct, FIG. 7B). However, the system that employed the com aptamer embedded at the 3′-end of the gRNA facilitated the highest A⋅T to G⋅C editing efficiencies at both sites (4.7±1.0% at HIRA, and 0.9±0.1% at RNF2, FIG. 8B). Notably, the system with the 3′-boxB gRNA fusion (which performed poorly at both sites; 2.2±2.2% at HIRA, and 0.03±0.03% at RNF2, FIG. 7B) is similar to an aptamer-based ABE system that was recently developed for genome editing in plants, which enabled up to only 8% A⋅T to G⋅C editing at genomic targets in rice.²⁸ This suggests that engineered editing tools may not be transferable across organisms, and may require organism-specific re-engineering efforts to enable efficient editing.

### Example 4

Improving Aptamer-ABE Efficiency with Evolved Deaminases

The wtTadA-TadA7.10 deoxyadenosine deaminase is known to have limited compatibility with alternate Cas enzymes and architectures beyond the parental ABE7.10 construct. To combat this, several eighth-generation TadA enzymes (most notably, TadA8e and TadA8.20) were recently independently evolved, and demonstrate increased editing efficiency, faster deamination kinetics, and enhanced compatibility with additional Cas domains.^{29,30} These deaminase domains may also be more compatible with the aptamer systems described herein. Twelve (12) additional ABE aptamer systems were generated with either PCP, λN, or Com fused to the N-terminus of either TadA8e or TadA8.20 (monomeric deaminase) via a 93-aa flexible linker, and the corresponding aptamers embedded in the SL2 or 3′-end of the Sp-gRNA (FIG. 2C). All combinations of gRNA-aptamers with their cognate CP fused to TadA8 were tested by assessing editing efficiencies at the same two poorly edited sites (HIRA and RNF2). Both evolved TadA8 deaminases displayed improved editing efficiencies when compared to their respective wtTadA-TadA7.10 counterparts with an average increase of 9.6±2.2-fold for TadA8.20 and 15.0±4.3-fold for TadA8e across both sites (up to 44.2±11.1-fold increase was observed for the 3′-end com at RNF2). The systems that employed the com aptamer embedded at the 3′-end of the gRNA again facilitated the highest A⋅T to G⋅C editing efficiencies at both sites and for both TadA8 deaminases (30.7±1.1% at HIRA and 38.3±8.8% at RNF2 for TadA8e, FIG. 2A, and 17.1±3.2% at HIRA and 19.5±4.7% at RNF2 for TadA8.20, FIG. 2B and FIG. 8A). As this aptamer-gRNA construct consistently facilitated the highest editing efficiencies, it was selected for further optimization with both TadA8 deaminases.

The Com-TadA8 fusion architecture (by testing both N- and C-terminal Com fusions) and linker length (by testing flexible linkers taken from established base editors^{25,26} with lengths ranging from 16- to 93-aa) were then optimized to maximize editing efficiency (FIG. 8D). Out of all the aptamer-ABE systems derived from TadA8.20, it was observed the highest editing efficiencies when using a 32-aa linker with Com fused to the C-terminus of TadA8.20 (editing by this construct was 88.7±5.2% of the parental ABE8.20-NG editing efficiency at HIRA and 63.7±2.6% of the parental ABE8.20-NG editing efficiency at RNF2, FIGS. 8C-8D and FIG. 2D). On the other hand, out of all the aptamer-ABE systems derived from TadA8e, it was observed the highest editing efficiencies when a 93-aa linker was used with an N-terminally linked Com (editing by this construct was 77.7±19.3% of the parental ABE8e-NG editing efficiency at HIRA and 46.1±10.4% of the parental ABE8e-NG editing efficiency at RNF2, FIG. 8C and FIG. 2D). These two constructs, termed apt-ABE8e (Com_93-aa_TadA8e, FIG. 2E) and apt-ABE8.20 (TadA8.20_32-aa_Com, FIG. 2E), were therefore both taken forward for characterization.

### Example 5

### Architecture Optimization and an Evolved Deaminase Also Improve Aptamer-CBE Editing Efficiencies in Human Cells

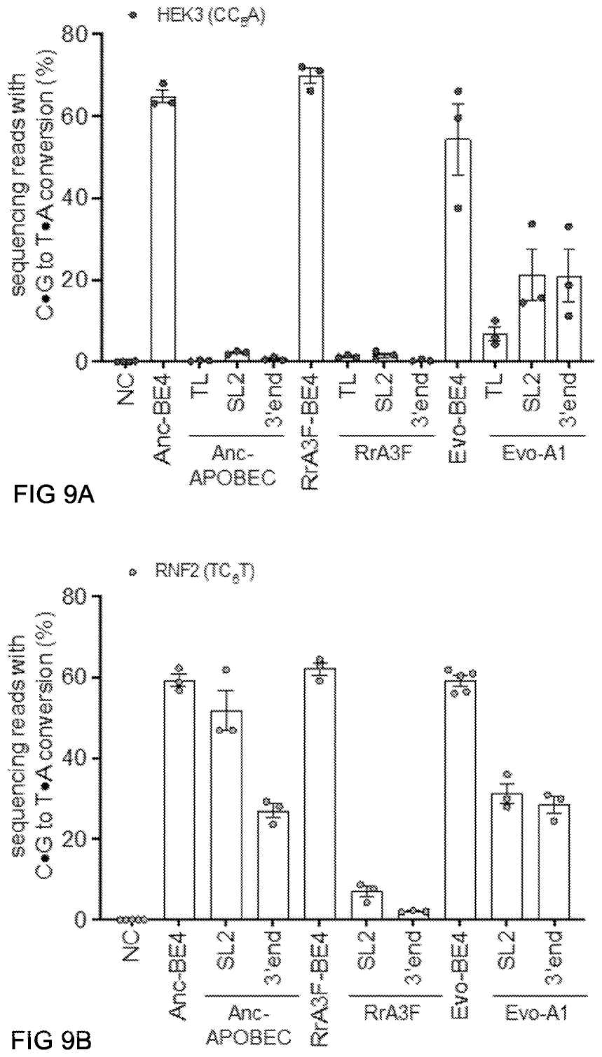

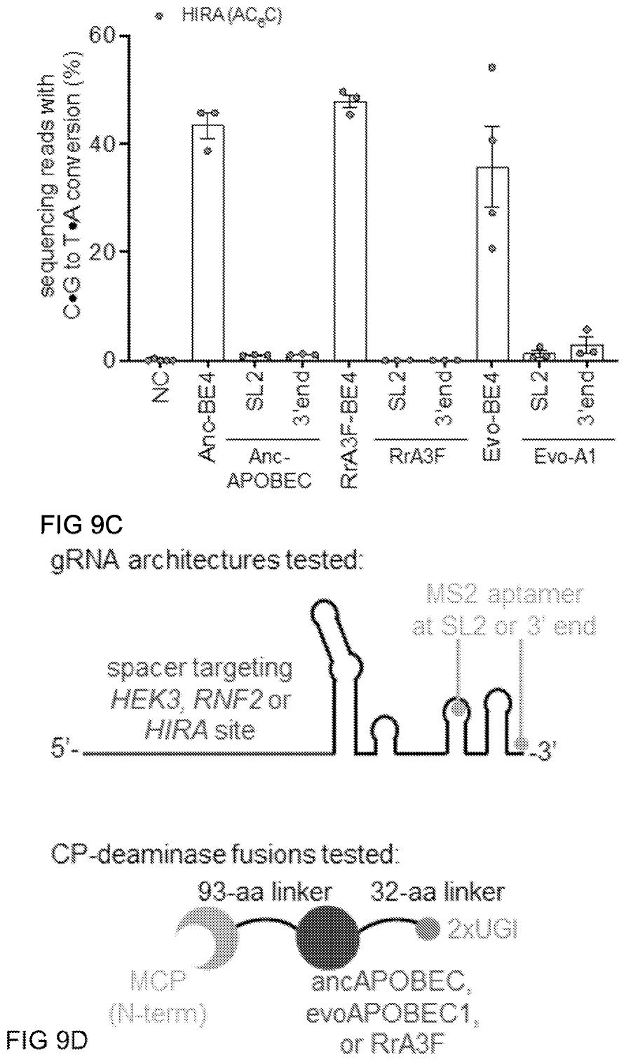

After observing the drastic impact that different deoxyadenosine deaminase variants had on the editing efficiencies of the ABE aptamer systems, three different cytidine deaminase enzymes were selected to be screened for C⋅G to T⋅A editing when used in CBE aptamer architectures. Specifically, ancAPOBEC (an ancestral sequence reconstruction of the rAPOBEC1 enzyme with improved editing activity), evoAPOBECi (an evolved variant of the rAPOBEC1 enzyme with improved editing activity and target sequence compatibility), and RrA3F (a cytidine deaminase identified from a BLAST search with high on-target editing efficiency and low gRNA-independent off-target editing efficiency) were chosen and tested. The MS2-MCP aptamer system was focused on, as it has been used successfully previously with cytidine deaminases. The MS2 aptamer was embedded in the SL2 or 3′-end of the Sp-gRNA and the architecture of the MCP-APOBEC fusion (N-terminal MCP) and linker length (93-aa) constant (FIG. 9D) were held. Editing precision by CBEs is enhanced when the uracil glycosylase inhibitor (UGI) peptide is included in the CBE architecture. Thus, two copies of UGI were appended to the C-terminus of the cytidine deaminase following a flexible 32-aa linker in all constructs (FIG. 9D). Editing efficiencies were then assessed at 3 well-characterized genomic sites (HEK3, HIRA, and RNF2). It was found that the evoAPOBEC1-derived systems most consistently facilitated the highest C⋅G to T⋅A editing efficiencies compared to their ancAPOBEC and RrA3F counterparts, particularly at the poorly edited HEK3 site (editing by the evoAPOBECi systems were on average 30.0±8.8% of the parental evoBE4-NG construct editing efficiency at the HEK3 site, compared to 1.7±0.9% for ancAPOBEC and 1.5±0.5% for RrA3F, FIG. 9A).

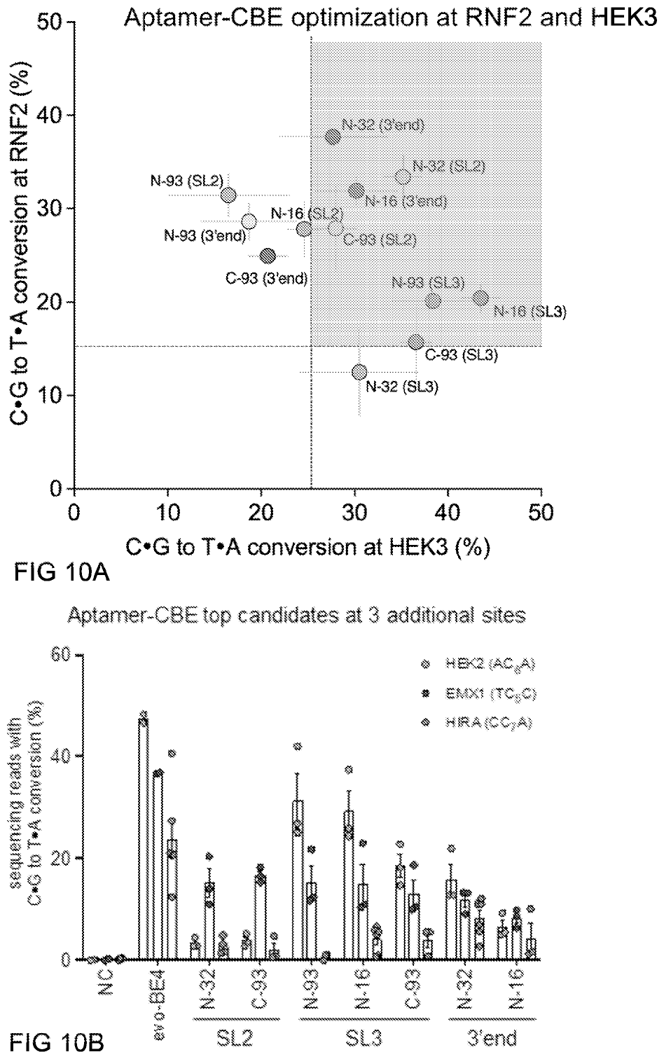

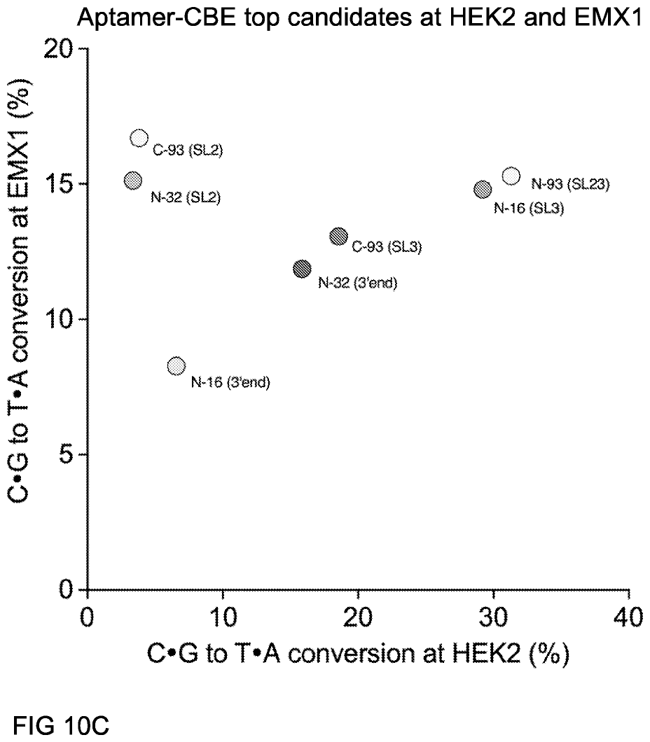

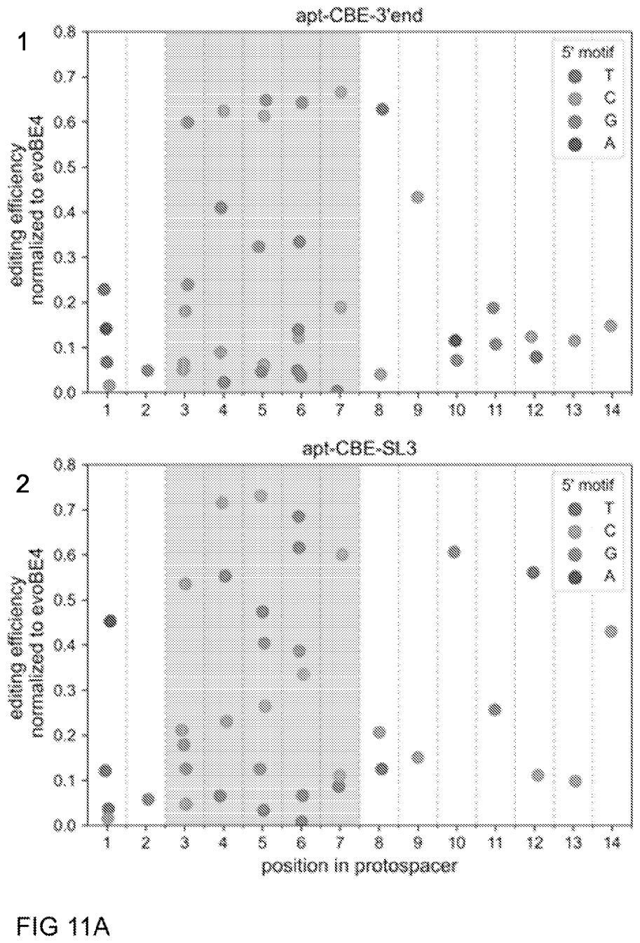

The evoAPOBEC1 deaminase was then studied. Twelve (12) total CBE aptamer systems employing the evoAPOBECi deaminase were generated and their activities were tested at two poorly edited sites (HEK3 and RNF2). The MS2 aptamer was embedded in the SL2, SL3 (it was recently reported that embedding aptamers in the SL3 portion of the gRNA can facilitate recruitment of heterologous effectors [both cytidine and adenosine deaminases concurrently] to the same site for the purposes of “dual base editors”)³, or 3′-end of the Sp-gRNA (FIG. 3C). Both N- and C-terminal MCP-evoAPOBECi fusions with 93-aa linkers, as well as N-terminal MCP-evoAPOBECi fusions with 32- and 16-aa linkers (FIG. 3C), were tested. Although editing efficiencies varied across the three genomic sites, seven constructs displayed C⋅G to T⋅A editing efficiencies of >15% at the RNF2 site and >25% at the HEK3 site (FIG. 3B and FIG. 10A, dotted lines show cutoffs). The editing efficiencies of these seven CBE aptamer systems were further evaluated at additional three genomic loci (EMX1, HIRA, and HEK2). Out of this extended dataset (editing efficiencies at five total sites which had target C's embedded within multiple sequence motifs and positioned at multiple positions within the protospacer, FIG. 3D), it was found that apt-CBE-3′end (MS2 embedded in the 3′-end of the gRNA with the MCP fused to the N-terminus of evoAPOBEC1 via a 32-aa linker, FIG. 3E) and apt-CBE-SL3 (MS2 embedded in SL3 of the gRNA with the MCP fused to the N-terminus of evoAPOBECi via a 16-aa linker, FIG. 3E) displayed the most consistently high C⋅G to T⋅A editing efficiencies (editing by the apt-CBE-3′end system ranged from 28.0±5.1% to 63.8±1.5% [average 43.1±6.4%] of the parental evoBE4-NG construct across all sites, and editing by the apt-CBE-SL3 system ranged from 14.0±3.8% to 69.3±3.2% [average 44.7±9.7%] of the parental evoBE4-NG construct across all sites, FIG. 3D and FIGS. 10A-10C). These constructs as the final CBE aptamer systems were further characterized in Example 5 below.

### Example 6

### Characterization of Aptamer-BEs at Genomic Sites in Human Cells

Each of the four optimized BE aptamer systems was further characterized by measuring their base editing efficiencies at 15 genomic protospacers, which collectively contained 76 C and 116 A nucleotides spanning all positions 1-20 within the protospacer. Each BE aptamer system was analyzed for editing efficiency, window size, and sequence preference. All four aptamer systems displayed reduced editing efficiencies compared to their respective parental constructs. Specifically, editing by apt-CBE-3′end averaged 27.1±6.6% of the parental evoBE4, editing by apt-CBE-SL3 averaged 28.6±6.0% of the parental evoBE4, editing by apt-ABE8e averaged 54.4±6.6% of the parental ABE8e, and editing by apt-ABE8.20 averaged 52.0±5.0% of the parental ABE8.20, FIGS. 4C-D.

To quantify the editing window, an average editing efficiency at each position (which was averaged across all measured Cs or As at that position) was first computed for each editor (shown as the bars in FIGS. 4A-4B). The “editing window” was defined as the positions within the protospacer which have average editing efficiencies that exceed 25% of the maximal average editing efficiency observed for a given editor. All four aptamer systems displayed slightly narrower editing windows than their parent editors. The parental evoBE4 editor displayed an editing window of positions 3 through 8, while both apt-CBE-3′end and apt-CBE-SL3 displayed editing windows of 3 through 7 (FIG. 4A). The parental ABE8e editor displayed an editing window of positions 3 through 8, while apt-ABE8e displayed an editing window of positions 4 through 7 (FIG. 4B). Finally, the parental ABE8.20 editor displayed an editing window of positions 3 through 7, while apt-ABE8.20 displayed an editing window of positions 4 through 7 (FIG. 4B). No editing above background for any of the aptamer systems outside of the protospacers was observed.

All three parental editors displayed minimal sequence context preferences, consistent with previous reports.^{29,30,32} For each aptamer system, the average editing efficiency was normalized for each individual C or A to the average editing efficiency for that base by the corresponding parental editor (FIGS. 11A-11B). It was found the apt-CBE-3′end construct prefers a 5′ T or C motif (all of the C's with >50% activity compared to the parental system had this sequence motif). The apt-CBE-SL3 construct, in contrast, had a 5′ A or C motif preference. The apt-ABE8e construct exhibited an aversion to the 5′ A motif, while the apt-ABE8.20 construct did not appear to have any sequence motif preferences.

### Example 7

### Combining Aptamer-BEs to Make a Multiplexed Orthogonal Base Editing (MOBE) System

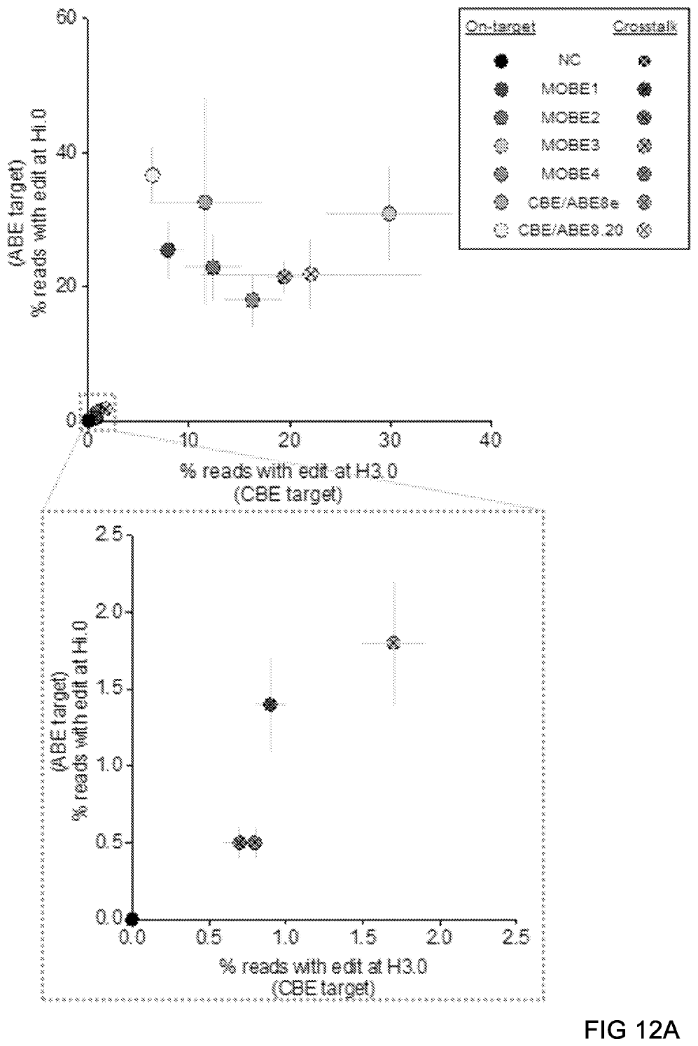

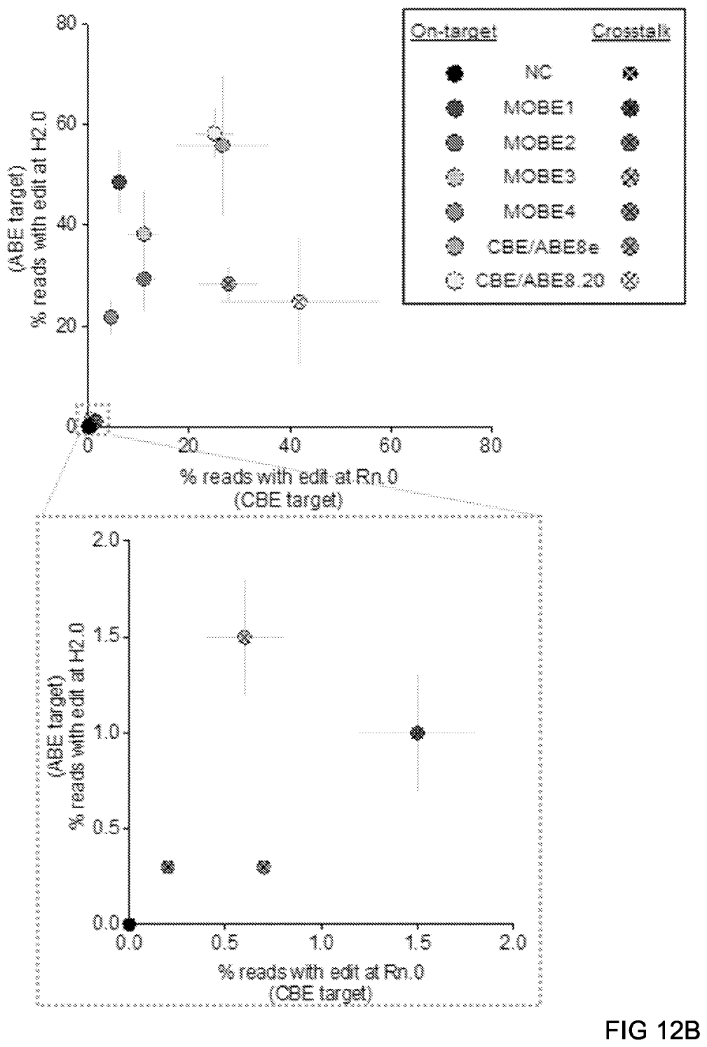

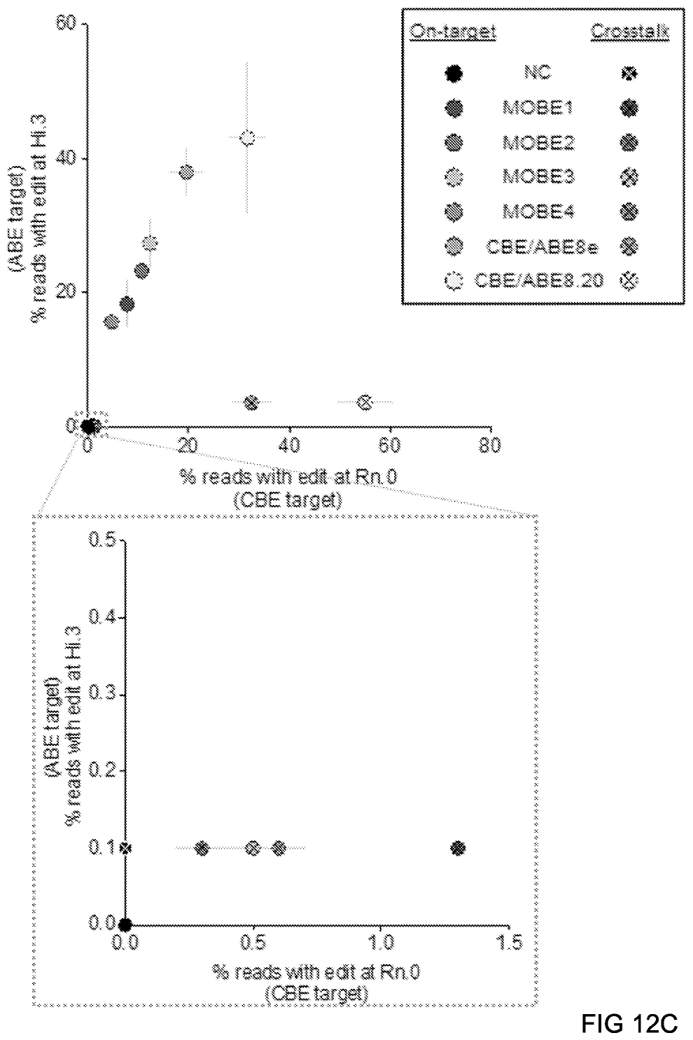

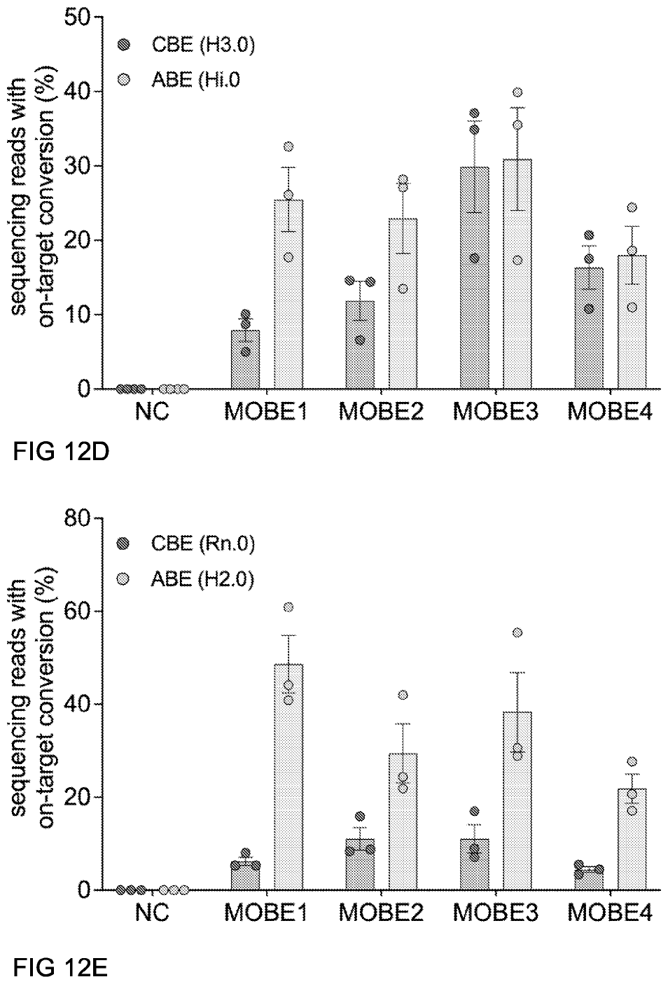

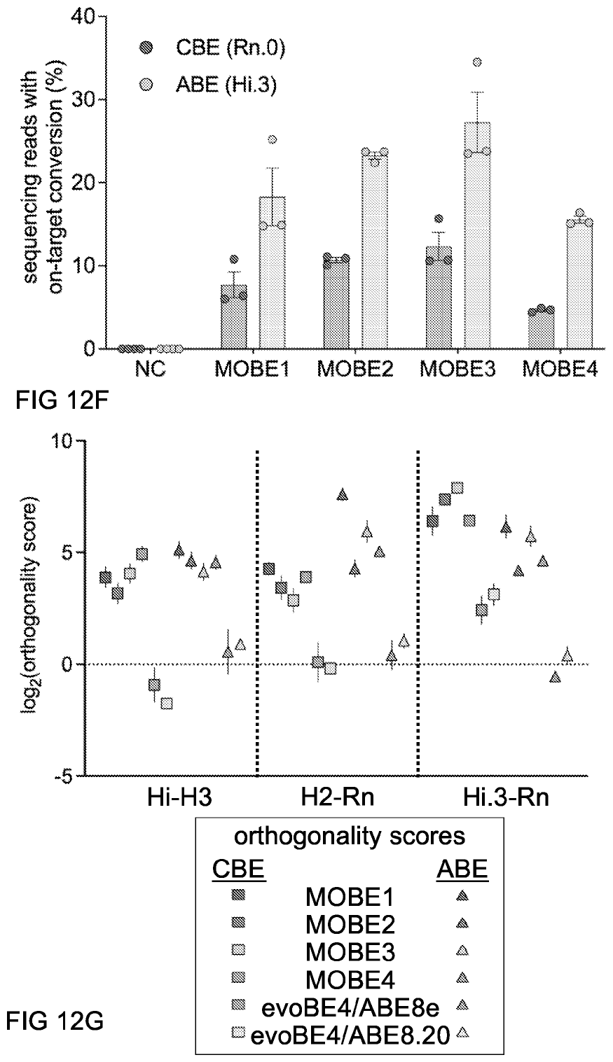

Four multiplexed orthogonal base editor (MOBE) systems were generated by combining each optimized CBE aptamer system (apt-CBE-3′end and apt-CBE-SL3) with each optimized ABE aptamer system (apt-ABE8e and apt-ABE8.20). For each MOBE system (which was labelled MOBE1-4, in accordance with FIG. 5A), the two CP-deaminase constructs were joined with a tandem PT2A linker (which robustly induces ribosomal skipping, ensuring that no dual deaminase fusion protein is produced). These effectors were simultaneously targeted to distinct genomic sites using two plasmids expressing their respective com or MS2 aptamer-gRNAs. Cells were transfected with plasmids encoding nCas9-NG-P2A-mCherry, tandem CP-deaminase fusions, and both gRNA-aptamers, lysed three days after transfection, and editing was quantified by NGS. Editing was measured at three different protospacer combinations (HIRA.0/HEK3.0, HEK2.0/RNF2.0, and HIRA.3/RNF2.0, where in each combination the ABE target is listed first, and the CBE target is listed second). Multiplexed editing with the parental, non-orthogonal ABE and CBE constructs (the evoBE4-NG parental CBE construct was multiplexed with either ABE8e-NG or ABE8.20-NG using unmodified gRNAs targeting the same protospacers as the MOBE systems) was also performed. The MOBE systems displayed high levels of on-target activity across all target combinations compared to their parental counterparts; the MOBE3 system averaged 25.0±4.5% editing (which corresponded to an average 17.7±6.1% C⋅G to T⋅A conversion and an average 32.2±3.2% A⋅T to G⋅C conversion). This is compared to its parental evoBE4-NG/ABE8e-NG system, which displayed an average on-target editing efficiency of 30.7±6.3% (which corresponded to an average 19.3±4.3% C⋅G to T⋅A conversion across the CBE target sites, and an average 42.2±7.0% A⋅T to G⋅C conversion across the ABE target sites). Importantly, the MOBE systems showed greatly decreased crosstalk (non-orthogonal edits) compared to the parental constructs. MOBE1, which had the lowest crosstalk editing, averaged 0.36±0.09% crosstalk editing across all protospacers, and MOBE3, which had the highest crosstalk editing, averaged 1.0±0.3% crosstalk editing (FIG. 5B and FIGS. 12A-12G, dotted line insets). In contrast, the evoBE4-NG/ABE8e-NG and evoBE4-NG/ABE8.20-NG parental systems averaged 28.2±7.3% and 22.2±4.2% crosstalk editing, respectively (FIG. 5B and FIGS. 12A-12G). Specifically, when targeted to the HIRA.0/HEK3.0 loci, MOBE3 had the highest on-target activity, with 30.9±6.9% A⋅T to G⋅C editing at the ABE target (HIRA.0) and 29.9±6.2% C⋅G to T⋅A editing at the CBE target (HEK3.0), with less than 1.8±0.4% crosstalk at both sites (FIG. 5B). This is compared to the parental evoBE4-NG/ABE8e-NG combination, which had 32.6±15.4% A⋅T to G⋅C editing at the ABE target (HIRA.0) and 11.6±5.5% C⋅G to T⋅A editing at the CBE target (HEK3.0), with 22.1±11.0% C⋅G to T⋅A crosstalk at the HIRA.0 site and 21.9±5.2% A⋅T to G⋅C crosstalk at the HEK3.0 site (FIG. 5B).

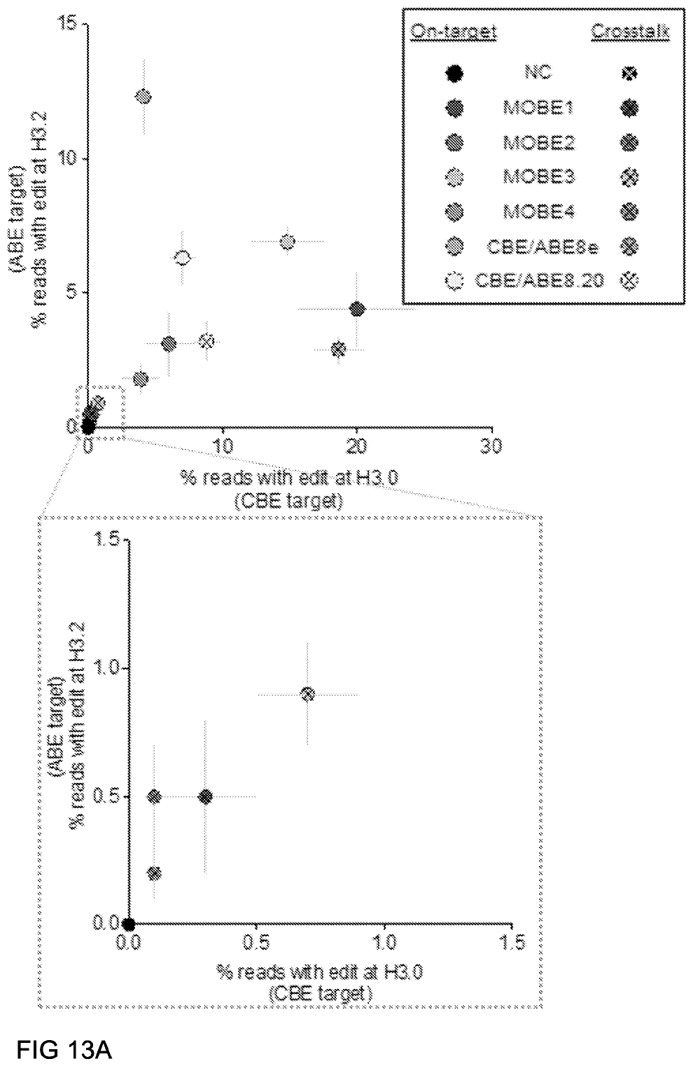

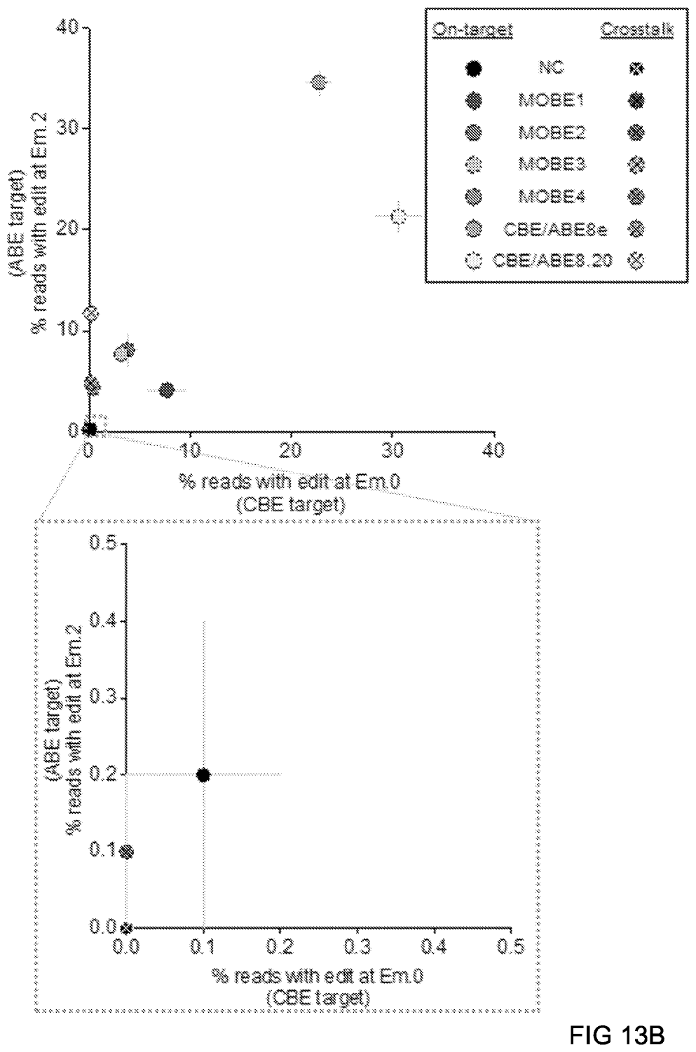

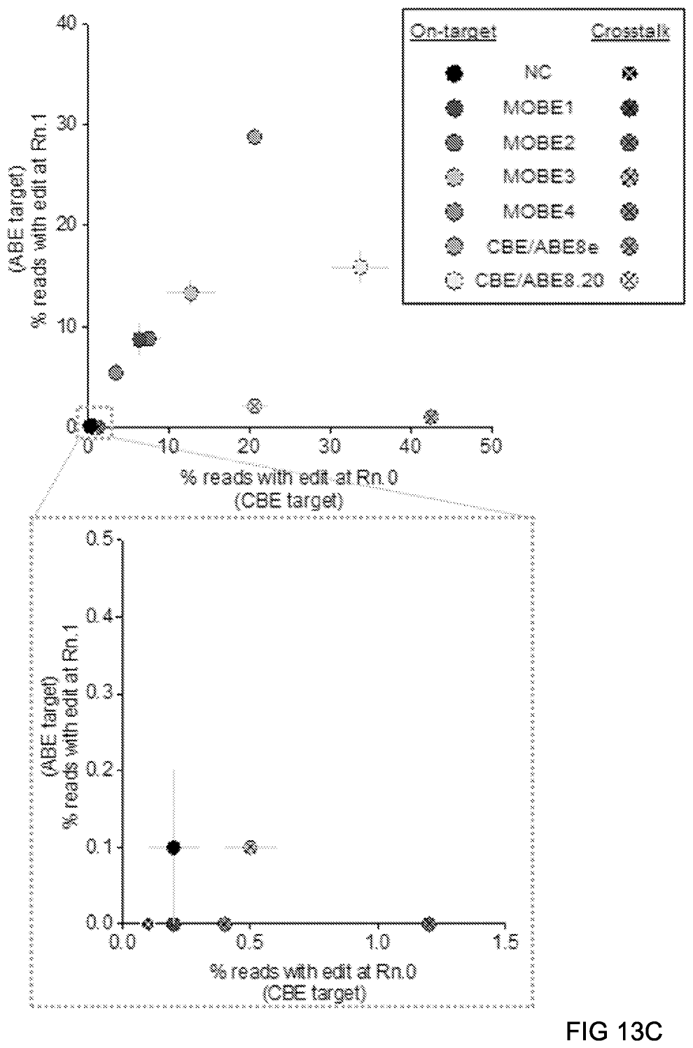

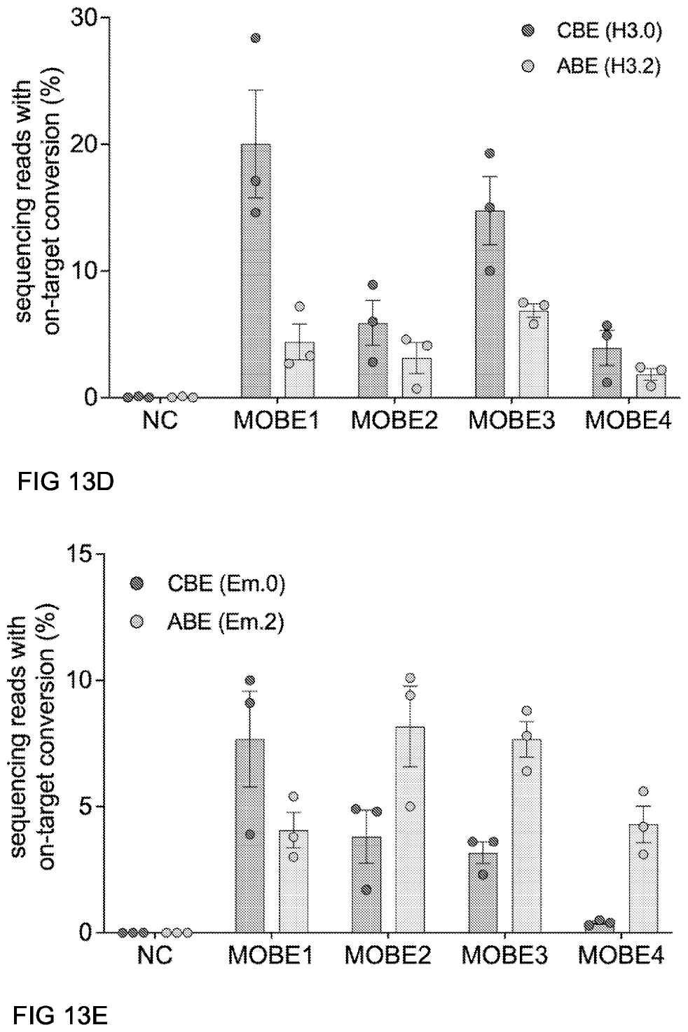

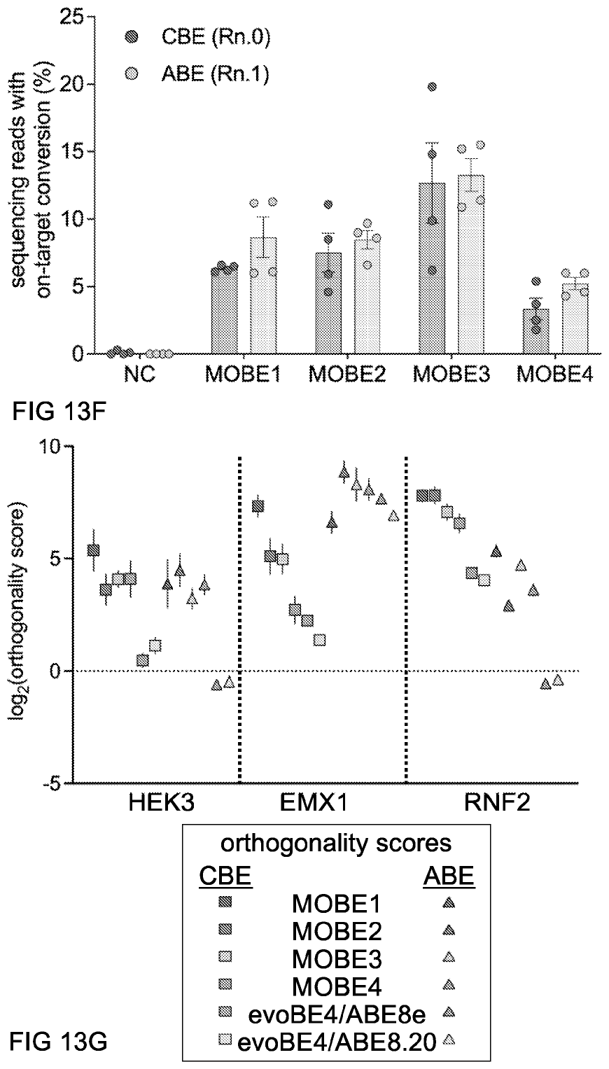

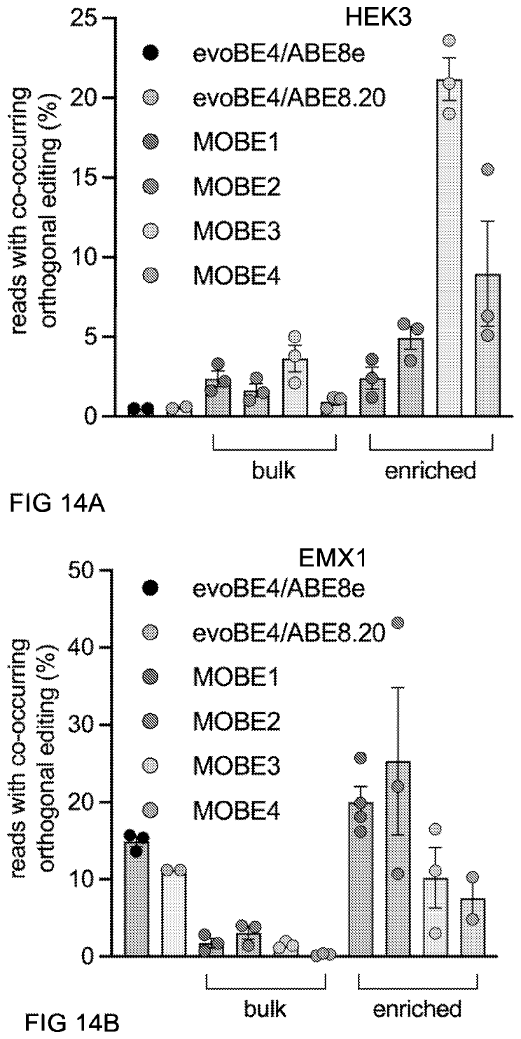

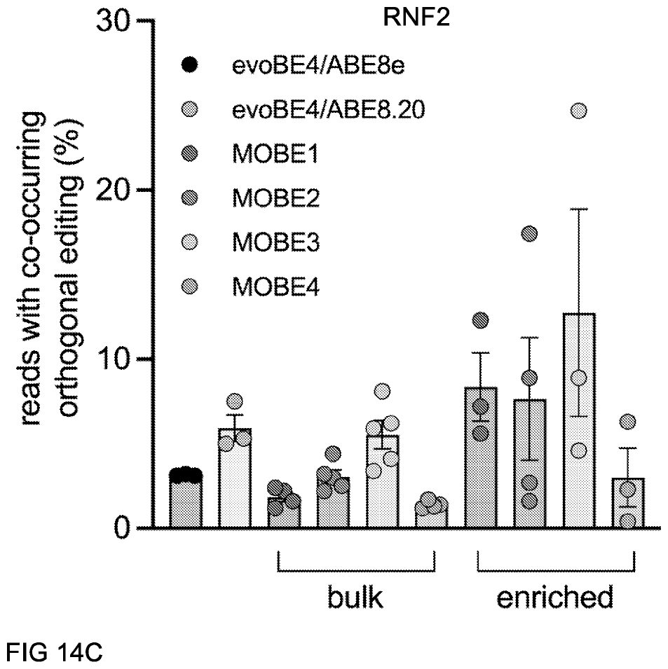

Next, two protospacers within a single genomic region to represent modelling tandem SNVs, such as those inherited in haplotype blocks or observed in cis-regulatory elements, were targeted. This approach also allows the quantification of co-occurring point mutations by targeted amplicon NGS. Three protospacer combinations (at the EMX1, HEK3, and RNF2 loci) were selected and multiplexed orthogonal editing efficiencies were quantified at two protospacers within each locus by the four MOBE systems, as well as the two parental non-orthogonal systems. While lower levels of on-target activity by MOBEs were generally observed as compared to their parental counterparts at these three site combinations (particularly the EMX1 combination, in which all four MOBEs facilitated less than 10% editing at both protospacers), significantly decreased crosstalk editing was again detected as compared to the parental constructs. The crosstalk editing for the MOBE systems averaged 0.3±0.1% (ranging from 0.016±0.007% to 1.2±0.1%) compared to the parental systems that averaged 9.7±2.5% crosstalk editing (ranging from 0.18±0.02% to 42.5±0.8%, FIG. 5C and FIGS. 13A-13G, dotted line insets). The highest on-target editing was typically facilitated by MOBE3 or MOBE1 (the system with the absolute highest on-target activity was site-dependent), followed by MOBE2 and then MOBE4. Specifically, at the HEK3 site combination (PAM-out orientation, 168 bp apart), MOBE3 introduced on-target editing efficiencies of 14.8%±2.7% C⋅G to T⋅A and 6.9±0.5% A⋅T to G⋅C (as assessed in bulk), with crosstalk editing of 0.9±0.2% C⋅G to T⋅A and 0.7±0.2% A⋅T to G⋅C. In contrast, the parental evoBE4-NG/ABE8e-NG combination introduced on-target editing efficiencies of 4.1±0.6% C⋅G to T⋅A and 12.3±1.4% A⋅T to G⋅C (as assessed in bulk), with crosstalk editing of 2.9±0.5% C⋅G to T⋅A and 18.6±1.9% A⋅T to G⋅C.

The “CBE orthogonality score” of an editing system for a given protospacer combination was defined as the ratio of the on-target C⋅G to T⋅A editing efficiency to the crosstalk C⋅G to T⋅A editing efficiency, with the “ABE orthogonality score” being the A⋅T to G⋅C editing efficiency equivalent. Thus, higher scores correspond to higher orthogonality, while scores near 1 represent equal levels of on-target and crosstalk activity. The MOBE systems displayed a median CBE orthogonality score of 31.0, which ranged from 6.6±2.7 to 239±41 for the six total protospacer combinations tested (FIG. 5D). The median ABE orthogonality score of the MOBEs was 25.5, with individual ABE orthogonality scores ranging from 7.6±1.0 to 460±160. (FIG. 5D). The parental evoBE4-NG/ABE8e-NG and evoBE4-NG/ABE8.20-NG combinations displayed a median CBE orthogonality score of 2.4 (which ranged from 0.30±0.03 to 20.3±1.0), and a median ABE orthogonality score of 1.3 (ranging from 0.7±0.1 to 203±19, FIG. 5D). When directly comparing each MOBE's orthogonality score to its parental orthogonality score for the same protospacer combination, the four MOBE systems were found to collectively be on average 24.9±4.2-fold more orthogonal than the parental systems. Finally, the MOBE1 system typically displayed the highest orthogonality scores, with median CBE and ABE orthogonality scores of 63.0 and 55.9, respectively.

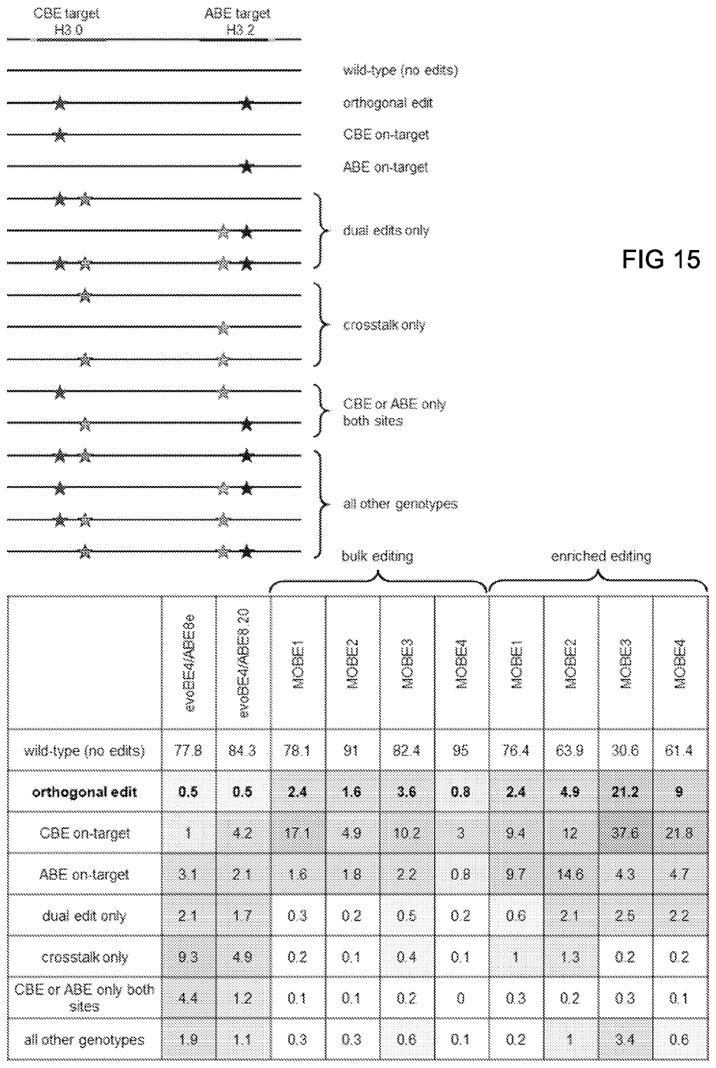

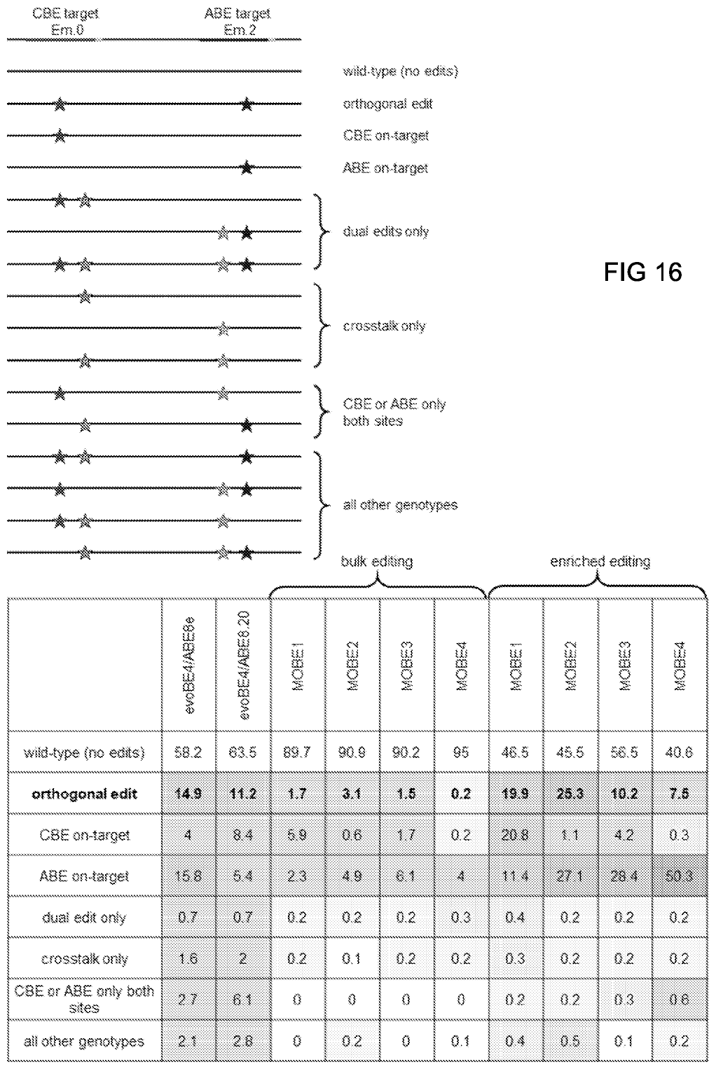

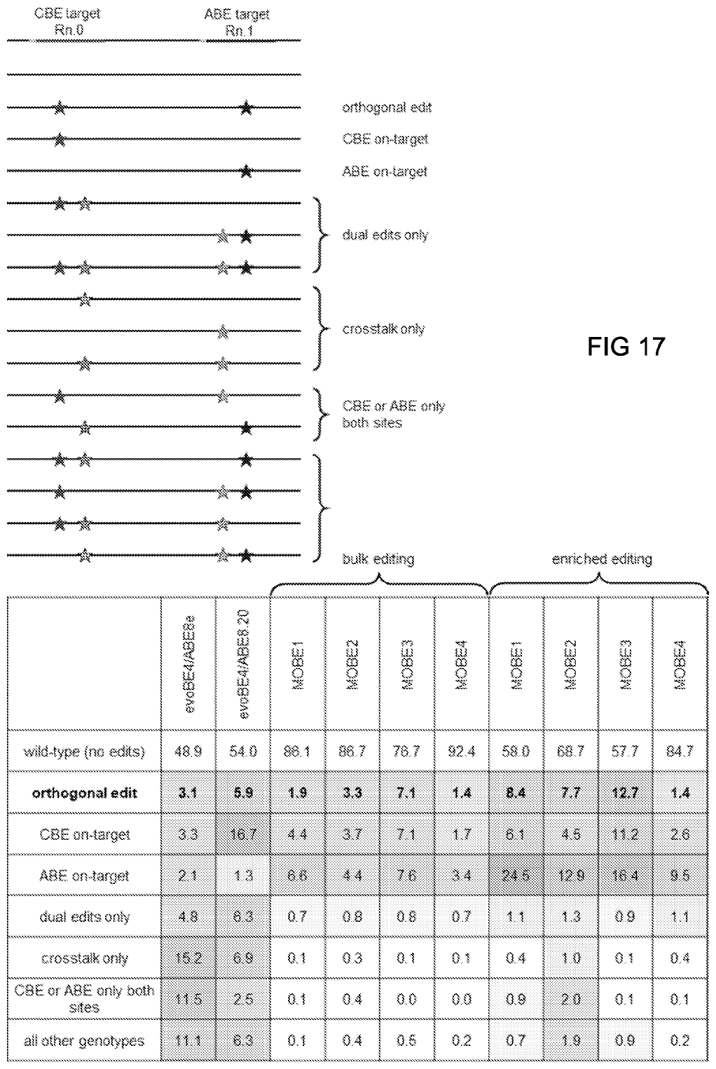

The genotypes of the three single-amplicon targets (FIGS. 15-17) were also analyzed with a particular interest in the percent of reads with simultaneous orthogonal on-target edits at both protospacers (which referred to as reads with co-occurring orthogonal edits, FIGS. 15-17). It was found that at the HEK3 and RNF2 combinations (at which the MOBE systems typically displayed bulk editing efficiencies >30% of the parental systems), MOBE3 generated the largest percent of reads with co-occurring orthogonal edits, while MOBE4 generated the lowest. In particular, at the HEK3 site combination (of which both protospacers contain both a highly edited A and C base, FIG. 13A), 3.6±0.8% of reads from the MOBE3-treated cells contained co-occurring orthogonal edits, compared to 0.5±0.0% for its evoBE4-NG/ABE8e-NG parental editor. At the RNF2 site combination (of which the RNF.1 ABE target protospacer lacks a highly edited C, making this site more naturally prone to orthogonality, FIG. 13C), 7.1±0.5% of reads from the MOBE3-treated cells contained co-occurring orthogonal edits, compared to 3.1±0.0% for its evoBE4-NG/ABE8e-NG parental editor, and 5.9±0.8% for the evoBE4-NG/ABE8.20-NG parental editor. However, at all sites it was observed drastic decreases in the percent of reads corresponding to genotypes with crosstalk edits compared to the parental editors, even when normalized to overall editing efficiencies (FIGS. 15-17). Specifically, at the RNF2 combination, despite having similar absolute levels of co-occurring orthogonal edits, only 6.1±0.3% of edited reads had genotypes with crosstalk for MOBE3, while 83.3±0.3% of reads from cells treated with the evoBE4-NG/ABE8e-NG parental system had crosstalk genotypes, demonstrating the enhanced precision of the MOBE systems. The EMX1 combination was also naturally prone to orthogonality (the EMX1.0 CBE target protospacer lacks an efficiently edited A, FIG. 13B), and the MOBE systems had significantly decreased on-target editing efficiencies at this protospacer combination compared to the parental systems. Therefore, a strategy to enrich for co-occurring orthogonal edits was further sought to develop.

### Example 8

all-In-One Fluorescent Reporter Plasmid Enriches for Co-Occurring Point Mutations by MOBEs

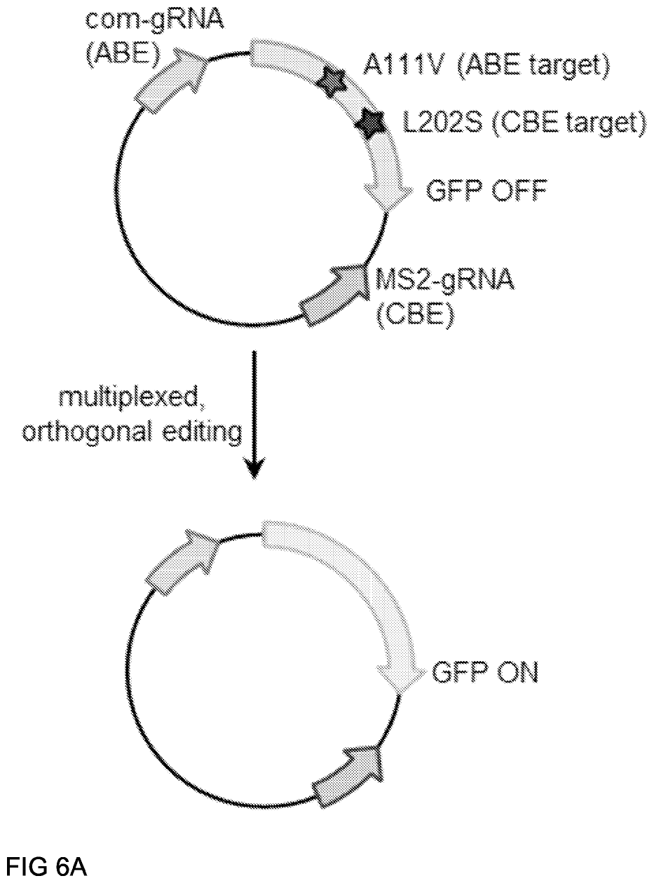

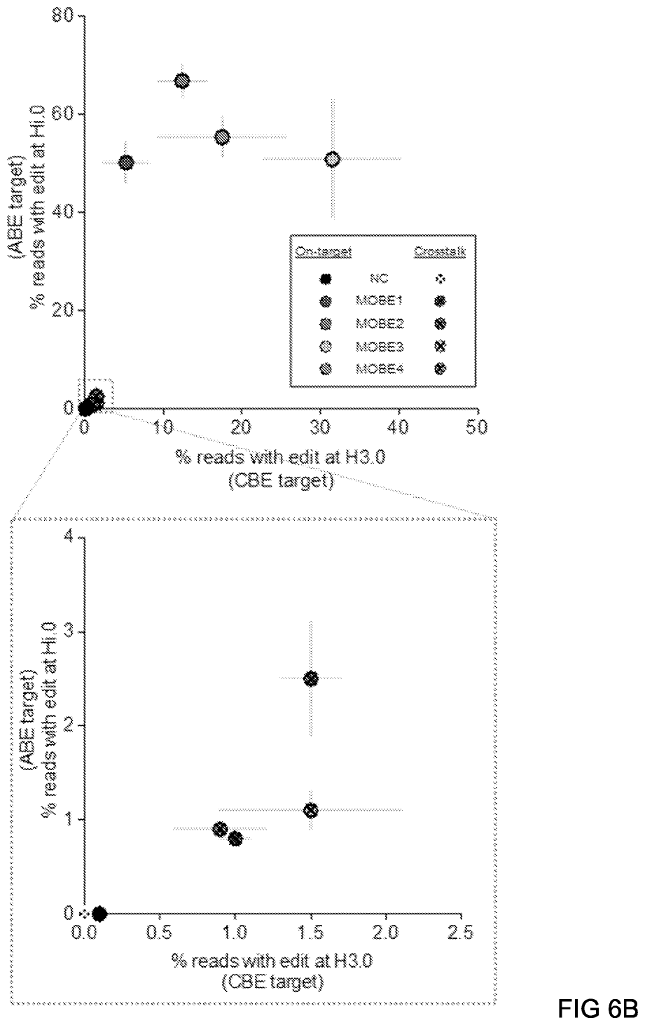

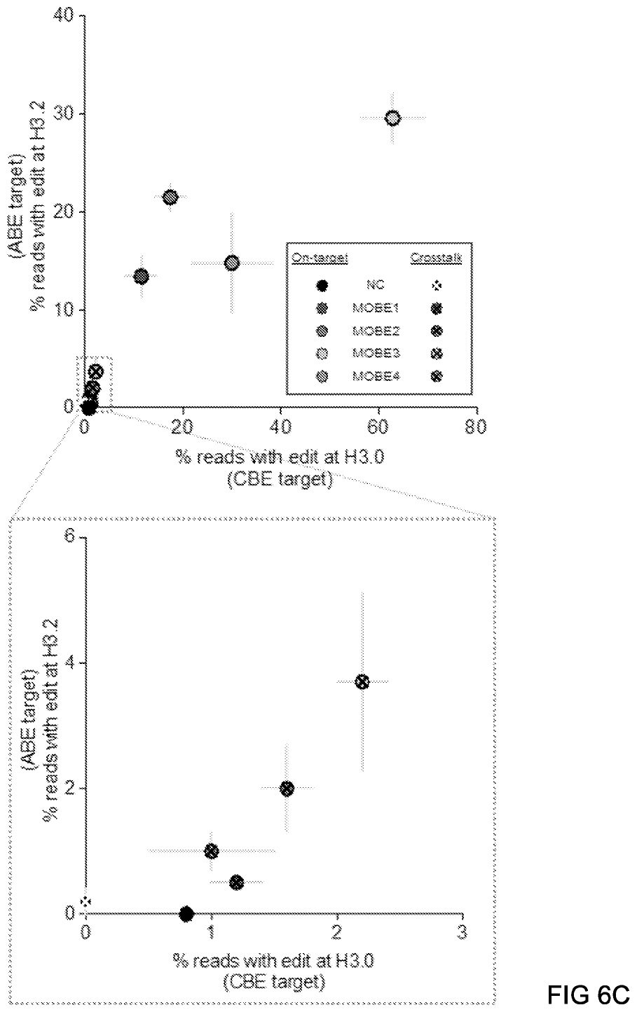

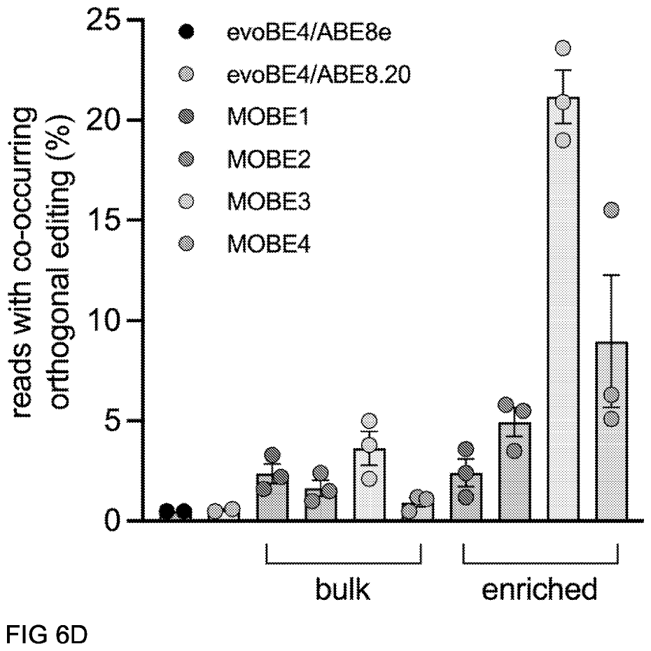

Because the MOBE systems exhibited lower on-target activity than their parental counterparts, a plasmid-based enrichment system was designed to enrich for orthogonal editing activity when co-transfected with genomic-targeting gRNAs. The reporter plasmid encodes a “2×-dead-GFP” gene (which harbors two missense mutations that require orthogonal A⋅T to G⋅C and C⋅G to T⋅A editing to produce functional, fluorescent GFP), as well as two gRNA-aptamer fusions targeted to the appropriate locations within the “2×-dead-GFP” gene (FIG. 6A). HEK293T cells were transfected with MOBEs targeted to the same six genomic site combinations used previously, and the reporter plasmid.

After 96 hours, populations of both “unenriched” cells and GFP+/mCherry+“enriched” cells were collected by fluorescence activated cell sorting (FACS) and editing efficiencies were determined by NGS. Both GFP-targeting protospacers had both a target A and C within the editing window, and crosstalk editing would result in additional amino acid changes to the GFP protein. This precluded the use of this enrichment system with the parental systems.

MOBE3 typically exhibited the highest enriched editing, averaging 27.4±7.6% on-target C⋅G to T⋅A and 47.4±7.7% on-target A⋅T to G⋅C editing across the six protospacer combinations (FIG. 6C and FIGS. 18A-18F and FIGS. 19A-19F). The highest C⋅G to T⋅A editing observed was 62.9±6.4% by MOBE3 at the HEK3.0 protospacer (FIG. 19A), and the highest A⋅T to G⋅C editing we observed was 81.1±1.2% by MOBE2 at the HEK2.0 protospacer (FIG. 18B). Increases in editing efficiencies ranging from 4.9±2.5 to 33.6 ±19.6-fold for MOBE3 were observed when comparing enriched cells to unenriched cells from the same sample (FIG. 20A), clearly demonstrating the utility of this enrichment strategy. It is important to note that lower editing efficiencies in unenriched cells were observed in these experiments as compared to the previous bulk editing MOBE experiments, likely due to an extra expansion step that was employed to ensure there would be enough enriched cells for NGS analysis. Importantly, this increase in editing efficiency was not accompanied by a decrease in orthogonality scores; both ABE and CBE orthogonality scores for a given protospacer combination was within error of unenriched samples (FIG. 20B).

The genotypes of the three single amplicon target sites were again analyzed and up to a 37.5-fold increase was observed in the percent of reads with co-occurring orthogonal edits compared to bulk samples. At all three site combinations, at least two MOBE systems demonstrated higher absolute co-occurring orthogonal editing percentages than both parental systems (FIGS. 15-17). The highest absolute rate of co-occurring orthogonal edits was 25.3±9.7%, facilitated by MOBE2 at the EMX1 site (previously, co-occurring orthogonal editing rates was the lowest at this site). When normalized to overall editing efficiencies, MOBE2 at the EMX1 site combination had the highest precision as well, with 46.4±14.1% of edited reads containing the desired genotype (co-occurring orthogonal edits). MOBE3 facilitated the highest rates of co-occurring edits at the other two sites (21.2±1.3% at the HEK3 combination, and 12.7±6.1% at the RNF2 combination), with >30% of edited reads containing the desired genotype at both sites (co-occurring orthogonal edits). Consistent with the stable orthogonality scores, statistically significant increases in the percent of edited reads with crosstalk editing by the MOBE systems upon enrichment were observed. Consequently, all MOBE systems at all three single-amplicon targets demonstrated lower rates of edited reads with crosstalk genotypes compared to their respective parental systems.

Collectively, the MOBE systems combined with this enrichment strategy facilitated up to a 42.4±3.7-fold increase in the absolute rate of co-occurring orthogonal edits compared to their respective parental systems (MOBE3 at the HEK3 site). Furthermore, it was observed up to 16.8±6.1-fold lower rates of edited reads with crosstalk edits of the MOBE systems compared to their parental systems. These data clearly indicate that the MOBE reporter plasmid is an effective option for enriching for cells harboring co-occurring orthogonal edits. This strategy is crucial when utilizing MOBEs to generate cell lines, at poorly edited targets, or in difficult-to-edit cell types.

### Example 9

MOBE Compatibility with Other nCas9 Variants

As the MOBE systems are modular, they can be easily modified to implement additional Sp-nCas9 variants. Specifically, the targeting scope can be expanded by using the near PAM-less SpRY nCas9 variant, which can be especially useful given the relatively narrow MOBE editing window.¹⁵ Additionally, MOBEs can be used with high-fidelity nCas9 variants such as HiFi to mitigate gRNA-dependent off-target effects.⁴⁷ In this Example, HEK293T cells were transfected with plasmids encoding tandem CP-deaminase fusions, both gRNA-aptamers, and either nCas9-NG-P2A-mCherry, SpRY-nCas9-P2A-mCherry, or HiFi-nCas9-P2A-mCherry. Cells were lysed after three days, and editing was quantified by NGS. Editing was evaluated at the RNF2 single amplicon protospacer combination and the H/RA.0/HEK3.0 protospacer combination (FIGS. 21A-21B). For all MOBE systems, efficient on-target C⋅G to T⋅A and A⋅T to G⋅C editing efficiencies were observed for both the SpRY and HiFi nCas9 variants, with low crosstalk editing efficiencies (FIGS. 21A-21B). These data demonstrate that all MOBE systems are compatible with additional Sp-nCas9 variants.

### Example 10

Off-Target Analysis of MOBEs Compared to their Parental BEs

The off-target editing propensities of the four MOBE systems were then evaluated and compared to the parental, non-orthogonal combinations. First, the gRNA-independent off-target DNA editing activities of all systems were evaluated using an orthogonal R-loop assay (FIG. 22E).^{45,48} In this assay, a Sa-dCas9 is programmed to bind to a genomic locus of interest and forms an R-loop but does not modify the DNA. Concurrently, Sp-Cas9-derived BEs were co-transfected, and if the deaminase components of the BEs have gRNA-independent off-target editing activity, they deaminate target Cs or As within the Sa-dCas9-exposed R-loop. In this Example, HEK293T cells were transfected with one of the four MOBE systems, as well as plasmids encoding Sa-dCas9 and Sa-gRNA. After three days, the cells were lysed and editing at the Sa-targeted genomic locus was quantified by NGS. The studies with the parental, non-orthogonal ABE and CBE constructs (the evoBE4-NG parental CBE construct was multiplexed with either ABE8e-NG or ABE8.20-NG using unmodified gRNAs) were also performed. The off-target editing efficiencies were evaluated at four different Sa-targeted genomic loci that contain both target Cs and As within the exposed R-loop (labelled as Sa-site 1 through Sa-site 4). At all four sites, gRNA-independent off-target editing of both Cs and As was highest by MOBE1, followed by MOBE3, MOBE2, and MOBE4 (FIGS. 22A-22D). gRNA-independent off-target editing of Cs by all four MOBE systems (which share the same cytidine deaminase domain as both parental systems) was equivalent or lower than that of the parental constructs (FIGS. 22A-22D). gRNA-independent off-target editing of As by MOBE1 and MOBE3 (which share the same adenosine deaminase domain as the parental ABE8e construct) was significantly lower than that of the parental evoBE4/ABE8e combination, and gRNA-independent off-target editing of As by MOBE2 and MOBE4 (which share the same adenosine deaminase domain as the parental ABE8.20 construct) was within error of that of the parental evoBE4/ABE8.20 combination.

The gRNA-dependent off-target editing of the four MOBE systems was further evaluated and compared to the parental systems. Two of the most efficiently edited protospacer combinations (the RNF2 single amplicon protospacer combination and the HIRA.0/HEK3.0 protospacer combination) were chosen and at least two putative off-target sites for each of the four protospacers were identified. The HEK3.0 protospacer has been previously experimentally evaluated for off-target editing;⁴⁹ the top three off-target sites were chosen for evaluation. For the other three protospacers, the cutting frequency determination (CFD) score calculation was used to identify the top two putative off-target sites and evaluated editing at these two sites.⁵⁰ HEK293T cells were transfected with one of the four MOBE systems or the parental ABE/CBE combinations, lysed after three days, and the off-target genomic loci were amplified and sequenced with NGS. All four MOBEs were evaluated using nCas9-NG and the HiFi-nCas9 variant. Editing above background levels was observed only at one of the HIRA off-target loci, and it was found that all four MOBE systems (both the MOBE systems utilizing the nCas9-NG variant as well as the HiFi-nCas9 variant) displayed off-target A⋅T to G⋅C editing efficiencies within error of their respective parental systems (FIGS. 23A-23B).

Further, unguided off-target RNA editing of the four MOBE systems was also evaluated and compared to the parental systems. Three highly expressed RNA transcripts that have previously been used to evaluate unguided off-target RNA editing by BEs (CTNNB1, IP90, and RSL1D1)⁵¹ were chosen. In this study, HEK293T cells were transfected with MOBEs or the parental ABE/CBE combinations, extracted total RNA after 48 hours, reverse-transcribed the mRNA into cDNA, and sequenced the three transcriptomic sites of interest. Both A to I and C to U off-target RNA editing was evaluated via three different methods; the average A to I or C to U conversion among all As or Cs within the transcript, the maximal A to I or C to U conversion among all As or Cs within the transcript, and the number of As or Cs within the transcripts with A to I or C to U conversions greater than 0.1%. By all three metrics and within all three transcripts, the gRNA-independent off-target A to I RNA editing activities of all four MOBE systems were within error or lower than their respective parental systems (FIG. 23C). gRNA-independent off-target C to U editing values within the/P90 transcript were within error of negative controls, and only the evoBE4/ABE8.20 combination displayed off-target C to U editing values above negative controls within the CTNNB1 transcript, as evaluated by all three methods. Within the RSL1D1 transcript, it was observed that the four MOBE systems (which share the same cytidine deaminase domain as both parental systems) displayed gRNA-independent off-target C to U editing values in between those of the two parental systems (FIG. 23D).

### Example 11

MOBE Compatibility with Other Cell Types

The MOBE systems in additional mammalian cell types were also evaluated. MOBE2 and MOBE3 (which employ different adenosine deaminase domains) were selected to be evaluated in HeLa cells at the RNF2 single amplicon protospacer combination and the HIRA.0/HEK3.0 protospacer combination. HeLa cells were transfected with either of the two MOBE systems and the GFP enrichment plasmid. After 72 hours, GFP+/mCherry+cells were collected by FACS, and editing efficiencies were determined by NGS. Similar trends were observed in the relative on-target editing efficiencies of MOBE3 compared to MOBE2, and in general, it was observed on-target editing efficiencies that are roughly 50% of those observed at the same genomic loci in HEK293T cells following this enrichment strategy (FIGS. 24A-B). These decreases in on-target editing efficiencies are likely due to the much lower transfection efficiency of HeLa cells compared to HEK293T cells. Overall, orthogonality scores in HeLa cells were comparable to those in HEK293T cells.

MOBE1 and MOBE3 (which employ different adenosine deaminase domains) were then selected to evaluate in SH-SY5Y cells at the RNF2and HEK3 single amplicon protospacer combinations. SH-SY5Y cells were transfected with either of the two MOBE systems and the GFP enrichment plasmid. After 72 hours, GFP+/mCherry+cells were collected by FACS, and editing efficiencies were determined by NGS. Lower overall on-target editing efficiencies were again observed in SH-SY5Y cells compared to comparable experiments in HEK293T cells (FIGS. 24C-D), but again orthogonality scores were comparable to those in HEK293T cells. These data demonstrate that MOBE systems can be utilized in additional cell types beyond HEK293T cells.

### REFERENCES

1. Rehm, H. L. et al. ClinGen—the Clinical Genome Resource. N. Engl. J. Med. 372, 2235-2242 (2015).
2. Starita, L. M. et al. Variant Interpretation: Functional Assays to the Rescue. Am. J. Hum. Genet. 101, 315-325 (2017).
3. Shah, N. et al. Identification of Misclassified ClinVar Variants via Disease Population Prevalence. Am. J. Hum. Genet. 102, 609-619 (2018).
4. Findlay, G. M. et al. Accurate classification of BRCA1 variants with saturation genome editing. Nature 562, 217-222 (2018).
5. Cong, L. et al. Multiplex Genome Engineering Using CRISPR/Cas Systems. Science 339, 819-823 (2013).
6. Kweon, J. et al. Fusion guide RNAs for orthogonal gene manipulation with Cas9 and Cpf1. Nat. Commun. 8, 1723 (2017).
7. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
8. Gaudelli, N. M. et al. Programmable base editing of A⋅T to G⋅C in genomic DNA without DNA cleavage. Nature 551, 464-471 (2017).
9. Anzalone, A. V. et al. Search-and-replace genome editing without double-strand breaks or donor DNA. Nature 576, 149-157 (2019).
10. Jiang, F. et al. Structures of a CRISPR-Cas9 R-loop complex primed for DNA cleavage. Science 351, 867-871 (2016).
11. Landrum, M. J. et al. ClinVar: improving access to variant interpretations and supporting evidence. Nucleic Acids Res. 46, D1062-D1067 (2018).
12. Cooper, D. N., Krawczak, M., Polychronakos, C., Tyler-Smith, C. & Kehrer-Sawatzki, H. Where genotype is not predictive of phenotype: towards an understanding of the molecular basis of reduced penetrance in human inherited disease. Hum. Genet. 132, 1077-1130 (2013).
13. Webber, B. R. et al. Highly efficient multiplex human T cell engineering without double-strand breaks using Cas9 base editors. Nat. Commun. 10, 5222 (2019).
14. Nishimasu, H. et al. Engineered CRISPR-Cas9 nuclease with expanded targeting space. Science 361, 1259-1262 (2018).
15. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
16. Liu, Z. et al. Efficient generation of mouse models of human diseases via ABE- and BE-mediated base editing. Nat. Commun. 9, 2338 (2018).
17. Porto, E. M., Komor, A. C., Slaymaker, I. M. & Yeo, G. W. Base editing: advances and therapeutic opportunities. Nat. Rev. Drug Discov. 19, 839-859 (2020).
18. Zalatan, J. G. et al. Engineering complex synthetic transcriptional programs with CRISPR RNA scaffolds. Cell 60, 339-350 (2015).
19. Ma, H. et al. Multiplexed labeling of genomic loci with dCas9 and engineered sgRNAs using CRISPRainbow. Nat. Biotechnol. 34, 528-530 (2016).
20. Wang, S., Su, J.-H., Zhang, F. & Zhuang, X. An RNA-aptamer-based two-color CRISPR labeling system. Sci. Rep. 6, 26857 (2016).
21. Fukunaga, K. & Yokobayashi, Y. Directed evolution of orthogonal RNA-RBP pairs through library-vs-library in vitro selection. Nucleic Acids Res. gkab527 (2021) doi:10.1093/nar/gkab527.
22. Konermann, S. et al. Genome-scale transcriptional activation by an engineered CRISPR-Cas9 complex. Nature 517, 583-588 (2015).
23. Shechner, D. M., Hacisuleyman, E., Younger, S. T. & Rinn, J. L. Multiplexable, locus-specific targeting of long RNAs with CRISPR-Display. Nat. Methods 12, 664-670 (2015).
24. Hess, G. T. et al. Directed evolution using dCas9-targeted somatic hypermutation in mammalian cells. Nat. Methods 13, 1036-1042 (2016).
25. Valegnrd, K. et al. The three-dimensional structures of two complexes between recombinant MS2 capsids and RNA operator fragments reveal sequence-specific protein-RNA interactions. J. Mol. Biol. 270, 724-738 (1997).
26. Tars, K., Fridborg, K., Bundule, M. & Liljas, L. The Three-Dimensional Structure of Bacteriophage PP7 from Pseudomonas aeruginosa at 3.7-A Resolution. Virology 272, 331-337 (2000).
27. Collantes, J. C. et al. Development and Characterization of a Modular CRISPR and RNA Aptamer Mediated Base Editing System. CRISPR J. 4, 58-68 (2021).
28. Li, C. et al. SWISS: multiplexed orthogonal genome editing in plants with a Cas9 nickase and engineered CRISPR RNA scaffolds. Genome Biol. 21, 141 (2020).
29. Richter, M. F. et al. Phage-assisted evolution of an adenine base editor with improved Cas domain compatibility and activity. Nat. Biotechnol. 38, 883-891 (2020).
30. Gaudelli, N. M. et al. Directed evolution of adenine base editors with increased activity and therapeutic application. Nat. Biotechnol. 38, 892-900 (2020).
31. Wang, Y. et al. sgBE: a structure-guided design of sgRNA architecture specifies base editing window and enables simultaneous conversion of cytosine and adenosine. Genome Biol. 21, 222 (2020).
32. Thuronyi, B. W. et al. Continuous evolution of base editors with expanded target compatibility and improved activity. Nat. Biotechnol. 37, 1070-1079 (2019).
33. Jeong, Y. K. et al. Adenine base editor engineering reduces editing of bystander cytosines. Nat. Biotechnol. 39, 1426-1433 (2021).
34. Riordan, J. D. & Nadeau, J. H. From Peas to Disease: Modifier Genes, Network Resilience, and the Genetics of Health. Am. J. Hum. Genet. 101, 177-191 (2017).
35. Rahit, K. M. T. H. & Tarailo-Graovac, M. Genetic Modifiers and Rare Mendelian Disease. Genes 11, 239 (2020).
36. Kleinstiver, B. P. et al. High-fidelity CRISPR-Cas9 nucleases with no detectable genome-wide off-target effects. Nature 529, 490-495 (2016).
37. Bravo, J. P. K. et al. Structural basis for mismatch surveillance by CRISPR-Cas9. Nature 603, 343-347 (2022).
38. Nelson, J. W. et al. Engineered pegRNAs improve prime editing efficiency. Nat. Biotechnol. (2021) doi:10.1038/s41587-021-01039-7.
39. Mukherjee, S. et al. Identifying digenic disease genes using machine learning in the undiagnosed diseases network. http://biorxiv.org/lookup/doi/10.1101/2020.05.31.125716 (2020) doi:10.1101/2020.05.31.125716.
40. Nachtegael, C. et al. Scaling up oligogenic diseases research with OLIDA: the Oligogenic Diseases Database. Database 2022, baac023 (2022).
41. Wang, C. et al. Microbial single-strand annealing proteins enable CRISPR gene-editing tools with improved knock-in efficiencies and reduced off-target effects. Nucleic Acids Res. 49, e36-e36 (2021).
42. Najm, F. J. et al. Orthologous CRISPR-Cas9 enzymes for combinatorial genetic screens. Nat. Biotechnol. 36, 179-189 (2018).
43. McCarty, N. S., Graham, A. E., Studeni, L. & Ledesma-Amaro, R. Multiplexed CRISPR technologies for gene editing and transcriptional regulation. Nat. Commun. 11, 1281 (2020).
44. Vasquez, C. A., Cowan, Q. T. & Komor, A. C. Base Editing in Human Cells to Produce Single-Nucleotide-Variant Clonal Cell Lines. Curr. Protoc. Mol. Biol. 133, (2020).
45. Yu, Y. et al. Cytosine base editors with minimized unguided DNA and RNA off-target events and high on-target activity. Nat. Commun. 11, 2052 (2020).
46. Adikusuma, F., Pfitzner, C. & Thomas, P. Q. Versatile single-step-assembly CRISPR/Cas9 vectors for dual gRNA expression. PLOS ONE 12, e0187236 (2017).
47. Vakulskas, C. A. et al. A high-fidelity Cas9 mutant delivered as a ribonucleoprotein complex enables efficient gene editing in human hematopoietic stem and progenitor cells. Nat. Med. 24, 1216-1224 (2018).
48. Doman, J. L., Raguram, A., Newby, G. A. & Liu, D. R. Evaluation and minimization of Cas9-independent off-target DNA editing by cytosine base editors. Nat. Biotechnol. 38, 620-628 (2020).
49. Tsai, S. Q. et al. GUIDE-seq enables genome-wide profiling of off-target cleavage by CRISPR-Cas nucleases. Nat. Biotechnol. 33, 187-197 (2015).
50. Doench, J. G. et al. Optimized sgRNA design to maximize activity and minimize off-target effects of CRISPR-Cas9. Nat. Biotechnol. 34, 184-191 (2016).
51. Rees, H. A., Wilson, C., Doman, J. L. & Liu, D. R. Analysis and minimization of cellular RNA editing by DNA adenine base editors. Sci. Adv. 5, eaax5717 (2019).
52. Adikusuma, F., Pfitzner, C. & Thomas, P. Q. Versatile single-step-assembly CRISPR/Cas9 vectors for dual gRNA expression. PLOS ONE 12, e0187236 (2017).
53. Clement, K. et al. CRISPResso2 provides accurate and rapid genome editing sequence analysis. Nat. Biotechnol. 37, 224-226 (2019).
54. Li, H. & Durbin, R. Fast and accurate short read alignment with Burrows-Wheeler transform. Bioinformatics 25, 1754-1760 (2009).
55. Li, H. et al. The Sequence Alignment/Map format and SAMtools. Bioinformatics 25, 2078-2079 (2009)

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A multiplexed orthogonal base editor (MOBE) system, comprising one or more aptamer-based base editor (BE) system that comprises an aptamer-gRNA construct, wherein the aptamer-gRNA construct comprises a DNA modifier recruited directly to its gRNA via an aptamer-binding interaction, and wherein the aptamer-gRNA construct is combined with a corresponding coat protein-deaminase fusion to form a gRNA-aptamer-coat protein-deaminase complex.

2. The MOBE system of claim 1, wherein the DNA modifier is cytosine deaminase and/or adenosine deaminase.

3. The MOBE system of claim 1, wherein the aptamer-based BE system is a combination of a Cytidine base editor (CBE) system and Adenine base editor (ABE) system.

4. The MOBE system of claim 3, wherein the ABE system comprises an evolved TadA deaminase.

5. The MOBE system of claim 3, wherein the CBE system comprises evoSPOBRC2 cytosine deaminase.

6. The MOBE system of claim 3, wherein the gRNA is derived from a single CRISPR/Cas9 protein.

7. The MOBE system of claim 3, wherein the CBE system and the ABE system are orthogonal to each other.

8. The MOBE system of claim 1, wherein each MOBE is a (Sp)-nCas9 variant, comprises apt-CBE and apt-ABE, and wherein each MOBE comprises an amino acid sequence selected from the group consisting of SEQ ID NO:20 (MOBE1); SEQ ID NO:21 (MOBE2), SEQ ID NO:22 (MOBE3), and SEQ ID NO:23 (MOBE4).

9. The MOBE system of claim 1, wherein the gRNA-aptamer-coat protein-deaminase complex introduces combinations of only C⋅G to T⋅A or A⋅T to G⋅C point mutations simultaneously at each site of a genomic locus.

10. A reporter plasmid comprising the MOBE system of any one of claim 1, wherein the reporter plasmid facilitates the enrichment of cells with orthogonal multiplexed edits and increased editing efficiency.

11. The reporter plasmid of claim 10, wherein the reporter is a fluorescence-based reporter.

12. The reporter plasmid of claim 11, wherein each report plasmid comprises an amino acid sequence set forth in SEQ ID NO:24.

13. A method of making the MOBE system of claim 1, comprising a) constructing one or more aptamer-gRNA constructs; and b) combining the aptamer-gRNA construct with a corresponding coat protein-deaminase fusion to form a gRNA-aptamer-coat protein-deaminase complex.

14. The method of claim 13, wherein the aptamer-gRNA construct comprises a DNA modifier recruited directly to its gRNA via an aptamer-binding interaction.

15. Use of the MOBE system of claim 1 for therapeutic correction of polygenic disorders, modeling of polygenic disorders, and/or a gene editing for treatment.

16. Use of the MOBE system of claim 1 for implementing additional Sp-nCas9 variants.

17. Use of the MOBE system of claim 1 for base editor screens to probe epistasis/synthetic lethal gene interactions, metabolic reprogramming, genetic logic circuits, and event recording.

18. Use of the reporter plasmid of claim 10 for therapeutic correction of polygenic disorders, modeling of polygenic disorders, and/or a gene editing for treatment.

19. Use of the reporter plasmid of claim 10 for implementing additional Sp-nCas9 variants.

20. Use of the reporter plasmid of claim 10 for base editor screens to probe epistasis/synthetic lethal gene interactions, metabolic reprogramming, genetic logic circuits, and event recording.


SEQUENCE LISTING (28 sequences)

SEQ ID NO: 1 - RNA, 96 nt, synthetic construct
  source 1..96: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttgaaaaagtggcaccgagtcggtgc

SEQ ID NO: 2 - RNA, 121 nt, synthetic construct
  source 1..121: mol_type = other RNA
  misc_difference 1..20: note = a, c, t, g, unknown, or other
nnnnnnnnnnnnnnnnnnnngttttagagctacctaaggagtttatatggaaacccttaggtagcaagttaaaataaggctagtccgttatcaacttgaaaaagtggcaccgagtcggtgc

SEQ ID NO: 3 - RNA, 121 nt, synthetic construct
  source 1..121: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttcctaaggagtttatatggaaacccttaggaagtggcaccgagtcggtgc

SEQ ID NO: 4 - RNA, 127 nt, synthetic construct
  source 1..127: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttgaaaaagtggcaccgagtcggtgcaacataaggagtttatatggaaacccttatg

SEQ ID NO: 5 - RNA, 111 nt, synthetic construct
  source 1..111: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttgggccctgaagaagggcccaagtggcaccgagtcggtgc

SEQ ID NO: 6 - RNA, 117 nt, synthetic construct
  source 1..117: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttgaaaaagtggcaccgagtcggtgcgcgggccctgaagaagggccc

SEQ ID NO: 7 - RNA, 111 nt, synthetic construct
  source 1..111: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttctgaatgcctgcgagcatcaagtggcaccgagtcggtgc

SEQ ID NO: 8 - RNA, 115 nt, synthetic construct
  source 1..115: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttgaaaaagtggcaccgagtcggtgcctgaatgcctgcgagcatc

SEQ ID NO: 9 - RNA, 126 nt, synthetic construct
  source 1..126: mol_type = other RNA
  misc_difference 1..20: note = a, c, t, g, unknown, or other
nnnnnnnnnnnnnnnnnnnngttttagagctaggccaacatgaggatcacccatgtctgcagggcctagcaagttaaaataaggctagtccgttatcaacttgaaaaagtggcaccgagtcggtgc

SEQ ID NO: 10 - RNA, 122 nt, synthetic construct
  source 1..122: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttccaacatgaggatcacccatgtctgcagggaagtggcaccgagtcggtgc

SEQ ID NO: 11 - RNA, 116 nt, synthetic construct
  source 1..116: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttgaaaaagtggcaccggcacatgaggatcacccatgtgccggtgc

SEQ ID NO: 12 - RNA, 121 nt, synthetic construct
  source 1..121: mol_type = other RNA
  misc_difference 1..20: note = a, c, t, g, unknown, or other
nnnnnnnnnnnnnnnnnnnngttttagagctagaaatagcaagttaaaataaggctagtccgttatcaacttgaaaaagtggcaccgagtcggtgcgcgcacatgaggatcacccatgtgc

SEQ ID NO: 13 - protein, 1676 aa, synthetic construct
MKRTADGSEFESPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSKRTADGSEFEPKKKRKVGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGGSPKKKRKV

SEQ ID NO: 14 - protein, 1676 aa, synthetic construct
MKRTADGSEFESPKKKRKVDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKVGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGGSPKKKRKV

SEQ ID NO: 15 - protein, 1676 aa, synthetic construct
MKRTADGSEFESPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSKRTADGSEFEPKKKRKVGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGGSPKKKRKV

SEQ ID NO: 16 - protein, 629 aa, synthetic construct
MKRTADGSEFESPKKKRKVKSIRCKNCNKLLFKADSFDHIEIRCPRCKRHIIMLNACEHPTEKHCGKREKITHSDETVRYGGGGTGGGGSAEYVRALFDFNGNDEEDLPFKKGDILRIRDKPEEQWWNAEDSEGKRGMILVPYVEKYSGDYKDHDGDYKDHDIDYKDDDDKSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGSPKKKRKV

SEQ ID NO: 17 - protein, 298 aa, synthetic construct
MKRTADGSEFESPKKKRKVSSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSKSIRCKNCNKLLFKADSFDHIEIRCPRCKRHIIMLNACEHPTEKHCGKREKITHSDETVRYGSKRTADGSEFEPKKKRKV

SEQ ID NO: 18 - protein, 892 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSSGGSSGGSSGSETPGTSESATPESSGGSSGGSSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLKSGGSSGGSSGSETPGTSESATPESSGGSSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSGATNFSLLKQAGDVEENPGPMVSKGEELFTGVVPILVELDGDVNGHKFSVSGEGEGDATYGKLTPKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGSPKKKRKV

SEQ ID NO: 19 - protein, 854 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSSGGSSGGSSGGSSGGSSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLKSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSGATNFSLLKQAGDVEENPGPMVSKGEELFTGVVPILVELDGDVNGHKFSVSGEGEGDATYGKLTPKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPALLPDNHYLSTQSALSKDPNEKRDHMVLLEFVTAAGITLGMDELYKSGGSPKKKRKV

SEQ ID NO: 20 - protein, 1020 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSSGGSSGGSSGSETPGTSESATPESSGGSSGGSSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLHHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLKSGGSSGGSSGSETPGTSESATPESSGGSSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSGATNFSLLKQAGDVEENPGPGSGEGRGSLLTCGDVEENPGPMKRTADGSEFESPKKKRKVKSIRCKNCNKLLFKADSFDHIEIRCPRCKRHIIMLNACEHPTEKHCGKREKITHSDETVRYGGGGTGGGGSAEYVRALFDFNGNDEEDLPFKKGDILRIRDKPEEQWWNAEDSEGKRGMILVPYVEKYSGDYKDHDGDYKDHDIDYKDDDDKSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSKRTADGSEFEPKKKRKV

SEQ ID NO: 21 - protein, 961 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSSGGSSGGSSGSETPGTSESATPESSGGSSGGSSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLKSGGSSGGSSGSETPGTSESATPESSGGSSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSGATNFSLLKQAGDVEENPGPGSGEGRGSLLTCGDVEENPGPMKRTADGSEFESPKKKRKVSSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSKSIRCKNCNKLLFKADSFDHIEIRCPRCKRHIIMLNACEHPTEKHCGKREKITHSDETVRYGSKRTADGSEFEPKKKRKV

SEQ ID NO: 22 - protein, 982 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSSGGSSGGSSGGSSGGSSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLKSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSGATNFSLLKQAGDVEENPGPGSGEGRGSLLTCGDVEENPGPMKRTADGSEFESPKKKRKVKSIRCKNCNKLLFKADSFDHIEIRCPRCKRHIIMLNACEHPTEKHCGKREKITHSDETVRYGGGGTGGGGSAEYVRALFDFNGNDEEDLPFKKGDILRIRDKPEEQWWNAEDSEGKRGMILVPYVEKYSGDYKDHDGDYKDHDIDYKDDDDKSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSKRTADGSEFEPKKKRKV

SEQ ID NO: 23 - protein, 923 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSSGGSSGGSSGGSSGGSSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLKSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSGATNFSLLKQAGDVEENPGPGSGEGRGSLLTCGDVEENPGPMKRTADGSEFESPKKKRKVSSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSKSIRCKNCNKLLFKADSFDHIEIRCPRCKRHIIMLNACEHPTEKHCGKREKITHSDETVRYGSKRTADGSEFEPKKKRKV

SEQ ID NO: 24 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRVEVKFEGDTLVNRIELKGIDFKEDGNILGHKLEYNYNSHNVYIMADKQKNGIKVNFKIRHNIEDGSVQLADHYQQNTPIGDGPVLLPDNHYSSTQSALSKDPNEKRDHMVLLEFVTAAGITLGMDELYK

SEQ ID NO: 25 - DNA, 20 bp, Homo sapiens
  source 1..20: mol_type = genomic DNA
attactattattatctgaga

SEQ ID NO: 26 - DNA, 20 bp, Homo sapiens
  source 1..20: mol_type = genomic DNA
gtcgtagccagtccgaaccc

SEQ ID NO: 27 - DNA, 20 bp, Homo sapiens
  source 1..20: mol_type = genomic DNA
gatgttccaatcagtacgca

SEQ ID NO: 28 - DNA, 20 bp, Homo sapiens
  source 1..20: mol_type = genomic DNA
tctgcttctccagccctggc